(19) United States
(12) Patent Application Publication
(10) Pub. No.: US 2023/0330140 A1
(43) Pub. Date: Oct. 19, 2023

(54) IMMUNE CELL DELIVERY OF SIALIDASE CANCER CELLS, IMMUNE CELLS AND THE TUMOR MICROENVIRONMENT

(71) Applicant: Ansun Biopharma, Inc., San Diego, CA (US)

(72) Inventor: Nancy CHANG, San Diego, CA (US)

(21) Appl. No.: 17/779,530

(22) PCT Filed: Nov. 24, 2020
(86) PCT No.: PCT/US2020/062106

§ 371 Date: May 24, 2022

Related U.S. Application Data
(60) Provisional application No. 62/940,188, filed on Nov. 25, 2019.

(51) Int. Cl. A61K 35/17; A61K 38/47; A61P 35/00

(52) U.S. Cl. CPC A61K 35/17; A61K 38/47; A61P 35/00

(57) ABSTRACT

The present application provides methods and compositions for treating cancers (such as solid tumors) using engineered immune cells encoding a sialidase and a chimeric immune receptor. In some embodiments, the engineered immune cell is a CAR-T, CAR-NK, CAR-M, or CAR-NKT cell. In some embodiments, the sialidase is an Actinomyces viscosus sialidase or a derivative thereof, such as DAS 181. In some embodiments, the methods and compositions provided herein reduce sialylation of tumor cells and/or immune cells.

### CROSS REFERENCE TO RELATED APPLICATIONS

This application claims priority benefit of U.S. Provisional Application 62/940,188 filed Nov. 25, 2019, the content of which is incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 208712000540SEQLIST.TXT, date recorded: Nov. 24, 2020, size: 233 KB).

### FIELD

The present application relates to methods and compositions for treating a cancer with an engineered immune cell encoding a sialidase and a chimeric immune receptor.

### BACKGROUND

Cancer is the second leading cause of death in the United States. In recent years, great progress has been made in cancer immunotherapy, including immune checkpoint inhibitors, T cells with chimeric antigen receptors, and oncolytic viruses.

Chimeric antigen receptor T, NK, or NKT cells (also known as CAR-T, CAR-NK, or CAR-NKT cells) are T cells, natural killer (NK) cells, or natural killer T (NKT) cells that have been genetically engineered to produce an artificial immune receptor for use in immunotherapy. CAR-T, -NK, or -NKT cells represent an exciting and new approach to treat cancer by using the patient's own immune system, albeit it modified, as well as allogeneic CAR-NK and CAR-NKT cells to attack cancer cells. The first approved treatments use CARs that target the antigen CD19, present in B-cell-derived cancers such as acute lymphoblastic leukemia (ALL) and diffuse large B-cell lymphoma (DLBCL). Tisagenlecleucel (Kymriah®) is approved to treat relapsed/refractory B-cell precursor acute lymphoblastic leukemia (ALL), while axicabtagene ciloleucel (Yescarta®) is approved to treat relapsed/refractory diffuse large B-cell lymphoma (DLBCL). One problem with the present treatments is that cancer cells tend to mutate over time, losing the CD19 antigen that is targeted by the current treatments. Thus, the challenge of a complete cure with the current CAR-T, CAR-NK, or CAR-NKT treatment has not yet been overcome. There are efforts underway to engineer CARs targeting many other blood cancer antigens, including CD30 in refractory Hodgkin's lymphoma; CD33, CD123, and FLT3 in acute myeloid leukemia (AML); and BCMA in multiple myeloma.

Although there has been some success in blood cancers, solid tumors have presented a more difficult target. Identification of effective antigens has been challenging: such antigens must be highly expressed on the majority of cancer cells, but largely absent on normal tissues. CAR-T cells are also not trafficked efficiently into the center of solid tumor masses, and the hostile tumor microenvironment suppresses T cell activity.

Thus, there is a need for novel engineered immune cells that overcome the challenges faced in treating blood cancers that mutate to evade CAR-Ts, solid tumors, and other cancers that have thus far evaded treatment with engineered immune cells that target cancer antigens.

The present invention addresses these problems with novel engineered immune cells.

### BRIEF SUMMARY

Provided herein are compositions comprising an engineered immune cell (e.g., a CAR-T, CAR-NK, CAR-M, or CAR-NKT) with inserted in its genome a nucleic acid molecule encoding one or more sialidases, including recombinant sialidases. Suitable engineered immune cells (e.g., a CAR-T, CAR-NK, CAR-M, or CAR-NKT) can be created by inserting an expression cassette that includes a sequence encoding a sialidase or a portion thereof with sialidase activity into the engineered immune cell.

Also provided are methods for engineered immune cell (e.g., a CAR-T, CAR-NK, CAR-M, or CAR-NKT) delivery of a sialidase to the tumor microenvironment. Within the tumor microenvironment the sialidase can remove terminal sialic acid residues on cancer cells, immune cells and other types of cells, thereby reducing the barrier for entry of immunotherapy reagents and promote cellular immunity against cancer cells. In one embodiment, the sialidase is a recombinant sialidase. In yet another embodiment, the sialidase is a bacterial derived recombinant sialidase. In yet another embodiment, the bacterial derived recombinant sialidase is DAS181.

In some aspects, the present application provides an engineered immune cell comprising a first heterologous nucleotide sequence encoding a sialidase and a second heterologous nucleotide sequence encoding a chimeric antigen receptor. In some embodiments, the sialidase is a human sialidase. In some embodiments, the sialidase is a bacterial sialidase. In some embodiments, the sialidase is a Neu5Ac alpha(2,6)-Gal sialidase or a Neu5Ac alpha(2,3)-Gal sialidase.

In some aspects, the present application provides a composition comprising a first engineered immune cell comprising a first heterologous nucleotide sequence encoding a sialidase, and a second engineered immune cell comprising a second heterologous nucleotide sequence encoding a chimeric immune receptor. In some embodiments, the first engineered immune cell and the second engineered immune cell are of the same type (e.g., T cell). In some embodiments, the first engineered immune cell and the second engineered immune cell are of different types. In some embodiments, the sialidase is a human sialidase. In some embodiments, the sialidase is a bacterial sialidase. In some embodiments, the sialidase is a Neu5Ac alpha(2,6)-Gal sialidase or a Neu5Ac alpha(2,3)-Gal sialidase.

In some embodiments according to any one of the engineered immune cells or compositions described above, the sialidase is selected from the group consisting of: NEU1, NEU2, NEU3, NEU4 and derivatives thereof.

In some embodiments according to any one of the engineered immune cells or compositions described above, the sialidase is any protein having exo-sialidase activity (Enzyme Commission EC 3.2.1.18). In some embodiments according to any one of the engineered immune cells or compositions described above, the sialidase is selected from the group consisting of Clostridium perfringens sialidase, Actinomyces viscosus sialidase, Arthrobacter ureafaciens sialidase, and derivatives thereof. In some embodiments, the sialidase is an Actinomyces viscosus sialidase or a derivative thereof. In some embodiments according to any one of the engineered immune cells or compositions described above, the sialidase comprises an amino acid sequence having at least about 80% (e.g., at least 85%, 90%, 91%, 92%, 93%, 94%, 95%, 96%, 97%, 98%, 99%, or 100%) sequence identity to an amino acid sequence selected from the group consisting of SEQ ID NOs: 1-28, 31, and 53-54. In some embodiments, the sialidase comprises an amino acid sequence having at least about 80% (e.g., at least 85%, 90%, 91%, 92%, 93%, 94%, 95%, 96%, 97%, 98%, 99%, or 100%) sequence identity to the amino acid sequence of SEQ ID NO: 1 or 2. In some embodiments, the sialidase is DAS181. In some embodiments, the sialidase comprises the amino acid sequence of SEQ ID NO: 2. In some embodiments, the sialidase comprises the amino acid sequence of SEQ ID NO: 27. In some embodiments, the sialidase comprises the amino acid sequence of SEQ ID NO: 28. In some embodiments, the sialidase comprises the amino acid sequence of SEQ ID NO: 31.

In some embodiments according to any one of the engineered immune cells or compositions described above, the sialidase is membrane bound on the engineered immune cell.

In some embodiments according to any one of the engineered immune cells or compositions described above, the sialidase is secreted by the engineered immune cell.

In some embodiments according to any one of the engineered immune cells or compositions described above, the sialidase comprises an anchoring domain. In some embodiments, the anchoring domain is located at the carboxy terminus of the sialidase. In some embodiments, the sialidase is a fusion protein comprising from the N-terminus to the C-terminus: a sialidase catalytic domain and an anchoring domain. In some embodiments, the anchoring domain is positively charged at physiologic pH. In some embodiments, the anchoring domain is a glycosaminoglycan (GAG)-binding domain.

In some embodiments according to any one of the engineered immune cells or compositions described above, the first heterologous nucleotide sequence further encodes a secretion sequence operably linked to the sialidase. In some embodiments, the secretion sequence comprises the amino acid sequence of SEQ ID NO: 40.

In some embodiments according to any one of the engineered immune cells or compositions described above, the sialidase comprises a transmembrane domain. In some embodiments, the transmembrane domain is located at the carboxy terminus of the sialidase. In some embodiments, the sialidase is a fusion protein comprising from the N-terminus to the C-terminus: a sialidase catalytic domain, a linker, and a transmembrane domain.

In some embodiments according to any one of the engineered immune cells or compositions described above, the sialidase is capable of cleaving both α-2,3 and α-2,6 sialic acid linkages.

In some embodiments according to any one of the engineered immune cells or compositions described above, the engineered immune cell is a T-cell, a natural killer (NK) cell, a macrophage, or a natural killer T (NKT) cell. In some embodiments, the engineered immune cell is a T cell. In some embodiments, the engineered immune cell is an NK cell.

In some embodiments according to any one of the engineered immune cells or compositions described above, the chimeric immune receptor is selected from the group consisting of a chimeric antigen receptor (CAR), an engineered T cell receptor (TCR), and a T cell receptor fusion protein (TFP). In some embodiments, the chimeric immune receptor is a chimeric antigen receptor (CAR). In some embodiments, the CAR comprises from the N-terminus to the C-terminus: an antigen-binding domain, a transmembrane domain, one or more co-stimulatory domains, and a primary signaling domain.

In some embodiments according to any one of the engineered immune cells or compositions described above, the chimeric immune receptor specifically recognizes a tumor antigen. In some embodiments, the tumor antigen is selected from the group consisting of group consisting of carcinoembryonic antigen, alphafetoprotein, MUC16, survivin, glypican-3, B7 family members, VISTA, MICA/B, LILRB, CD19, BCMA, NY-ESO-1, CD20, CD22, CD24, CD33, CD38, CD200, CEA, EGFRvIII, Integrin beta 1, Integrin beta 4, GD2, HER2, IGF1R, mesothelin, PSMA, ROR1, WT1, NY-ESO-1, and CDH17. In some embodiments, the tumor antigen is selected from the group consisting of VISTA, MICA/B, LILRB, and CDH17. In some embodiments, the tumor antigen is CD-19. In some embodiments, the tumor antigen is LILRB. In some embodiments, the tumor antigen is CDH17.

In some embodiments according to any one of engineered immune cells or compositions described above, the engineered immune cell further comprises a third heterologous nucleotide sequence encoding a heterologous protein, wherein the heterologous protein is a secreted protein that promotes an inflammatory response or inhibits an immunoinhibitory molecule. In some embodiments, the third heterologous nucleotide sequence encodes a heterologous protein that promotes an M2 to M1 switch in a macrophage population. In some embodiments, the third heterologous nucleotide sequence encodes an anti-LILRB antibody.

In some embodiments according to any one of the engineered immune cells or compositions described above, the first heterologous nucleotide sequence and the second heterologous nucleotide sequence are operably linked to the same promoter. In some embodiments, the first heterologous nucleotide sequence and the second heterologous nucleotide sequence are operably linked to different promoters. In some embodiments, the first and/or second promoters can be endogenous promoters. In some embodiments, the first and/or second promoters can be exogenous promoters. In some embodiments, the first and/or second promoters can be viral promoters. In some embodiments, the first and/or second promoters can be synthetic promoters.

In some embodiments according to any one of the engineered immune cells or compositions described above, the first heterologous nucleotide sequence and/or the second heterologous nucleotide sequence are present in a viral vector (e.g., a lentiviral vector).

In another aspect, the present application provides a pharmaceutical composition comprising any one of the engineered immune cells or compositions described above and a pharmaceutically acceptable carrier.

Another aspect of the present application provides a method of treating a cancer in an individual in need thereof comprising administering to the individual an effective amount of any one of the engineered immune cells, compositions, or pharmaceutical compositions described above.

n some embodiments according to any one of the methods described above, the sialidase reduces sialylation of tumor cells.

Another aspect of the present application provides a method of treating a cancer in an individual in need thereof comprising administering to the individual an effective amount of a first engineered immune cell comprising a first heterologous nucleotide sequence encoding a sialidase and an effective amount of a second engineered immune cell comprising a second heterologous nucleotide sequence encoding a chimeric immune receptor.

### BRIEF DESCRIPTION OF THE DRAWINGS

FIGS. 1A-1H show SNA-detected glycans remaining after DAS181 exposure compared to control (PBS). CFG glycan microarray v3.2 was exposed to 0, 0.5, 5, or 50 nM DAS181 (top to bottom panels) and then remaining glycans were detected with SNA lectin. Information for the top 20 glycans detected by SNA in each graph are listed on the right; glycan number, shorthand glycan name/structure, and relative fluorescence units (RFU) are shown. Glycans with an α2,3-linked sialic acid terminus are shaded in gray and indicated with a star (right), and glycans with an α2,6-linked sialic acid terminus are shaded in gray.

FIGS. 2A-2H show MAL2-detected glycans remaining after DAS181 exposure compared to control (PBS). CFG glycan microarray v3.2 was exposed to 0, 0.5, 5, or 50 nM DAS181 (top to bottom panels) and then remaining glycans were detected with MAL2 lectin. Information for the top 20 glycans detected by MAL2 in each graph are listed on the right; glycan number, shorthand glycan name/structure, and relative fluorescence units (RFU) are shown. Glycans with an α2,3-linked sialic acid terminus are shaded in gray and indicated with a star (right), glycans with an α2,6-linked sialic acid terminus are shaded in gray.

FIG. 3 shows results demonstrating removal of sialic acid post transfection with secreted sialidase comprising an anchoring domain constructs. A549-red cells were transfected with expression constructs for secreted DAS181, DAS185, or Neu2, wherein each sialidase was linked to an anchoring domain. After overnight incubation, transfected cells were lifted and re-seeded in 24-well plate. After an additional 72 hrs, non-transfected cells were treated with 100 nM DAS181 for 2 hrs. Cells were fixed and stained with Biotinylated-MAL II for 1 hr followed by FITC-Streptavidin (for α-2,3 SA) or SNA-FITC (for α-2,6 SA) or PNA-FITC (for galactose) for an additional 1 hr before performing flow. S-: secreted sialidase comprising an anchoring domain.

FIG. 4 shows results demonstrating removal of sialic acid post transfection with transmembrane sialidase constructs. A549-red cells were transfected with expression constructs for secreted DAS181, transmembrane DAS181, DAS185, or Neu2. After overnight incubation, transfected cells were lifted and re-seeded in 24-well plate. After an additional 72 hrs, cells were fixed and stained with Biotinylated-MAL II for 1 hr followed by FITC-Streptavidin (for α-2,3 SA) or SNA-FITC (for α-2,6 SA) or PNA-FITC (for galactose) for an additional 1 hr before performing flow. TM-: transmembrane.

FIG. 5A shows an exemplary design of lentiviral vectors expressing CD19-CAR and Sialidases.

FIG. 5B shows a vector map of an exemplary pCDF1-MCS2-EF1α-copGFP Cloning and Expression Lentivector (SBI, CA) used to construct the lentivirus.

FIGS. 6A-6C shows the transduction efficiency of human NK cells by sialidase lentiviral vectors. Human NK cells were transduced with lentiviruses expressing secreted sialidases comprising an anchoring domain (SP-Sial) or transmembrane sialidases (TM-Sial) at a MOI of 15 for 3 days. GFP expression by NK cells were measured by flow cytometry.

FIGS. 7A-7F show enhancement of NK cell-mediated tumor cell killing by sialidase expression. CD19+ Raji tumor cells at 1×10⁴ cells per well were cultured with 2.5×10e4 per well of total NK cells composed of control NK cells, TM-Sial-NK cells, or SP-Sial-NK cells, each mixed with CD19-CAR-NK at 1:1 ratio. Twenty-four hours later, the cells were collected and subjected to flow analysis. FIGS. 7A-7C shows gating of live Raji tumor cells gated. FIGS. 7D-7F shows analysis of PI staining of Raji tumor cells cultured with CD19-CAR-NK+NK, CD19-CAR-NK+TM-Sial-NK, or CD19-CAR-NK+SP-Sial-NK cell mixtures.

FIG. 8 shows the transduction efficiency of human T cells by sialidase lentiviral vectors. CD3 antibody activated human T cells were transduced with lentivirus at a MOI of 5 and cultured for 3 days. GFP expression of human T cells were measured by flow cytometry.

FIG. 9 shows enhancement of T cell-mediated tumor cell killing by sialidase expression. CD19+ Raji tumor cells at 1×10e4 cells per well were cultured with 5×10e4 per well of total T cells composed of control T cells, TM-Sial-T cells, or SP-Sial-T cells, each mixed with CD19-CAR-T at 1:1. T cells at were added at 1×10e4 cells per well to all the wells. Twenty-four hours later, the cells were subjected to flow analysis. FIG. 9A shows gating of live Raji tumor cells. FIG. 9B shows the percentage of live Raji tumor cells in coculture with the T cells (CD19-CAR-T+T, CD19-CAR-T+TM-Sial-T, or CD19-CAR-T+SP-Sial-T cell mixtures).

FIG. 10 shows enhancement of T cell-mediated tumor cell apoptosis by sialidase expression. CD19+ Raji tumor cells were cultured with control T cells, TM-Sial-T cells, or SP-Sial-T cells, all mixed with CD19-CAR-T in a 1:1 ratio. The T cells were added to all the wells and cultured for 24 hours. The cells were stained with Annexin V and subjected to flow analysis. Raji tumor cells were gated for Annexin V analysis. The number shows MFI (mean fluorescence intensity) of Annexin V staining.

FIGS. 11A-11D show results demonstrating that sialidase expression in T cells reduced sialic acids on tumor cells in co-culture. CD19+ Raji tumor cells were cultured with control T cells, TM-Sial-T cells, or SP-Sial-T cells, all mixed with CD19-CAR-T at a 1:1 ratio. FIGS. 11A-11B show cells were stained with MAL for α-2,3 sialic acids. FIGS. 11C-11D show cells stained with SNA for α-2,6 sialic acids. Cells were subjected to flow analysis. Raji tumor cells were gated for sialic acids expression analysis. The numbers in the histograms indicate MFI of lectin staining, the average of which were plotted in bar graphs in FIG. 11B and FIG. 11D

### DETAILED DESCRIPTION

The present application provides compositions and methods for treating a cancer (e.g., solid tumor) comprising engineered immune cells encoding a sialidase. The sialidase expressed by the engineered immune cells (e.g., CAR-T or CAR-NK cells) can reduce the level of sialic acid residues on the surface of tumor cells. Without wishing to be bound by theory, the high level of sialic acid on tumor cells can serve to interfere with the killing of tumor cells by cells of the immune system such as T cells or NK cells. Without being bound by theory, by eliminating sialic acid residues from the surface of cancer cells, the engineered immune cells encoding sialidase may make the tumor micro-environment less hostile to immune cells, such as CAR-Ts, NK cells and macrophages, allowing the better infiltration of the tumor micro-environment by the engineered immune cells (e.g., the CAR-T, CAR-NK, or CAR-M (CAR-macrophage) cells) expressing sialidase.

In some embodiments, the sialidase is an Actinomyces viscosus sialidase or a derivative thereof. In some embodiments, the sialidase is DAS181 or a derivative thereof. Applicants have unexpectedly discovered that with respect to the desialylation of tumor cells, DAS181 has a higher potency than virtually all other sialidases, including naturally occurring ones, and it is broadly active against all sialic acids no matter the structure of the underlying oligosaccharide chains. DAS181 has the ability to remove sialic acid residues from the surface of cancer cells much more efficiently than other sialidases. This is a discovery that was not expected. For example, DAS181 when expressed in cells, either in a secreted form or anchored on the cell surface, showed unexpected potent activity at removal of tumor cell surface sialic acids in comparison to a human sialidase Neu2 constructed in the same format. The Neu2 showed much lower activity in sialic acid removal from tumor cells.

### I. Definitions

Terms are used herein as generally used in the art, unless otherwise defined as follows.

As used herein, “treatment” or “treating” is an approach for obtaining beneficial or desired results including clinical results. For purposes of this application, beneficial or desired clinical results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, preventing or delaying the occurrence or recurrence of the disease, delay or slowing the progression of the disease, ameliorating the disease state, providing a remission (whether partial or total) of the disease, decreasing the dose of one or more other medications required to treat the disease, delaying the progression of the disease, increasing the quality of life, and/or prolonging survival. Also encompassed by “treatment” is a reduction of pathological consequence of the disease. The methods of the present application contemplate any one or more of these aspects of treatment.

The terms “individual,” “subject” and “patient” are used interchangeably herein to describe a mammal, including humans. In some embodiments, the individual is human. In some embodiments, an individual suffers from a respiratory infection. In some embodiments, the individual is in need of treatment.

As is understood in the art, an “effective amount” refers to an amount of a composition sufficient to produce a desired therapeutic outcome (e.g., reducing the severity or duration of, stabilizing the severity of, or eliminating one or more symptoms of respiratory infection). For therapeutic use, beneficial or desired results include, e.g., decreasing one or more symptoms resulting from the disease (biochemical, histologic and/or behavioral), including its complications and intermediate pathological phenotypes presented during development of the disease, increasing the quality of life of those suffering from the disease, decreasing the dose of other medications required to treat the disease, enhancing effect of another medication, delaying the progression of the disease, and/or prolonging survival of patients. In some embodiments, an effective amount of the therapeutic agent may extend survival (including overall survival and progression free survival); result in an objective response (including a complete response or a partial response); relieve to some extent one or more signs or symptoms of the disease or condition; and/or improve the quality of life of the subject.

As used herein the term “wild type” is a term of the art understood by skilled persons and means the typical form of an organism, strain, gene or characteristic as it occurs in nature as distinguished from mutant or variant forms.

The terms “non-naturally occurring” or “engineered” indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified.

As used herein, “sialidase” refers to a naturally occurring or engineered sialidase that is capable of catalyzing the cleavage of terminal sialic acids from carbohydrates on glycoproteins or glycolipids. In some embodiments according to any one of the engineered immune cells or compositions described above, the sialidase is any protein having exo-sialidase activity (Enzyme Commission EC 3.2.1.18). As used herein, the term “sialidase” encompasses a sialidase catalytic domain protein. A “sialidase catalytic domain protein” is a protein that comprises the catalytic domain of a sialidase, or an amino acid sequence that is substantially homologous to the catalytic domain of a sialidase, but does not comprise the entire amino acid sequence of the sialidase the catalytic domain is derived from, wherein the sialidase catalytic domain protein retains substantially the same activity as the intact sialidase the catalytic domain is derived from. A sialidase catalytic domain protein can comprise amino acid sequences that are not derived from a sialidase, but this is not required. A sialidase catalytic domain protein can comprise amino acid sequences that are derived from or substantially homologous to amino acid sequences of one or more other known proteins, or can comprise one or more amino acid residues that are not derived from or substantially homologous to amino acid sequences of other known proteins.

As used herein, “membrane-associated” describes a protein (e.g., a sialidase) that interacts with an entity that is at or on the exterior surface of a cellor is in close proximity to the exterior surface of a cell, e.g., via an “extracellular anchoring domain” or “anchoring domain.”

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The term “antibody” is used in its broadest sense and encompasses various antibody structures, including but not limited to monoclonal antibodies, polyclonal antibodies, multispecific antibodies (e.g., bispecific antibodies), humanized antibodies, chimeric antibodies, full-length antibodies and antigen-binding fragments thereof, so long as they exhibit the desired antigen-binding activity. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains.

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004, each of which are incorporated herein by reference in their entirety for all purposes).

The term “epitope” as used herein refers to the specific group of atoms or amino acids on an antigen to which an antibody binds. Two antibodies or antibody moieties may bind the same epitope within an antigen if they exhibit competitive binding for the antigen.

The terms “polypeptide” or “peptide” are used herein to encompass all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

As use herein, the terms “specifically binds,” “specifically recognizing,” and “is specific for” refer to measurable and reproducible interactions, such as binding between a target and an antibody. In certain embodiments, specific binding is determinative of the presence of the target in the presence of a heterogeneous population of molecules, including biological molecules (e.g., tumor antigen). For example, an antibody that specifically recognizes a target (which can be an epitope) is an antibody that binds this target with greater affinity, avidity, more readily, and/or with greater duration than its bindings to other molecules. In some embodiments, the extent of binding of an antibody to an unrelated molecule is less than about 10% of the binding of the antibody to the target as measured, e.g., by a radioimmunoassay (RIA). In some embodiments, an antibody that specifically binds a target has a dissociation constant (KD) of ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M. In some embodiments, an antibody specifically binds an epitope on a protein that is conserved among the protein from different species. In some embodiments, specific binding can include, but does not require exclusive binding. Binding specificity of the antibody or antigen-binding domain can be determined experimentally by methods known in the art. Such methods comprise, but are not limited to Western blots, ELISA, RIA, ECL, IRMA, EIA, BIACORE™ and peptide scans.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to one or more ingredients in a pharmaceutical formulation, other than an active ingredient, which is nontoxic to a subject. A pharmaceutically acceptable carrier includes, but is not limited to, a buffer, excipient, stabilizer, cryoprotectant, tonicity agent, preservative, and combinations thereof. Pharmaceutically acceptable carriers or excipients have preferably met the required standards of toxicological and manufacturing testing and/or are included on the Inactive Ingredient Guide prepared by the U.S. Food and Drug administration or other state/federal government or listed in the U.S. Pharmacopeia or other generally recognized pharmacopeia for use in mammals, and more particularly in humans.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a disease or condition (e.g., respiratory infection), or a probe for specifically detecting a biomarker described herein. In certain embodiments, the manufacture or kit is promoted, distributed, or sold as a unit for performing the methods described herein.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat disease of type X means the method is used to treat disease of types other than X.

The term “about X-Y” used herein has the same meaning as “about X to about Y.”

As used herein and in the appended claims, the singular forms “a,” “an,” or “the” include plural referents unless the context clearly dictates otherwise.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Compositions

The present application provides compositions comprising engineered immune cells, which can be used for treating a cancer in an individual in need thereof.

In some embodiments, the present application provides an engineered immune cell comprising a first heterologous nucleotide sequence encoding a sialidase and a second heterologous nucleotide sequence encoding a chimeric immune receptor. In some embodiments, the engineered immune cell is a T-cell, a natural killer (NK) cell, or a natural killer T (NKT) cell. In some embodiments, the engineered immune cell is a T-cell. In some embodiments, the engineered immune cell is a NK cell. In some embodiments, the first heterologous nucleotide sequence and the second heterologous nucleotide sequence are operably linked to the same promoter. In some embodiments, the first heterologous nucleotide sequence and the second heterologous nucleotide sequence are operably linked to different promoters. In some embodiments, first heterologous nucleotide sequence and/or the second heterologous nucleotide sequence are present in a lentiviral vector.

In some embodiments, there is provided an engineered immune cell comprising a heterologous nucleotide sequence encoding a bacterial sialidase. In some embodiments, the engineered immune cell is a T cell or NK cell. In some embodiments, the engineered immune cell encodes a CAR. In some embodiments, the engineered immune cell is a CAR-T cell. In some embodiments, the engineered immune cell is a CAR-NK cell. In some embodiments, the sialidase comprises an anchoring domain, e.g., the sialidase is a fusion protein comprising from the N-terminus to the C-terminus: a bacterial sialidase catalytic domain and an anchoring domain. In some embodiments, the anchoring domain is positively charged at physiologic pH, e.g., a glycosaminoglycan (GAG)-binding domain. In some embodiments, the first heterologous nucleotide sequence further encodes a secretion sequence operably linked to the sialidase. In some embodiments, the sialidase comprises a transmembrane domain, e.g., the sialidase is a fusion protein comprising from the N-terminus to the C-terminus: a bacterial sialidase catalytic domain and a transmembrane domain.

In some embodiments, there is provided an engineered immune cell comprising a heterologous nucleotide sequence encoding an Actinomyces viscosus sialidase. In some embodiments, the engineered immune cell is a T cell or NK cell. In some embodiments, the engineered immune cell encodes a CAR. In some embodiments, the engineered immune cell is a CAR-T cell. In some embodiments, the engineered immune cell is a CAR-NK cell. In some embodiments, the sialidase comprises an anchoring domain, e.g., the sialidase is a fusion protein comprising from the N-terminus to the C-terminus: an Actinomyces viscosus sialidase catalytic domain and an anchoring domain. In some embodiments, the anchoring domain is positively charged at physiologic pH, e.g., a glycosaminoglycan (GAG)-binding domain. In some embodiments, the first heterologous nucleotide sequence further encodes a secretion sequence operably linked to the sialidase. In some embodiments, the sialidase comprises a transmembrane domain, e.g., the sialidase is a fusion protein comprising from the N-terminus to the C-terminus: an Actinomyces viscosus sialidase catalytic domain and a transmembrane domain.

In some embodiments, there is provided an engineered immune cell comprising a heterologous nucleotide sequence encoding a secretion sequence (e.g., a eukaryotic signal peptide) operably linked to a sialidase. In some embodiments, the sialidase is secreted from the engineered immune cell. In some embodiments, the sialidase is membrane-associated, e.g., via an anchoring domain. In some embodiments, the sialidase is a fusion protein comprising from the N-terminus to the C-terminus: a sialidase catalytic domain and an anchoring domain. In some embodiments, the anchoring domain is positively charged at physiologic pH, e.g., a glycosaminoglycan (GAG)-binding domain. In some embodiments, the sialidase comprises an amino acid sequence having at least about 80% (e.g., at least 85%, 90%, 91%, 92%, 93%, 94%, 95%, 96%, 97%, 98%, 99%, or 100%) sequence identity to the amino acid sequence of SEQ ID NO: 28. In some embodiments, the sialidase comprises the amino acid sequence of SEQ ID NO: 28. In some embodiments, the engineered immune cell is a T cell or NK cell. In some embodiments, the engineered immune cell encodes a CAR. In some embodiments, the engineered immune cell is a CAR-T cell. In some embodiments, the engineered immune cell is a CAR-NK cell.

In some embodiments, there is provided an engineered immune cell comprising a heterologous nucleotide sequence encoding a sialidase operably linked to a transmembrane domain. In some embodiments, the sialidase is a fusion protein comprising from the N-terminus to the C-terminus: a sialidase catalytic domain and a transmembrane domain. In some embodiments, the sialidase comprises a linker (e.g., a hinge domain of an immunoglobulin) connecting the sialidase catalytic domain to the transmembrane domain. In some embodiments, the sialidase comprises an amino acid sequence having at least about 80% (e.g., at least 85%, 90%, 91%, 92%, 93%, 94%, 95%, 96%, 97%, 98%, 99%, or 100%) sequence identity to the amino acid sequence of SEQ ID NO: 31. In some embodiments, the sialidase comprises the amino acid sequence of SEQ ID NO: 31. In some embodiments, the engineered immune cell is a T cell or NK cell. In some embodiments, the engineered immune cell encodes a CAR In some embodiments, the engineered immune cell is a CAR-T cell. In some embodiments, the engineered immune cell is a CAR-NK cell.

In some embodiments, there is provided an engineered immune cell comprising a heterologous nucleotide sequence encoding DAS181. In some embodiments, the engineered immune cell is a T cell or NK cell. In some embodiments, the engineered immune cell encodes a CAR In some embodiments, the engineered immune cell is a CAR-T cell. In some embodiments, the engineered immune cell is a CAR-NK cell. In some embodiments, the first heterologous nucleotide sequence further encodes a secretion sequence operably linked to the sialidase. In some embodiments, the sialidase comprises a transmembrane domain, e.g., the sialidase is a fusion protein comprising from the N-terminus to the C-terminus: a DAS181 sialidase catalytic domain (i.e., DAS181 without an anchoring domain) and a transmembrane domain.

In some embodiments, there is provided an engineered immune cell comprising a heterologous nucleotide sequence encoding a CAR, wherein the CAR specifically recognizes a tumor antigen selected from: carcinoembryonic antigen, alphafetoprotein, MUC16, survivin, glypican-3, B7 family members, LILRB, CD19, BCMA, NY-ESO-1, CD20, CD22, CD33, CD38, CEA, EGFR (such as EGFRvIII), GD2, HER2, IGF1R, mesothelin, PSMA, ROR1, WT1, NY-ESO-1, CDH17, and other tumor antigens with clinical significance. In some embodiments, the engineered immune cell further comprises a heterologous nucleotide sequence encoding a sialidase. In some embodiments, the engineered immune cell is a T cell or NK cell. In some embodiments, the sialidase is a secreted membrane-associated form of a sialidase (e.g., a sialidase comprising an anchoring domain). In some embodiments, the sialidase is a fusion protein comprising from the N-terminus to the C-terminus: a sialidase catalytic domain and an anchoring domain. In some embodiments, the anchoring domain is positively charged at physiologic pH, e.g., a glycosaminoglycan (GAG)-binding domain. In some embodiments, the first heterologous nucleotide sequence further encodes a secretion sequence operably linked to the sialidase. In some embodiments, the sialidase comprises a transmembrane domain, e.g., the sialidase is a fusion protein comprising from the N-terminus to the C-terminus: a sialidase catalytic domain and a transmembrane domain.

Suitable sialidases are described in the “Sialidase” subsection below. In some embodiments, the sialidase is a Neu5Ac alpha(2,6)-Gal sialidase or a Neu5Ac alpha(2,3)-Gal sialidase. In some embodiments, the sialidase is a human sialidase. In some embodiments, the sialidase is a human sialidase (e.g., NEU2, NEU4) or a derivative thereof.

In some embodiments, the sialidase is a bacterial sialidase (e.g., a Clostridium perfringens sialidase, Actinomyces viscosus sialidase, or Arthrobacter ureafaciens sialidase) or a derivative thereof. In some embodiments, the sialidase is an Actinomyces viscosus sialidase or a derivative thereof the sialidase comprises an amino acid sequence having at least about 80% (e.g., at least 85%, 90%, 91%, 92%, 93%, 94%, 95%, 96%, 97%, 98%, 99%, or 100%) sequence identity to an amino acid sequence selected from the group consisting of SEQ ID NOs: 1-28, 31, and 53-54. In some embodiments, the sialidase comprises an amino acid sequence having at least about 80% (e.g., at least 85%, 90%, 91%, 92%, 93%, 94%, 95%, 96%, 97%, 98%, 99%, or 100%) sequence identity to the amino acid sequence of SEQ ID NO: 1 or 2. In some embodiments, the sialidase is DAS181. In some embodiments, the sialidase comprises the amino acid sequence of SEQ ID NO: 2. In some embodiments, the sialidase comprises the amino acid sequence of SEQ ID NO: 27. In some embodiments, the sialidase comprises the amino acid sequence of SEQ ID NO: 28. In some embodiments, the sialidase comprises the amino acid sequence of SEQ ID NO: 31.

In some embodiments, the sialidase is membrane bound on the engineered immune cell. In some embodiments, the sialidase is secreted by the engineered immune cell.

In some embodiments, the sialidase comprises an anchoring domain. In some embodiments, the sialidase is a fusion protein comprising from the N-terminus to the C-terminus: a sialidase catalytic domain and an anchoring domain. In some embodiments, the anchoring domain is positively charged at physiologic pH. In some embodiments, the anchoring domain is a glycosaminoglycan (GAG)-binding domain. In some embodiments, the first heterologous nucleotide sequence further encodes a secretion sequence operably linked to the sialidase. In some embodiments, the secretion sequence comprises the amino acid sequence of SEQ ID NO: 40.

In some embodiments, the sialidase comprises a transmembrane domain. In some embodiments, the sialidase is a fusion protein comprising from the N-terminus to the C-terminus: a sialidase catalytic domain, a hinge region, and a transmembrane domain. In some embodiments, the anchoring domain or the transmembrane moiety is located at the carboxy terminus of the sialidase. In some embodiments, the sialidase is capable of cleaving both α-2,3 and α-2,6 sialic acid linkages.

Suitable engineered immune cells are described in the “Engineered immune cells” subsection below. In some embodiments, the engineered immune cell is a T cell, NK cell, NKT cell, or macrophage. In some embodiments, the engineered immune cell encodes or expresses an engineered immune receptor. Any engineered immune receptors known in the art may be used, including, for example, the engineered immune receptors described in the “Engineered immune cells” subsection below. In some embodiments, the chimeric immune receptor is selected from the group consisting of a chimeric antigen receptor (CAR), an engineered T cell receptor (TCR), and a T cell receptor fusion protein (TFP). In some embodiments, the chimeric immune receptor is a chimeric antigen receptor (CAR). In some embodiments, the CAR comprises from the N-terminus to the C-terminus: an antigen-binding domain, a transmembrane domain, a co-stimulatory domain, and a primary signaling domain.

In some embodiments, the chimeric immune receptor specifically recognizes a tumor antigen. In some embodiments, the tumor antigen is selected from the group consisting of EGFRvIII, PD-L1, EpCAM, carcinoembryonic antigen, alphafetoprotein, MUC16, survivin, glypican-3, B7 family members, CDH17, LILRB, and CD-19. In some embodiments, the tumor antigen is CD-19. In some embodiments, the chimeric immune receptor specifically recognizes one or more tumor antigens selected from the group consisting of carcinoembryonic antigen, alphafetoprotein, MUC16, survivin, glypican-3, B7 family members, LILRB, CD19, BCMA, NY-ESO-1, CD20, CD22, CD33, CD38, CEA, EGFR (such as EGFRvIII), GD2, HER2, IGF1R, mesothelin, PSMA, ROR1, WT1, NY-ESO-1, CDH17, and other tumor antigens with clinical significance. In some embodiments, the chimeric immune receptor specifically recognizes LILRB.

In some embodiments, there is provided a composition comprising an engineered immune cell that specifically recognizes an tumor-associated or tumor-specific antigen. In some embodiments, the engineered immune cell expresses a sialidase (e.g., an Actinomyces viscosus sialidase or a derivative thereof, such as DAS181). Tumor-associated antigens can include but are not limited to carcinoembryonic antigen, alphafetoprotein, MUC16, survivin, glypican-3, B7 family members, LILRB, CD19, BCMA, NY-ESO-1, CD20, CD22, CD33, CD38, CEA, EGFR (such as EGFRvIII), GD2, HER2, IGF1R, mesothelin, PSMA, ROR1, WT1, NY-ESO-1, CDH17, and other tumor antigens with clinical significance.

In some embodiments, there is provided an engineered T cell comprising a first heterologous nucleotide sequence encoding a sialidase and a second heterologous nucleotide sequence encoding a chimeric immune receptor (e.g., a CAR, a TCR, or a TFP). In some embodiments, the sialidase is a human sialidase. In some embodiments, the sialidase is a bacterial sialidase (e.g., an Actinomyces viscosus sialidase or a derivative thereof, such as DAS181 or a derivative thereof). In some embodiments, the sialidase comprises an anchoring domain, e.g., the sialidase is a fusion protein comprising from the N-terminus to the C-terminus: a sialidase catalytic domain and an anchoring domain. In some embodiments, the anchoring domain is positively charged at physiologic pH, e.g., a glycosaminoglycan (GAG)-binding domain. In some embodiments, the first heterologous nucleotide sequence further encodes a secretion sequence operably linked to the sialidase. In some embodiments, the sialidase comprises a transmembrane domain. In some embodiments, the chimeric immune receptor is a CAR. In some embodiments, the T cell is an anti-CD19 CAR-T cell. In some embodiments, the chimeric immune receptor specifically recognizes one or more tumor antigens such as carcinoembryonic antigen, alphafetoprotein, MUC16, survivin, glypican-3, B7 family members, LILRB, CD19, BCMA, NY-ESO-1, CD20, CD22, CD33, CD38, CEA, EGFR (such as EGFRvIII), GD2, HER2, IGF1R, mesothelin, PSMA, ROR1, WT1, NY-ESO-1, CDH17, and other tumor antigens with clinical significance.

In some embodiments, there is provided an engineered NK cell comprising a first heterologous nucleotide sequence encoding a sialidase and a second heterologous nucleotide sequence encoding a chimeric immune receptor (e.g., a CAR, a TCR, or a TFP). In some embodiments, the sialidase is a human sialidase. In some embodiments, the sialidase is a bacterial sialidase (e.g., an Actinomyces viscosus sialidase or a derivative thereof, such as DAS181 or a derivative thereof). In some embodiments, the sialidase comprises an anchoring domain, e.g., a fusion protein comprising from the N-terminus to the C-terminus: a sialidase catalytic domain and an anchoring domain. In some embodiments, the anchoring domain is positively charged at physiologic pH, e.g., a glycosaminoglycan (GAG)-binding domain. In some embodiments, the first heterologous nucleotide sequence further encodes a secretion sequence operably linked to the sialidase. In some embodiments, the sialidase comprises a transmembrane domain. In some embodiments, the chimeric immune receptor is a CAR. In some embodiments, T cell is an anti-CD19 CAR-NK cell. In some embodiments, the chimeric immune receptor specifically recognizes one or more tumor antigens such as carcinoembryonic antigen, alphafetoprotein, MUC16, survivin, glypican-3, B7 family members, LILRB, CD19, BCMA, NY-ESO-1, CD20, CD22, CD33, CD38, CEA, EGFR (such as EGFRvIII), GD2, HER2, IGF1R, mesothelin, PSMA, ROR1, WT1, NY-ESO-1, CDH17, and other tumor antigens with clinical significance.

In some embodiments, the present application provides a composition comprising a first engineered immune cell comprising a first heterologous nucleotide sequence encoding a sialidase, and a second engineered immune cell comprising a second heterologous nucleotide sequence encoding a chimeric immune receptor. In some embodiments, the sialidase is a human sialidase. In some embodiments, the sialidase is a bacterial sialidase (e.g., an Actinomyces viscosus sialidase or a derivative thereof, such as DAS181 or a derivative thereof). In some embodiments, the sialidase comprises an anchoring domain, e.g., the sialidase is a fusion protein comprising from the N-terminus to the C-terminus: a sialidase catalytic domain and an anchoring domain. In some embodiments, the anchoring domain is positively charged at physiologic pH, e.g., a glycosaminoglycan (GAG)-binding domain. In some embodiments, the first heterologous nucleotide sequence further encodes a secretion sequence operably linked to the sialidase. In some embodiments, the sialidase comprises a transmembrane domain. In some embodiments, the chimeric immune receptor is a CAR. In some embodiments, the T cell is an anti-CD19 CAR-T cell. carcinoembryonic antigen, alphafetoprotein, MUC16, survivin, glypican-3, B7 family members, LILRB, CD19, BCMA, NY-ESO-1, CD20, CD22, CD33, CD38, CEA, EGFR (such as EGFRvIII), GD2, HER2, IGF1R, mesothelin, PSMA, ROR1, WT1, NY-ESO-1, CDH17, and other tumor antigens with clinical significance. In some embodiments, the first engineered immune cell is a T-cell, a natural killer (NK) cell, or a natural killer T (NKT) cell and the second engineered immune cell is a T-cell, a natural killer (NK) cell, or a natural killer T (NKT) cell. In some embodiments, the second engineered immune cell does not comprise a first heterologous nucleotide sequence encoding a sialidase. In some embodiments, the first and the second engineered immune cells are the same type of cell. In some embodiments, the first and second engineered immune cells are T cells. In some embodiments, the first and second engineered immune cells are NK cells. In some embodiments, the first and the second engineered immune cell are different types of cells. Suitable engineered immune cells are described in the “Engineered immune cells” subsection below. In some embodiments, first heterologous nucleotide sequence and/or the second heterologous nucleotide sequence are each present in a lentiviral vector. In some embodiments, first engineered immune cell and the second engineered immune cell are present in the composition in a 1:5, 1:4, 1:3, 1:2, 1.5:1, 1:1, 1:1.5, 2:1, 3:1, 4:1, or 5:1 ratio. In some embodiments, the first engineered immune cell and the second engineered immune cell are present in the composition in a 1:1 ratio.

In some embodiments, there is provided a composition comprising a first T cell comprising a first heterologous nucleotide sequence encoding a sialidase (e.g., an Actinomyces viscosus sialidase or a derivative thereof, such as DAS181), and a second T cell comprising a second heterologous nucleotide sequence encoding a chimeric immune receptor (e.g., a CAR, a TCR, or a TFP). In some embodiments, the sialidase is a human sialidase. In some embodiments, the sialidase is a bacterial sialidase (e.g., an Actinomyces viscosus sialidase or a derivative thereof, such as DAS181 or a derivative thereof). In some embodiments, the sialidase comprises an anchoring domain, e.g., a fusion protein comprising from the N-terminus to the C-terminus: a sialidase catalytic domain and an anchoring domain. In some embodiments, the anchoring domain is positively charged at physiologic pH, e.g., a glycosaminoglycan (GAG)-binding domain. In some embodiments, the first heterologous nucleotide sequence further encodes a secretion sequence operably linked to the sialidase. In some embodiments, the sialidase comprises a transmembrane domain. In some embodiments, the chimeric immune receptor is a CAR. In some embodiments, the CAR specifically recognizes a tumor antigen (e.g. EGFRvIII, PD-L1, EpCAM, carcinoembryonic antigen, alphafetoprotein, MUC16, survivin, glypican-3, B7 family members, LILRB, or CD-19). In some embodiments, the tumor antigen is CD-19. In some embodiments, the chimeric immune receptor specifically recognizes one or more tumor antigens such as carcinoembryonic antigen, alphafetoprotein, MUC16, survivin, glypican-3, B7 family members, LILRB, CD19, BCMA, NY-ESO-1, CD20, CD22, CD33, CD38, CEA, EGFR (such as EGFRvIII), GD2, HER2, IGF1R, mesothelin, PSMA, ROR1, WT1, NY-ESO-1, CDH17, and other tumor antigens with clinical significance.

In some embodiments, there is provided a composition comprising a first NK cell comprising a first heterologous nucleotide sequence encoding a sialidase (e.g., an Actinomyces viscosus sialidase or a derivative thereof, such as DAS181), and a second NK cell comprising a second heterologous nucleotide sequence encoding a chimeric immune receptor (e.g., a CAR, a TCR, or a TFP). In some embodiments, the sialidase is a human sialidase. In some embodiments, the sialidase is a bacterial sialidase (e.g., an Actinomyces viscosus sialidase or a derivative thereof, such as DAS181 or a derivative thereof). In some embodiments, the sialidase comprises an anchoring domain, e.g., a fusion protein comprising from the N-terminus to the C-terminus: a sialidase catalytic domain and an anchoring domain. In some embodiments, the anchoring domain is positively charged at physiologic pH, e.g., a glycosaminoglycan (GAG)-binding domain. In some embodiments, the first heterologous nucleotide sequence further encodes a secretion sequence operably linked to the sialidase. In some embodiments, the sialidase comprises a transmembrane domain. In some embodiments, the chimeric immune receptor is a CAR. In some embodiments, the CAR specifically recognizes a tumor antigen (e.g. EGFRvIII, PD-L1, EpCAM, carcinoembryonic antigen, alphafetoprotein, MUC16, survivin, glypican-3, B7 family members, LILRB, or CD-19. In some embodiments, the tumor antigen is CD-19. In some embodiments, the tumor antigen is LILRB. In some embodiments, the tumor antigen is CDH17.

In some embodiments, there is provided an engineered immune cell (e.g., a T cell, NK cell, or NKT cell) comprising a heterologous nucleotide sequence encoding a sialidase comprising a transmembrane domain. In some embodiments, the transmembrane domain comprises an amino acid sequence selected from SEQ ID NOs: 45-52. In some embodiments, the engineered immune cell further comprises a second heterologous nucleotide sequence encoding chimeric immune receptor. In some embodiments, the sialidase is derived from an Actinomyces viscosus sialidase. In some embodiments, the heterologous nucleotide sequence encoding the sialidase further encodes a secretion sequence operably linked to the sialidase.

In some embodiments, there is provided an engineered immune cell (e.g., a T cell, NK cell, or NKT cell) comprising a heterologous nucleotide sequence encoding a sialidase comprising an anchoring domain. In some embodiments, the anchoring domain is positively charged at physiologic pH, e.g., a glycosaminoglycan (GAG)-binding domain. In some embodiments, the sialidase is a fusion protein comprising from the N-terminus to the C-terminus: a sialidase catalytic domain and an anchoring domain. In some embodiments, the sialidase is a human sialidase. In some embodiments, the sialidase is a bacterial sialidase (e.g., an Actinomyces viscosus sialidase or a derivative thereof, such as DAS181 or a derivative thereof). In some embodiments, the engineered immune cell further comprises a second heterologous nucleotide sequence encoding chimeric immune receptor. In some embodiments, the chimeric immune receptor is a CAR. In some embodiments, the T cell is an anti-CD19 CAR-T cell. In some embodiments, the chimeric immune receptor specifically recognizes one or more tumor antigens such as carcinoembryonic antigen, alphafetoprotein, MUC16, survivin, glypican-3, B7 family members, LILRB, CD19, BCMA, NY-ESO-1, CD20, CD22, CD33, CD38, CEA, EGFR (such as EGFRvIII), GD2, HER2, IGF1R, mesothelin, PSMA, ROR1, WT1, NY-ESO-1, CDH17, and other tumor antigens with clinical significance. In some embodiments, the heterologous nucleotide sequence encoding the sialidase further encodes a secretion sequence operably linked to the sialidase.

In some embodiments, the first heterologous nucleotide sequence encoding the sialidase is operably linked to a promoter. In some embodiments, the second heterologous nucleotide sequence encoding the chimeric receptor is operably linked to a promoter. In some embodiments, the first and second heterologous nucleotide sequences are operably linked to a first and second promoter, respectively. In some embodiments, the first and second promoter are the same promoter. In some embodiments, the first and the second promoter are different promoters.

In some embodiments, there is provided a first lentiviral vector encoding a sialidase and a second lentiviral vector encoding a chimeric immune receptor. In some embodiments, the first heterologous nucleotide sequence encodes a secretion sequence operably linked to a sialidase, wherein the secretion sequence is capable of mediating secretion of the sialidase from the engineered immune cell. In some embodiments, the first heterologous nucleotide sequence encodes, from 5′ end to 3′ end, a secretion sequence, a sialidase (e.g., a catalytic domain of an Actinomyces viscosus sialidase), and an anchoring domain. In some embodiments, the first heterologous nucleotide sequence encodes a sialidase operably linked to a transmembrane domain. In some embodiments, the first heterologous nucleotide sequence encodes, from 5′end to 3′ end, a secretion sequence, a sialidase (e.g., a catalytic domain of an Actinomyces viscosus sialidase), a hinge domain, and a transmembrane domain. In some embodiments, the second heterologous nucleotide sequence encodes a CAR In some embodiments, the second heterologous nucleotide sequence encodes, from 5′ end to 3′ end, a signal peptide, an antigen-binding domain, a transmembrane domain, a co-stimulatory domain, and a primary signaling domain. In some embodiments, the second heterologous nucleotide sequence encodes, from 5′ end to 3′ end, a signal peptide, an antigen-binding moiety (e.g., an anti-CD19 scFv), a CH2 CH3 transmembrane domain, and a 4-1BB/CD3ζ signaling domain. In some embodiments, the secretion sequence or signal peptide is a CD8 signal peptide. In some embodiments, there is provided a single lentiviral vector comprising the first heterologous nucleotide sequence and the second heterologous nucleotide sequence. In some embodiments, the first and/or second lentiviral vector further comprises a detectable reporter (e.g., a fluorescent reporter protein such as GFP) operably linked to a promoter. In some embodiments, the detectable reporter (e.g., GFP) is operably linked to an EF1-α promoter. Construction of exemplary lentiviral vectors is described in Example 3.

### 1. Sialidase

In some embodiments, the engineered immune cell comprises a heterologous heterologous nucleotide sequence encoding a sialidase that includes all or a catalytic portion of a naturally occurring sialidase that is capable of removing sialic acid (N-acetylneuraminic acid (Neu5Ac)), e.g., from a glycan on a human cell. In some embodiments, the sialidase is any protein having exo-sialidase activity (Enzyme Commission EC 3.2.1.18). In general, Neu5Ac is linked via an alpha 2,3, an alpha 2,6 or alpha 2,8 linkage to the penultimate sugar in glycan on a protein by any of a variety of sialyl transferases. The common human sialyltransferases are summarized in Table 1.

TABLE 1
Nomenclature of Neu5Ac sialyltransferases

| | | | EC | |
|---|---|---|---|---|
| Abbreviation | Resulting Group | Substrate | Number | HGNC |
| ST3Gal I | Neu5Ac-α-(2,3) Gal | Gal-β-1,3-GalNAc | 2.4.99.4 | 10862 |
| ST3Gal II | Neu5Ac-α-(2,3) Gal | Gal-β-1,3-GalNAc | 2.4.99.4 | 10863 |
| ST3Gal III | Neu5Ac-α-(2,3) Gal | Gal-β-1,3(4)-GlcNAc | 2.4.99.6 | 10866 |
| ST3Gal IV | Neu5Ac-α-(2,3) Gal | Gal-β-1,4-GlcNAc | 2.4.99.9 | 10864 |
| ST3Gal V | Neu5Ac-α-(2,3) Gal | Gal-β-1,4-Glc | 2.4.99.9 | 10872 |
| ST3Gal VI | Neu5Ac-α-(2,3) Gal | Gal-β-1,4-GlcNAc | 2.4.99.9 | 18080 |
| ST6Gal I | Neu5Ac-α-(2,6) Gal | Gal-β-1,4-GlcNAc | 2.4.99.1 | 10860 |
| ST6Gal II | Neu5Ac-α-(2,6) Gal | Gal-β-1,4-GlcNAc | 2.4.99.2 | 10861 |
| ST6GalNAc I | Neu5Ac-α-(2,6) GalNAc | GalNAc-α-1,O-Ser/Thr | 2.4.99.7 | 23614 |
| ST6GalNAc II | Neu5Ac-α-(2,6) GalNAc | Gal-β-1,3-GalNAc-α-1,O-Ser/Thr | 2.4.99.7 | 10867 |
| ST6GalNAc III | Neu5Ac-α-(2,6) GalNAc | Neu5Ac-α-2,3-Gal-β-1,3-GalNAc | 2.4.99.7 | 19343 |
| ST6GalNAc IV | Neu5Ac-α-(2,6) GalNAc | Neu5Ac-α-2,3Gal-β-1,3-GalNAc | 2.4.99.7 | 17846 |
| ST6GalNAc V | Neu5Ac-α-(2,6) GalNAc | Neu5Ac-α-2,6-GalNAc-β-1,3-GalNAc | 2.4.99.7 | 19342 |
| ST6GalNAc VI | Neu5Ac-α-(2,6) GalNAc | All a-series gangliosides | 2.4.99.7 | 23364 |
| ST8Sia I | Neu5Ac-α-(2,8)-Neu5Ac | Neu5Ac-α-2,3-Gal-β-1,4-Glc-β-1,1Cer | 2.4.99.8 | 10869 |
| | | (GM3) | | |
| ST8Sia II | Neu5Ac-α-(2,8)-Neu5Ac | Neu5Ac-α-2,3-Gal-β-1,4-GlcNAc | 2.4.99.8 | 10870 |
| ST8Sia III | Neu5Ac-α-(2,8)-Neu5Ac | Neu5Ac-α-2,3-Gal-β-1,4-GlcNAc | 2.4.99.8 | 14269 |
| ST8Sia IV | Neu5Ac-α-(2,8)-Neu5Ac | (Neu5Ac-α-2,8)nNeu5Ac-α-2,3-Gal-β-1-R | 2.4.99.8 | 10871 |
| ST8Sia V | Neu5Ac-α-(2,8)-Neu5Ac | GM1b, GT1b, GD1a, GD3 | 2.4.99.8 | 17827 |
| ST8Sia VI | Neu5Ac-α-(2,8)-Neu5Ac | Neu5Ac-α-2,3(6)-Gal | 2.4.99.8 | 23317 |

HGNC: Hugo Gene Community Nomenclature (world wide web.genenames.org)

The sialidase, in addition to a naturally occurring sialidase or catalytic portion thereof can, optionally, include peptide or protein sequences that contribute to the therapeutic activity of the sialidase. For example, the sialidase protein can include an anchoring domain that promotes interaction between the sialidase and a cell surface. The anchoring domain and sialidase domain can be arranged in any appropriate way that allows the sialidase to bind at or near a target cell membrane such that the therapeutic sialidase can exhibit an extracellular activity that removes sialic acid residues. The sialidase can have more than one anchoring domains. In cases in which the sialidase has more than one anchoring domain, the anchoring domains can be the same or different. The sialidase can comprise one or more transmembrane domains (e.g., one or more transmembrane alpha helices). The sialidase can have more than one sialidase domain. In cases in which a sialidase has more than one sialidase domain, the sialidase domains can be the same or different. Where the sialidase comprises multiple anchoring domains, the anchoring domains can be arranged in tandem (with or without linkers) or on alternate sides of other domains, such as sialidase domains. Where a sialidase comprises multiple sialidase domains, the sialidase domains can be arranged in tandem (with or without linkers) or on alternate sides of other domains.

### Sialidase Catalytic Activity

The sialidase expressed by the engineered immune cell can be specific for Neu5Ac linked via alpha 2,3 linkage, specific for Neu5Ac linked via an alpha 2,6, or can cleave Neu5Ac linked via an alpha 2,3 linkage or an alpha 2,6 linkage. A variety of sialidases are described in Tables 1-5.

A sialidase that can cleave more than one type of linkage between a sialic acid residue and the remainder of a substrate molecule, in particular, a sialidase that can cleave both alpha(2, 6)-Gal and alpha(2, 3)-Gal linkages can be used in the compounds of the disclosure. Sialidases included are the large bacterial sialidases that can degrade the receptor sialic acids Neu5Ac alpha(2,6)-Gal and Neu5Ac alpha(2,3)-Gal. For example, the bacterial sialidase enzymes from Clostridium perfringens (Genbank Accession Number X87369), Actinomyces viscosus (GenBankX62276), Arthrobacter ureafaciens (GenBank Accession Number AY934539), or Micromonospora viridifaciens (Genbank Accession Number D01045) can be used.

In some embodiments, the sialidase comprises all or a portion of the amino acid sequence of a large bacterial sialidase or can comprise amino acid sequences having at least 60%, at least 65%, at least 70%, at least 75%, at least 80%, at least 85%, at least 90%, at least 95%, at least 98%, at least 99%, or 100% sequence identity to all or a portion of the amino acid sequence of a large bacterial sialidase. In some embodiments, the sialidase domain comprises SEQ ID NO: 1, 2 or 27, or a sialidase sequence having at least 60%, at least 65%, at least 70%, at least 75%, at least 80%, at least 85%, at least 90%, at least 95%, at least 98%, at least 99%, or 100% sequence identity to SEQ ID NO: 12. In some embodiments, a sialidase domain comprises the catalytic domain of the Actinomyces viscosus sialidase corresponding to amino acids 274-666 of SEQ ID NO: 26, having at least 60%, at least 65%, at least 70%, at least 75%, at least 80%, at least 85%, at least 90%, at least 95%, at least 98%, at least 99%, or 100% sequence identity to amino acids 274-666 of SEQ ID NO: 26.

Additional sialidases include the human sialidases such as those encoded by the genes NEU2 (SEQ ID NO: 4; Genbank Accession Number Y16535; Monti, E, Preti, Rossi, E., Ballabio, A and Borsani G. (1999) Genomics 57:137-143) and NEU4 (SEQ ID NO: 6; Genbank Accession Number NM080741; Monti et al. (2002) Neurochem Res 27:646-663). Sialidase domains of sialidases of the present disclosure can comprise all or a portion of the amino acid sequences of any sialidase described herein or can comprise amino acid sequences having at least 60%, at least 65%, at least 70%, at least 75%, at least 80%, at least 85%, at least 90%, at least 95%, at least 98%, or at least 99% sequence identity to all or a portion of the amino acid sequences of a sialidase described herein. In some embodiments, where a sialidase domain comprises a portion of the amino acid sequences of a naturally occurring sialidase, or sequences having at least 60%, at least 65%, at least 70%, at least 75%, at least 80%, at least 85%, at least 90%, at least 95%, at least 98%, or at least 99% sequence identity to a portion of the amino acid sequences of a naturally occurring sialidase, the portion comprises essentially the same activity as the naturally occurring sialidase. In some embodiments, the sialidase is a full-length naturally occurring sialidase. In some embodiments, the sialidase expressed by the engineered immune cell is a sialidase catalytic domain protein. As used herein a “sialidase catalytic domain protein” comprises a catalytic domain of a sialidase but does not comprise the entire amino acid sequence of the sialidase from which the catalytic domain is derived. A “sialidase catalytic domain protein” has sialidase activity, and the term as used herein is interchangeable with a “sialidase” in certain situations. In some embodiments, a sialidase catalytic domain protein comprises at least 10%, at least 20%, at least 50%, at least 70% of the activity of the sialidase from which the catalytic domain sequence is derived. In some embodiments, a sialidase catalytic domain protein comprises at least 90% of the activity of the sialidase from which the catalytic domain sequence is derived.

A sialidase catalytic domain protein can include other amino acid sequences, such as but not limited to additional sialidase sequences, sequences derived from other proteins, or sequences that are not derived from sequences of naturally occurring proteins. Additional amino acid sequences can perform any of a number of functions, including contributing other activities to the catalytic domain protein, enhancing the expression, processing, folding, or stability of the sialidase catalytic domain protein, or even providing a desirable size or spacing of the protein.

In some embodiments, the sialidase catalytic domain protein is a protein that comprises the catalytic domain of the A. viscosus sialidase. In some embodiments, an A. viscosus sialidase catalytic domain protein comprises amino acids 270-666 of the A. viscosus sialidase sequence (SEQ ID NO: 26; GenBank WP_003789074). In some embodiments, an A. Viscosus sialidase catalytic domain protein comprises an amino acid sequence that begins at any of the amino acids from amino acid 270 to amino acid 290 of the A. viscosus sialidase sequence (SEQ ID NO: 26) and ends at any of the amino acids from amino acid 665 to amino acid 901 of said A. viscosus sialidase sequence (SEQ ID NO: 26), and lacks any A. viscosus sialidase protein sequence extending from amino acid 1 to amino acid 269.

In some embodiments, an A. viscosus sialidase catalytic domain protein comprises amino acids 274-681 of the A. viscosus sialidase sequence (SEQ ID NO: 26) and lacks other A. viscosus sialidase sequence. In some embodiments, an A. viscosus sialidase catalytic domain protein comprises amino acids 274-666 of the A. viscosus sialidase sequence (SEQ ID NO: 26) and lacks any other A. viscosus sialidase sequence. In some embodiments, an A. viscosus sialidase catalytic domain protein comprises amino acids 290-666 of the A. viscosus sialidase sequence (SEQ ID NO: 26) and lacks any other A. viscosus sialidase sequence. In yet other embodiments, an A. viscosus sialidase catalytic domain protein comprises amino acids 290-681 of the A. viscosus sialidase sequence (SEQ ID NO: 26) and lacks any other A. viscosus sialidase sequence.

Useful sialidase polypeptides for expression by an engineered immune cell include polypeptides comprising a sequence that is at least 60%, at least 65%, at least 70%, at least 75%, at least, at least 80%, or at least 95%, 96%, 97%, 98%, 99% or 100% identical to SEQ ID NO: 27 or comprises 375, 376, 377, 380, 381, 382, 383, 384, 385, 386, 387, 388, 389, 390, 391, or 392 contiguous amino acids of SEQ ID NO: 27.

In some embodiments, the sialidase is DAS181, a functional derivative thereof (e.g., a fragment thereof), or a biosimilar thereof. In some embodiments, the sialidase comprises an amino acid sequence that is at least about 60% (e.g., at least about any one of 61%, 62%, 63%, 64%, 65%, 66%, 67%, 68%, 69%, or 70%) identical to SEQ ID NO: 2. In some embodiments, the sialidase comprises an amino acid sequence that is at least about 80% (e.g., at least about any one of 85%, 86%, 87%, 88%, 89%, 90%, 91%, 92%, 93%, 94%, 95%, 96%, 97%, 98%, or 99%) or 100% identical to SEQ ID NO: 2. In some embodiments, the sialidase comprises 414, 413, 412, 411, or 410 contiguous amino acids of SEQ ID NO: 2. In some embodiments, the sialidase comprises a fragment of DAS181 without the anchoring domain (AR domain). In some embodiments, the sialidase comprises an amino acid sequence that is at least about 60% (e.g., at least about any one of 61%, 62%, 63%, 64%, 65%, 66%, 67%, 68%, 69%, or 70%) identical to SEQ ID NO: 27. In some embodiments, the sialidase comprises an amino acid sequence that is at least about 80% (e.g., at least about any one of 85%, 86%, 87%, 88%, 89%, 90%, 91%, 92%, 93%, 94%, 95%, 96%, 97%, 98%, or 99%) or 100% identical to SEQ ID NO: 27.

DAS181 is a recombinant sialidase fusion protein with a heparin-binding anchoring domain. DAS181 and methods for preparing and formulating DAS181 are described in U.S. Pat. Nos. 7,645,448; 9,700,602 and 10,351,828, each of which is herein incorporated by reference in their entirety for any and all purposes.

In some embodiments, the sialidase is a secreted form of DAS181, a functional derivative thereof, or a biosimilar thereof. In some embodiments, the secreted DAS181 is membrane-associated via its anchoring domain (AR domain). In some embodiments, the heterologous nucleotide sequence encoding a secreted form of DAS181 encodes a secretion sequence operably linked to DAS181, wherein the secretion sequence enables secretion of the DAS181 from eukaryotic cells. In some embodiments, the sialidase comprises an amino acid sequence that is at least about 60% (e.g., at least about any one of 61%, 62%, 63%, 64%, 65%, 66%, 67%, 68%, 69%, or 70%) identical to SEQ ID NO: 28. In some embodiments, the sialidase comprises an amino acid sequence that is at least about 80% (e.g., at least about any one of 85%, 86%, 87%, 88%, 89%, 90%, 91%, 92%, 93%, 94%, 95%, 96%, 97%, 98%, or 99%) or 100% identical to SEQ ID NO: 28. In some embodiments, the sialidase comprises 414, 413, 412, 411, or 410 contiguous amino acids of SEQ ID NO: 28. An exemplary secreted form of DAS181 and its activity is described in Example 2.

In some embodiments, the sialidase is a transmembrane form of DAS181, a functional derivative thereof, or a biosimilar thereof. In some embodiments, the sialidase comprises an amino acid sequence that is at least about 60% (e.g., at least about any one of 61%, 62%, 63%, 64%, 65%, 66%, 67%, 68%, 69%, or 70%) identical to SEQ ID NO: 31. In some embodiments, the sialidase comprises an amino acid sequence that is at least about 80% (e.g., at least about any one of 85%, 86%, 87%, 88%, 89%, 90%, 91%, 92%, 93%, 94%, 95%, 96%, 97%, 98%, or 99%) or 100% identical to SEQ ID NO: 31. In some embodiments, the sialidase comprises 414, 413, 412, 411, or 410 contiguous amino acids of SEQ ID NO: 31. An exemplary transmembrane form of DAS181 and its activity is described in Example 2.

In some embodiments, the sialidase cleaves sialylated glycans regardless of the structure of the more distant parts of the oligosaccharide chain (e.g. α2,3 vs. α2,6 linkage, chain length, or modification). In some embodiments, the sialidase is capable of cleaving glycans with Neu5Ac alpha(2,6)-Gal sialidase or Neu5Ac alpha(2,3)-Gal terminal sialic acid structures. In some embodiments, the sialidase is capable of cleaving glycans with Neu5Ac alpha(2,6)-Gal sialidase or Neu5Ac alpha(2,3)-Gal terminal sialic acid structures with near complete removal at low concentrations (e.g., 0.5 nM). In some embodiments, the sialidase is capable of cleaving glycans with Neu5Ac alpha(2,6)-Gal sialidase or Neu5Ac alpha(2,3)-Gal terminal sialic acid structures by at least 85% (e.g., at least 86%, 87%, 88%, or 89%) or at least 90% (e.g., at least 91%, 92%, 93%, 94%, 95%, 96%, 97%, or 98%) at low concentrations (e.g., 0.5 nM). In some embodiments, the sialidase is further capable of cleaving glycans with KDN terminal sialic acid structure (2-keto-3-deoxynononic acid). In some embodiments, the sialidase is capable of cleaving glycans with KDN terminal sialic acid structure. In some embodiments, the sialidase is capable of near complete removal sialic acids from glycans with Neu5Ac alpha(2,6)-Gal sialidase, Neu5Ac alpha(2,3)-Gal, or KDN terminal sialic acid structures at concentrations of between 5 nM and 50 nM. In some embodiments, the sialidase is capable of cleaving glycans with Neu5Ac alpha(2,6)-Gal sialidase, Neu5Ac alpha(2,3)-Gal, or KDN terminal sialic acid structures by at least 85% (e.g., at least 86%, 87%, 88%, or 89%) or at least 90% (e.g., at least 91%, 92%, 93%, 94%, 95%, 96%, 97%, 98%, or 99%) at concentrations between 5 nM and 50 nM (e.g., 5-10 nM, 10-15 nM, 15-20 nM, 20-25 nM, 25-30 nM, 35-40 nM, 40-45 nM, or 45-50 nM). In some embodiments, the sialidase is capable of efficiently cleaving sialic acid residues with internal sulfate and fucosyl groups (e.g., at least 86%, 87%, 88%, or 89%, 90%, 91%, 92%, 93%, 94%, 95%, 96%, 97%, or 98% sialic acid removal). Example 1 provides results demonstrating the unexpectedly broad activity and potency of an exemplary sialidase (DAS181) derived from an Actinomyces viscosus sialidase.

TABLE 2
Engineered Sialidases

| Name | Sequence |
|---|---|
| A. viscosus | mtshspfsrr hlpallgslp laatgliaaa ppahavptsd gladvtitqv |
| sialidase | napadglysv gdvmtfnitl tntsgeahsy apastnlsgn vskerwrnvp |
| | agttktdctg lathtvtaed lkaggftpqi ayevkaveya gkalstpeti |
| | kgatspvkan slrvesitps sskeyyklgd tvtytvrvrs vsdktinvaa |
| | tessfddlgr qchwgglkpg kgavynckpl thtitqadvd agrwtpsitl |
| | tatgtdgtal qtltatgnpi nvvgdhpqat papapdaste 1pasmsqaqh |
| | vapntatdny ripaittapn gdllisyder pkdngnggsd apnpnhivqr |
| | rstdggktws aptyihqgte tgkkvgysdp syvvdhqtgt ifnfhvksyd |
| | hgwgnsqagt dpenrgiiqa evststdngw twthrtitad itkdnpwtar |
| | faasgqgigi qhgphagrlv qqytirtagg avqavsvysd dhgktwqagt |
| | pvgtgmdenk vvelsdgslm Insrasdssg frkvahstdg gqtwsepvsd |
| | knlpdsvdna qiirafpnaa pddprakvll 1shspnpkpw srdrgtisms |
| | cddgaswtts kvfhepfvgy ttiavqsdgs igllsedahd ganyggiwyr |
| | nftmnwlgeq cgqkpaepsp apsptaapsa apseqpapsa apsteptqap |
| | apssapepsa vpepssapap epttapstep tptpapssap epsagptaap |
| | apetssapaa eptqaptvap saeptqvpga qpsaapsekp gaqpssapkp |
| | datgrapsvv npkataapsg kasssaspap srsatatskp gmepdeidrp |
| | sdgamaqptg gasapsaapt qaakagsrls rtgtnallvl glagvavvgg |
| | ylllrarrsk n (SEQ ID NO: 26) |
| AvCD | MGDHPQATPA PAPDASTELP ASMSQAQHLA ANTATDNYRI PAITTAPNGD |
| | LLISYDERPK DNGNGGSDAP NPNHIVQRRS TDGGKTWSAP TYIHQGTETG |
| | KKVGYSDPSY VVDHQTGTIF NFHVKSYDQG WGGSRGGTDP ENRGIIQAEV |
| | STSTDNGWTW THRTITADIT KDKPWTARFA ASGQGIQIQH GPHAGRLVQQ |
| | YTIRTAGGAV QAVSVYSDDH GKTWQAGTPI GTGMDENKVV ELSDGSLMLN |
| | SRASDGSGFR KVAHSTDGGQ TWSEPVSDKN LPDSVDNAQI IRAFPNAAPD |
| | DPRAKVLLLS HSPNPRPWSR DRGTISMSCD DGASWTTSKV FHEPFVGYTT |
| | IAVQSDGSIG LLSEDAHNGA DYGGIWYRNF TMNWLGEQCG QKPAE |
| | (SEQ ID NO: 1) |
| DAS181 | MGDHPQATPA PAPDASTELP ASMSQAQHLA ANTATDNYRI PAITTAPNGD |
| | LLISYDERPK DNGNGGSDAP NPNHIVQRRS TDGGKTWSAP TYIHQGTETG |
| | KKVGYSDPSY VVDHQTGTIF NFHVKSYDQG WGGSRGGTDP ENRGIIQAEV |
| | STSTDNGWTW THRTITADIT KDKPWTARFA ASGQGIQIQH GPHAGRLVQQ |
| | YTIRTAGGAV QAVSVYSDDH GKTWQAGTPI GTGMDENKVV ELSDGSLMLN |
| | SRASDGSGFR KVAHSTDGGQ TWSEPVSDKN LPDSVDNAQI IRAFPNAAPD |
| | DPRAKVLLLS HSPNPRPWSR DRGTISMSCD DGASWTTSKV FHEPFVGYTT |
| | IAVQSDGSIG LLSEDAHNGA DYGGIWYRNF TMNWLGEQCG QKPAKRKKKG |
| | GKNGKNRRNR KKKNP (SEQ ID NO: 2) |
| DAS181 | GDHPQATPAP APDASTELPA SMSQAQHLAA NTATDNYRIP AITTAPNGDL |
| without | LISYDERPKD NGNGGSDAPN PNHIVQRRST DGGKTWSAPT YIHQGTETGK |
| initial Met | KVGYSDPSYV VDHQTGTIFN FHVKSYDQGW GGSRGGTDPE NRGIIQAEVS |
| and without | TSTDNGWTWT HRTITADITK DKPWTARFAA SGQGIQIQHG PHAGRLVQQY |
| anchoring | TIRTAGGAVQ AVSVYSDDHG KTWQAGTPIG TGMDENKVVE LSDGSLMLNS |
| domain | RASDGSGFRK VAHSTDGGQT WSEPVSDKNL PDSVDNAQII RAFPNAAPDD |
| | PRAKVLLLSH SPNPRPWSRD RGTISMSCDD GASWTTSKVF HEPFVGYTTI |
| | AVQSDGSIGL LSEDAHNGAD YGGIWYRNFT MNWLGEQCGQ KPA |
| | (SEQ ID NO: 27) |
| Membrane | METDTLLLWVLLLWVPGSTGDMGDHPQATPAPAPDASTELPASMSQAQHLAANTATD |
| Bound | NYRIPAITTAPNGDLLISYDERPKDNGNGGSDAPNPNHIVQRRSTDGGKTWSAPTYI |
| sialidase | HQGTETGKKVGYSDPSYVVDHQTGTIFNFHVKSYDQGWGGSRGGTDPENRGIIQAEV |
| (secretion | STSTDNGWTWTHRTITADITKDKPWTARFAASGQGIQIQHGPHAGRLVQQYTIRTAG |
| signal and | GAVQAVSVYSDDHGKTWQAGTPIGTGMDENKVVELSDGSLMLNSRASDGSGFRKVAH |
| TM | STDGGQTWSEPVSDKNLPDSVDNAQIIRAFPNAAPDDPRAKVLLLSHSPNPRPWSRD |
| underlined) | RGTISMSCDDGASWTTSKVFHEPFVGFTTIAVQSDGSIGLLSEDAHNGADYGGIWYR |
| | NFTMNWLGEQCGQKPANAVGQDTQEVIVVPHSLPFKVVVISAILALVVLTIISLIIL |
| | IMLWQKKPR (SEQ ID NO: 31) |
| Secreted | METDTLLLWVLLLWVPGSTGDGDHPQATPAPAPDASTELPASMSQAQHLAANTATDNYRIPAI |
| sialidase | TTAPNGDLLISYDERPKDNGNGGSDAPNPNHIVQRRSTDGGKTWSAPTYIHQGTETGKKVGYS |
| (secretion | DPSYVVDHQTGTIFNFHVKSYDQGWGGSRGGTDPENRGIIQAEVSTSTDNGWTWTHRTITADI |
| signal | TKDKPWTARFAASGQGIQIQHGPHAGRLVQQYTIRTAGGAVQAVSVYSDDHGKTWQAGTPIGT |
| underlined) | GMDENKVVELSDGSLMLNSRASDGSGFRKVAHSTDGGQTWSEPVSDKNLPDSVDNAQIIRAFP |
| | NAAPDDPRAKVLLLSHSPNPRPWSRDRGTISMSCDDGASWTTSKVFHEPFVGYTTIAVQSDGS |
| | IGLLSEDAHNGADYGGIWYRNFTMNWLGEQCGQKPAKRKKKGGKNGKNRRNRKKKNP |
| | (SEQ ID NO: 28) |

TABLE 3
Human Sialidases

| Name | Uniprot Identifier | SEQ ID NO |
|---|---|---|
| Human Neu 1 | Q99519 | 3 |
| Human Neu 2 | Q9Y3R4 | 4 |
| Human Neu 3 | Q9UQ49 | 5 |
| Human Neu 4 | Q8WWR8 | 6 |
| Human Neu 4 Isoform 2 | Q8WWR8 | 7 |
| Human Neu 4 Isoform 3 | Q8WWR8 | 8 |

TABLE 4
Sialidases in organisms that are largely commensal with humans

| | Uniprot/ | Gene | SEQ |
|---|---|---|---|
| Organism | Genbank ID | name | ID NO |
| Actinomyces viscosus | Q59164 | nanH | 9 |
| Actinomyces viscosus | A0A448PLN7 | nanA | 10 |
| Streptococcus oralis | A0A081R4G6 | nanA | 11 |
| Streptococcus oralis | D4FUA3 | nanH | 12 |
| Streptococcus mitis | A0A081Q0I6 | nanA | 13 |
| Streptococcus mitis | A0A3R9LET9 | nanA_1 | 14 |
| Streptococcus mitis | A0A3R9J1C3 | nanA_2 | 15 |
| Streptococcus mitis | A0A3R9IIK2 | nanA_3 | 16 |
| Streptococcus mitis | A0A3R9IXG7 | nanA_4 | 17 |
| Streptococcus mitis | A0A3R9K5C5 | nanA_5 | 18 |
| Streptococcus mitis | J1H2U0 | nanH | 19 |
| Porphyromonas gingivalis | B2RL82 | | 20 |
| Tannerella forsythia | Q84BM9 | siaHI | 21 |
| Tannerella forsythia | A0A1D3USB1 | nanH | 22 |
| Akkermansia Muciniphila | B2UPI5 | | 23 |
| Akkermansia Muciniphila | B2UN42 | | 24 |
| Bacteroides thetaiotaomicron | Q8AAK9 | | 25 |

TABLE 5
Additional sialidases

| Organism | Uniprot/Genbank ID |
|---|---|
| Actinotignum schaalii | S2VK03 |
| Anaerotruncus colihominis | B0PE27 |
| Ruminococcus gnavus | A0A2N5NZH2 |
| Clostridium difficile | Q185B3 |
| Clostridium septicum | P29767 |
| Clostridium perfringens | P10481 |
| Clostridium perfringens | Q8XMY5 |
| Clostridium perfringens | A0A2Z3TZA2 |
| Vibrio cholerae | P0C6E9 |
| Salmonella typhimurium | P29768 |
| Paeniclostridium sordellii | A0A446I8A2 |
| Streptococcus pneumoniae (NanA) | P62576 |
| Streptococcus pneumoniae (NanB) | Q54727 |
| Pseudomonas aeruginosa | A0A2X4HZU8 |
| Aspergillus fumigatus | Q4WQS0 |
| Arthrobacter ureafaciens | Q5W7Q2 |
| Micromonospora viridifaciens | Q02834 |

### Anchoring Domain

In some embodiments, the sialidase comprises an anchoring domain. As used herein, an “extracellular anchoring domain” or “anchoring domain” is any moiety that interacts with an entity that is at or on the exterior surface of a target cell or is in close proximity to the exterior surface of a target cell. An anchoring domain can serve to retain a sialidase of the present disclosure at or near the external surface of a target cell. An extracellular anchoring domain may bind 1) a molecule expressed on the surface of a cancer cell, or a moiety, domain, or epitope of a molecule expressed on the surface of a cancer cell, 2) a chemical entity attached to a molecule expressed on the surface of a cancer cell, or 3) a molecule of the extracellular matrix surrounding a cancer cell.

An exemplary anchoring domain binds to heparin/sulfate, a type of GAG that is ubiquitously present on cell membranes. Many proteins specifically bind to heparin/heparan sulfate, and the GAG-binding sequences in these proteins have been identified (Meyer, F A, King, M and Gelman, R A. (1975) Biochimica et Biophysica Acta 392: 223-232; Schauer, S. ed., pp 233. Sialic Acids Chemistry, Metabolism and Function. Springer-Verlag, 1982). For example, the GAG-binding sequences of human platelet factor 4 (PF4) (SEQ ID NO:66), human interleukin 8 (IL8)(SEQ ID NO:67), human antithrombin III (AT III) (SEQ ID NO:68), human apoprotein E (ApoE) (SEQ ID NO:69), human angio-associated migratory cell protein (AAMP) (SEQ ID NO:70), or human amphiregulin (SEQ ID NO:71) have been shown to have very high affinity to heparin.

In some embodiments, the anchoring domain is anon-protein anchoring moiety, such as a phosphatidylinositol (GPI) linker.

In some embodiments, the anchoring domain is positively charged at physiological pH. In some embodiments, the anchoring domain comprises at least 4, 5, 6, 7, 8, 9, 10, or more positively charged amino acid residues, wherein lysine or arginine are counted as positively charged residues. In some embodiments, the anchoring domain comprises at least 20% (e.g., at least 25%, 30%, 35%, 40%, or 45%) positive residues within the anchoring domain sequence. For example, the sequences of positively charged heparin-binding domains are shown in Table 6.

TABLE 6
Sequence comparison of heparin-
binding domains.

| SEQ | | | Posi- | % |
|---|---|---|---|---|
| ID | | | tive/ | Posi- |
| NO. | Protein | Sequence | Total* | tive |
| 66 | PF4 | ⁴⁷NGRRICLDLQAPL | 6/24 | 25% |
| | | IYKKIKKLLES⁷⁰ | | |
| 67 | IL-8 | ⁴⁶GRELCLDPKENWV | 6/27 | 22% |
| | | QRVVEKFLKRAENS⁷² | | |
| 68 | ATIII | ¹¹⁸QIHFFFAKLNCR | 8/34 | 24% |
| | | LYRKANKSSKLVSA | | |
| | | NRLFGDKS¹⁵¹ | | |
| 69 | ApoE | ¹³²ELRVRLASHLRKL | 10/34 | 29% |
| | | RKRLLRDADDLQKRL | | |
| | | AVYQAG¹⁶⁵ | | |
| 70 | AAMP | ¹⁴RRLRRMESESES²⁵ | 4/21 | 33% |
| 71 | Amphiregulin* | ²⁵KRKKKGGKNGKN | 10/21 | 48% |
| | | TTNTKKKNP⁴⁵ | | |

*Lysine or arginine amino acid residues are counted as positive.

### Linkers

A sialidase that includes a sialidase catalytic domain and other non-sialidase domains can optionally include one or more polypeptide linkers that can join various domains of the sialidase. Linkers can be used to provide optimal spacing or folding of the domains of a sialidase. The domains of a sialidase joined by linkers can be sialidase domains, anchoring domains, transmembrane domains, or any other domains or moieties of the sialidase that provide additional functions such as enhancing protein stability, facilitating purification, etc. Some preferred linkers include the amino acid glycine. In a non-limiting example, a flexible linker can be a linker having the sequence: (GGGGS (SEQ ID NO: 55))n, where n is 1-20. In some embodiments, the linker is a hinge region of an immunoglobulin. Any hinge or linker sequence capable of keeping the catalytic domain free of steric hindrance can be used to link a domain of a sialidase to another domain (e.g., a transmembrane domain or an anchoring domain. In some embodiments, the linker is a hinge domain comprising the sequence of SEQ ID NO: 62.

### Secretion Sequence

In some embodiments, the heterologous nucleotide sequence encoding the sialidase further encodes a secretion sequence (e.g., a signal sequence or signal peptide) operably linked to the sialidase. The terms “secretion sequence,” “signal sequence,” and “signal peptide” are used interchangeably. In some embodiments, the secretion sequence is a signal peptide operably linked to the N-terminus of the sialidase. In some embodiments, the length of the secretion sequence ranges between 15 and 30 amino acids (e.g., between 15 and 25 amino acids, between 15 and 22 amino acids, or between 20 and 25 amino acids). In some embodiments, the secretion sequence enables secretion of the sialidase from eukaryotic cells. During translocation across the endoplasmic reticulum membrane, the secretion sequence is usually cleaved off and the protein (e.g., sialidase) enters the secretory pathway. In some embodiments, the heterologous nucleotide sequence encodes, from N-terminus to C-terminus, a secretion sequence, a sialidase, and a transmembrane domain, wherein the sialidase is operably linked to the secretion sequence and the transmembrane domain. In some embodiments, the N-terminal secretion sequence is cleaved resulting in a sialidase with an N-terminal extracellular domain. An exemplary secretion sequence is provided in SEQ ID NO: 40.

### Transmembrane Domain

In some embodiments, the sialidase comprises a transmembrane domain. In some embodiments, the sialidase domain can be joined to a mammalian (preferably human) transmembrane (TM) domain. This arrangement permits the sialidase to be expressed on the cell surface. Suitable transmembrane domain include, but are not limited to a sequence comprising human CD28 TM domain (NM_006139; FWVLVVVGGVLACYSLLVTVAFIIFWV (SEQ ID NO: 46), human CD4 TM domain (M35160; MALIVLGGVAGLLLFIGLGIFF (SEQ ID NO: 47); human CD8 TM1 domain (NM_001768; IYIWAPLAGTCGVLLLSLVIT (SEQ ID NO: 48); human CD8 TM2 domain (NM_001768; IYIWAPLAGTCGVLLLSLVITLY (SEQ ID NO: 49); human CD8 TM3 domain (NM_001768; IYIWAPLAGTCGVLLLSLVITLYC (SEQ ID NO: 50); human 41BB TM domain (NM_001561; IISFFLALTSTALLFLLFF LTLRFSVV (SEQ ID NO: 51); human PDGFR TM1 domain (VVISAILA LVVLTIISLIILI; SEQ ID NO:52); and human PDGFR TM2 domain NAVGQDTQEVIVVPHSLPFKVVVISAILALVVLTIISLIILIMLWQKKPR; SEQ ID NO: 45)

In some embodiments, the heterologous nucleotide sequence encoding a sialidase encodes a protein comprising, from amino terminus to carboxy terminus, a secretion sequence (e.g., SEQ ID NO: 40), a sialidase (e.g., a sialidase comprising an amino acid sequence selected from SEQ ID NOs: 1-27, and a transmembrane domain (e.g., a transmembrane domain selected from SEQ ID NOs: 45-52). However, any suitable secretion sequence, sialidase domain sequence, or transmembrane domain may be used. In some embodiments, the heterologous nucleotide sequence encoding a sialidase encodes a protein comprising, from amino terminus to carboxy terminus, a secretion sequence (e.g., SEQ ID NO: 40), the sialidase of SEQ ID NO: 27, and a transmembrane domain (e.g., a transmembrane domain selected from SEQ ID NOs: 45-52).

In some embodiments, the sialidase has at least 80% (e.g., at least about any one of 85%, 86%, 87%, 88%, 89%) or at least 90% (e.g., at least about any one of 91%, 92%, 94%, 96%, 98%, or 99%) sequence identity to a sequence selected from SEQ ID NOs: 31-33. In some embodiments, the sialidase comprises a sequence selected from SEQ ID NOs: 31-33. In some embodiments, the sialidase comprises the amino acid sequence of SEQ ID NO: 31.

In some embodiments, the transmembrane domain is fused to a sialidase catalytic domain via a linker such as a hinge region or another peptide linker. In some embodiments, the transmembrane domain is fused to a sialidase catalytic domain directly, without a linker.

### 2. Engineered Immune Cells

The present application provides compositions comprising engineered immune cells for treating a cancer in an individual in need thereof. In some embodiments, the present application provides an engineered immune cell for treating a cancer in an individual in need thereof, wherein the engineered immune cell comprises a first heterologous nucleotide sequence encoding a sialidase and a second heterologous nucleotide sequence encoding a chimeric immune receptor. In some embodiments, the engineered immune cell is a cytotoxic T cell, a helper T cell, a suppressor T cell, a natural killer (NK) cell, a macrophage, or a natural killer T (NKT) cell.

In some embodiments, the engineered immune cell is a T-cell. In some embodiments, the engineered immune cell is a NK cell. In some embodiments, the engineered immune cell is an NKT cell. In some embodiments, the first heterologous nucleotide sequence and the second heterologous nucleotide sequence are operably linked to the same promoter. In some embodiments, the first heterologous nucleotide sequence and the second heterologous nucleotide sequence are operably linked to different promoters.

In some embodiments, the present application provides a composition comprising an engineered immune cell comprising a first heterologous nucleotide sequence encoding a sialidase, and a second engineered immune cell comprising a second heterologous nucleotide sequence encoding a chimeric immune receptor. In some embodiments, the first engineered immune cell is a T-cell, a natural killer (NK) cell, a macrophage, or a natural killer T (NKT) cell and the second engineered immune cell is a T-cell, a natural killer (NK) cell, a macrophage, or a natural killer T (NKT) cell. In some embodiments, the first and the second engineered immune cells are the same type of cell. In some embodiments, the first and second engineered immune cells are T cells. In some embodiments, the first and second engineered immune cells are NK cells. In some embodiments, the first and the second engineered immune cell are different types of cells.

### Chimeric Antigen Receptor (CAR)

“Chimeric antigen receptor” or “CAR” as used herein refers to an engineered receptor that can be used to graft one or more target-binding specificities onto an immune cell, such as T cells or NK cells. In some embodiments, the chimeric antigen receptor comprises an extracellular target binding domain, a transmembrane domain, and an intracellular signaling domain of a T cell receptor and/or other receptors.

Some embodiments of the engineered immune cells described herein comprise a chimeric antigen receptor (CAR). In some embodiments, the CAR comprises an antigen-binding moiety and an effector protein or fragment thereof comprising a primary immune cell signaling molecule or a primary immune cell signaling domain that activates the immune cell expressing the CAR directly or indirectly. In some embodiments, the CAR comprises an antigen-binding domain, a transmembrane domain, and an intracellular signaling domain. Also provided an engineered immune cells (e.g., T cell or NK cell) comprising the CAR. The antigen-binding moiety and the effector protein or fragment thereof may be present in one or more polypeptide chains. Exemplary CAR constructs have been described, for example, in U.S. Pat. No. 9,765,342B2, WO2002/077029, and WO2015/142675, which are hereby incorporated by reference. Any one of the known CAR constructs may be used in the present application.

In some embodiments, the primary immune cell signaling molecule or primary immune cell signaling domain comprises an intracellular domain of a molecule selected from the group consisting of CD3ζ, FcRγ, FcRβ, CD3γ, CD3δ, CD3ε, CD5, CD22, CD79a, CD79b, and CD66d. In some embodiments, the intracellular signaling domain consists of or consists essentially of a primary immune cell signaling domain. In some embodiments, the intracellular signaling domain comprises an intracellular signaling domain of CD3ζ. In some embodiments, the CAR further comprises a costimulatory molecule or fragment thereof. In some embodiments, the costimulatory molecule or fragment thereof is derived from a molecule selected from the group consisting of CD27, CD28, 4-1BB, OX40, CD30, CD40, PD-1, ICOS, LFA-1, CD2, CD7, LIGHT, NKG2C, B7-H3, and a ligand that specifically binds CD83. In some embodiments, the intracellular signaling domain further comprises a co-stimulatory domain comprising a CD28 intracellular signaling sequence. In some embodiments, the intracellular signaling domain comprises a CD28 intracellular signaling sequence and an intracellular signaling sequence of CD3ζ.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the CD28, CD3E, CD3ζ, CD45, CD4, CD5, CD8, CD9, CD16, CD22, CD33, CD37, CD64, CD80, CD86, CD134, CD137, or CD154. In some embodiments, the CAR is a CD-19 CAR comprising including CD19 scFv from clone FMC63 (Nicholson I C, et al. Mol Immunol. 1997), a CH2-CH3 spacer, a CD28-TM, 41BB, and CD3ζ. In some embodiments, the transmembrane domain may be synthetic, in which case it may comprise predominantly hydrophobic residues such as leucine and valine. In some embodiments, a triplet of phenylalanine, tryptophan and valine may be found at each end of a synthetic transmembrane domain. In some embodiments, a short oligo- or polypeptide linker, having a length of, for example, between about 2 and about 10 (such as about any of 2, 3, 4, 5, 6, 7, 8, 9, or 10) amino acids in length may form the linkage between the transmembrane domain and the intracellular signaling domain. In some embodiments, the linker is a glycine-serine doublet.

In some embodiments, the transmembrane domain that is naturally associated with one of the sequences in the intracellular domain is used (e.g., if an intracellular domain comprises a CD28 co-stimulatory sequence, the transmembrane domain is derived from the CD28 transmembrane domain). In some embodiments, the transmembrane domain can be selected or modified by amino acid substitution to avoid binding of such domains to the transmembrane domains of the same or different surface membrane proteins to minimize interactions with other members of the receptor complex.

The intracellular signaling domain of the CAR is responsible for activation of at least one of the normal effector functions of the immune cell in which the CAR has been placed in. Effector function of a T cell, for example, may be cytolytic activity or helper activity including the secretion of cytokines. Thus, the term “intracellular signaling domain” refers to the portion of a protein, which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term “intracellular signaling sequence” is thus meant to include any truncated portion of the intracellular signaling domain sufficient to transduce the effector function signal.

Examples of intracellular signaling domains for use in the CAR of the present application include the cytoplasmic sequences of the TCR and co-receptors that act in concert to initiate signal transduction following antigen receptor engagement, as well as any derivative or variant of these sequences and any synthetic sequence that has the same functional capability.

It is known that signals generated through the TCR alone may be insufficient for full activation of the T cell and that a secondary or co-stimulatory signal may also be required. Thus, T cell activation can be said to be mediated by two distinct classes of intracellular signaling sequence: those that initiate antigen-dependent primary activation through the TCR (primary signaling sequences) and those that act in an antigen-independent manner to provide a secondary or co-stimulatory signal (co-stimulatory signaling sequences).

Primary signaling sequences regulate primary activation of the TCR complex either in a stimulatory way, or in an inhibitory way. Primary signaling sequences that act in a stimulatory manner may contain signaling motifs, which are known as immunoreceptor tyrosine-based activation motifs or ITAMs. The CAR constructs in some embodiments comprise one or more ITAMs. Examples of ITAM containing primary signaling sequences that are of particular use in the invention include those derived from CD3ζ, FcRγ, FcRβ, CD3γ, CD36, CD3ε, CD5, CD22, CD79a, CD79b, and CD66d.

In some embodiments, the CAR comprises a primary signaling sequence derived from CD3ζ. For example, the intracellular signaling domain of the CAR can comprise the CD3ζ intracellular signaling sequence by itself or combined with any other desired intracellular signaling sequence(s) useful in the context of the CAR described herein. For example, the intracellular domain of the CAR can comprise a CD3ζ intracellular signaling sequence and a costimulatory signaling sequence. The costimulatory signaling sequence can be a portion of the intracellular domain of a costimulatory molecule including, for example, CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, and the like.

In some embodiments, the intracellular signaling domain of the CAR comprises the intracellular signaling sequence of CD3ζ and the intracellular signaling sequence of CD28. In some embodiments, the intracellular signaling domain of the CAR comprises the intracellular signaling sequence of CD3ζ and the intracellular signaling sequence of 4-1BB. In some embodiments, the intracellular signaling domain of the CAR comprises the intracellular signaling sequence of CD3ζ and the intracellular signaling sequences of CD28 and 4-1BB. In some embodiments, the antigen binding moiety comprises an scFv or a Fab. In some embodiments, the antigen binding moiety is targeted to an tumor-associated or tumor-specific antigen, such as, without limitation: carcinoembryonic antigen, alphafetoprotein, MUC16, survivin, glypican-3, B7 family members, LILRB, CD19, BCMA, NY-ESO-1, CD20, CD22, CD33, CD38, CEA, EGFR (such as EGFRvIII), GD2, HER2, IGF1R, mesothelin, PSMA, ROR1, WT1, NY-ESO-1, CDH17, and other tumor antigens with clinical significance.

Also provided herein are engineered immune cells (such as lymphocytes, e.g., T cells, NK cells, or macrophages) expressing any one of the CARs and sialidases described herein. Also provided is a method of producing an engineered immune cell expressing any one of the CARs and sialidases described herein, the method comprising introducing one or more vector(s) comprising a nucleic acid encoding the CAR and/or sialidase into the immune cell. In some embodiments, the CAR and sialidase are encoded on the same vector. In some embodiments, the CAR and sialidase are encoded by different vectors. In some embodiments, introducing the vector(s) into the immune cell comprises transducing the immune cell with the vector. In some embodiments, the vector is a lentiviral vector. In some embodiments, introducing the vector into the immune cell comprises transfecting the immune cell with the vector. Transduction or transfection of the vector into the immune cell can be carried about using any method known in the art.

### Engineered T Cell Receptor

In some embodiments, the chimeric receptor is a T cell receptor. In some embodiments, wherein the engineered immune cell is a T cell, the T cell receptor is an endogenous T cell receptor. In some embodiments, the engineered immune cell with the TCR is pre-selected. In some embodiments, the T cell receptor is a recombinant TCR. In some embodiments, the TCR is specific for a tumor antigen. In some embodiments, the tumor antigen is selected from carcinoembryonic antigen, alphafetoprotein, MUC16, survivin, glypican-3, B7 family members, LILRB, CD19, BCMA, NY-ESO-1, CD20, CD22, CD33, CD38, CEA, EGFR (such as EGFRvIII), GD2, HER2, IGF1R, mesothelin, PSMA, ROR1, WT1, NY-ESO-1, CDH17, and other tumor antigens with clinical significance. In some embodiments, the tumor antigen is derived from an intracellular protein of tumor cells. Many TCRs specific for tumor antigens (including tumor-associated antigens) have been described, including, for example, NY-ESO-1 cancer-testis antigen, the p53 tumor suppressor antigens, TCRs for tumor antigens in melanoma (e.g., MARTI, gp 100), leukemia (e.g., WT1, minor histocompatibility antigens), and breast cancer (HER2, NY-BR1, for example). Any of the TCRs known in the art may be used in the present application. In some embodiments, the TCR has an enhanced affinity to the tumor antigen. Exemplary TCRs and methods for introducing the TCRs to immune cells have been described, for example, in U.S. Pat. No. 5,830,755, and Kessels et al. Immunotherapy through TCR gene transfer. Nat. Immunol. 2, 957-961 (2001). In some embodiments, the engineered immune cell is a TCR-T cell.

### TCR Fusion Protein (TFP)

In some embodiments, the engineered immune cell comprises a TCR fusion protein (TFP). “TCR fusion protein” or “TFP” as used herein refers to an engineered receptor comprising an extracellular target-binding domain fused to a subunit of the TCR-CD3 complex or a portion thereof, including TCRα chain, TCRβ chain, TCRγ chain, TCRδ chain, CD3ε, CD3δ, or CD3γ. The subunit of the TCR-CD3 complex or portion thereof comprise a transmembrane domain and at least a portion of the intracellular domain of the naturally occurring TCR-CD3 subunit. The TFP comprises the extracellular domain of the TCR-CD3 subunit or a portion thereof.

Exemplary TFP constructs comprising an antibody fragment as the target-binding moiety have been described, for example, in WO2016187349 and WO2018098365, which are hereby incorporated by reference.

### Targeting Sialidases to Tumor-Associated Antigens

Sialidase expressing engineered immune cells (e.g., CAR-T, CAR-NK, CAR-NKT, or CAR-M cells) can be targeted to any of a variety of tumor-associated antigens (TAAs) or immune cell receptors, which may include without limitation: carcinoembryonic antigen, alphafetoprotein, MUC16, survivin, glypican-3, B7 family members, LILRB, CD19, BCMA, NY-ESO-1, CD20, CD22, CD33, CD38, CEA, EGFR (such as EGFRvIII), GD2, HER2, IGF1R, mesothelin, PSMA, ROR1, WT1, NY-ESO-1, CDH17, and other tumor antigens with clinical significance. Engineered immune cells (e.g., CAR-T or CAR-NK cells) can be used to direct sialidases to cancer cells expressing these or any number of known cancer antigens. Engineered immune cells (e.g., CAR-T or CAR-NK cells) expressing sialidase can also be targeted to a variety of immune cells expressing various immune cell antigens, such as, without limitation: CD24, CD200, VSIG-3, RAE-18, MICA/B, ICAM, B7H4, CD155, CDH17, PDL-1, LHRH, LHR, HER, and others.

These sialidase expressing engineered immune cells (e.g., CAR-T or CAR-NK cells) can be delivered to the patient in any way known in the art for delivering engineered immune cells (e.g., CAR-T or CAR-NK cells). Without being bound by theory, sialidase expressed on the surface of or secreted by sialidase expressing engineered immune cells (e.g., CAR-T or CAR-NK cells) remove sialic acids from sialoglycans expressed on immune cells and/or tumor cells, thus allowing immune activation against cancer and combinatory therapeutics to get in the TME. With respect to tumor cells, as they are desialylated, they become exposed to attack by activated NK cells and other immune cells, resulting in reduction in tumor size.

The engineered immune cells (e.g., CAR-T or CAR-NK cells) set forth herein can be engineered to express sialidase, such as, without limitation, DAS181, on the engineered immune cell (e.g., CAR-T or CAR-NK cells) cell surface membrane, such that the sialidase is membrane bound. Without being bound by theory, membrane bound sialidases are not freely circulating and only come into contact with the target cells of the engineered immune cells (e.g., CAR-T or CAR-NK cells), namely tumor cells expressing the antigens that the chimeric immune receptor (e.g., CAR) targets. For example, if the engineered immune cell is an anti-CD-19 receptor expressing CAR-T, then the membrane bound sialidases will primarily only come into contact with tumor cells that express CD-19. In this way, the sialidases will not desialylate non-targeted cells, such as erythrocytes, but will instead eliminate sialic acid primarily only from tumor cells. The engineered immune cells (e.g., CAR-T or CAR-NK cells) set forth herein can also be engineered so that they express secreted sialidase, such as, without limitation, secreted DAS181.

### 3. Third Nucleotide Sequence Encoding a Secreted Heterologous Protein

In some embodiments according to any one of the engineered immune cells or compositions described herein, the engineered immune cell comprises a third nucleotide sequence encoding a heterologous protein. In some embodiments, the heterologous protein is a secreted protein.

In some embodiments, the heterologous protein is an immune checkpoint inhibitor. In some embodiments, the immune checkpoint inhibitor is an inhibitor of CTLA-4, PD-1, PD-L1, B7-H4, or HLA-G. In some embodiments, the immune checkpoint inhibitor is an antibody. In some embodiments, the immune checkpoint modulator is an immune checkpoint inhibitor, such as an inhibitor of PD-1, PD-L1, PD-L2, CD47, CXCR4, CSF1R, LAG-3, TIM-3, HHLA2, BTLA, CD160, CD73, CTLA-4, B7-H4, TIGIT, VISTA, or 2B4. In some embodiments, the immune checkpoint modulator is an inhibitor of PD-1. In some embodiments, the immune checkpoint inhibitor is an antibody against an immune checkpoint molecule, such as an anti-PD-1 antibody. In some embodiments, the immune checkpoint inhibitor is a ligand that binds to the immune checkpoint molecule, such as PD-L1/PD-L2. In some embodiments, the immune checkpoint inhibitor is an extracellular domain of PD-1 fused to an Fc fragment of an immunoglobulin (such as IgG4 Fc). In some embodiments, the immune checkpoint inhibitor is a ligand that binds to HHLA2. In some embodiments, the immune checkpoint inhibitor is an extracellular domain of TMIGD2 fused to an Fc fragment of an immunoglobulin, such as IgG4 Fc. In some embodiments, the immune checkpoint inhibitor is a ligand that binds to at least two different inhibitory immune checkpoint molecules (e.g. bispecific), such as a ligand that binds to both CD47 and CXCR4. In some embodiments, the immune checkpoint inhibitor comprises an extracellular domain of SIRPα and a CXCL12 fragment fused to an Fc fragment of an immunoglobulin, such as IgG4 Fc.

In some embodiments, the heterologous protein is an inhibitor of an immunoinhibitory receptor. The immunoinhibitory receptor can be any receptor expressed by an immune effector cell that inhibits or reduces an immune response to tumor cells. Exemplary effector cell includes without limitation a T lymphocyte, a B lymphocyte, a natural killer (NK) cell, a dendritic cell (DC), a macrophage, a monocyte, a neutrophil, an NKT-cell, or the like. In some embodiments, the immunoinhibitory receptor is LILRB, TYRO3, AXL, or MERTK. In some embodiments, the inhibitor of an immunoinhibitory receptor is an anti-LILRB antibody.

In some embodiments, the heterologologous protein promotes an M2 to M1 switch in a macrophage population. In some embodiments, the heterologous protein is a secreted anti-LILRB antibody, wherein the antibody is an antagonist of LILRB.

In some embodiments, the heterologous protein is a multispecific immune cell engager. In some embodiments, the multispecific immune cell engager is a bispecific immune cell engager. In some embodiments, the heterologous protein is a bispecific T cell engager (BiTE). Exemplary bispecific immune cell engagers have been described, for example, in international patent publication WO2018049261, herein incorporated by reference in its entirety. In some embodiments, the bispecific immune cell engager comprises a first antigen-binding domain (such as scFv) specifically recognizing a tumor antigen (such as EpCAM, FAP, or EGFR) and a second antigen-binding domain (such as scFv) specifically recognizing a cell surface molecule on an effector cell (such as CD3 on T lymphocytes). Tumor antigens can be a tumor-associated antigen (TAA) or a tumor-specific antigen (TSA). In some embodiments, TAA or TSA is expressed on a cell of a solid tumor. Tumor antigens include, but are not limited to, EpCAM, FAP, EphA2, HER2, GD2, EGFR, VEGFR2, and Glypican-3 (GPC3). In some embodiments, the tumor antigen is EpCAM. In some embodiments, the tumor antigen is FAP. In some embodiments, the tumor antigen is EGFR.

As described above, effector cells include, but are not limited to T lymphocyte, B lymphocyte, natural killer (NK) cell, dendritic cell (DC), macrophage, monocyte, neutrophil, NKT-cell, or the like. In some embodiments, the effector cell is a T lymphocyte. In some embodiments, the effector cell is a cytotoxic T lymphocyte. Cell surface molecules on an effector cell include, but are not limited to CD3, CD4, CD5, CD8, CD16, CD28, CD40, CD64, CD89, CD134, CD137, NKp46, NKG2D, or the like. In some embodiments, the cell surface molecule is CD3.

A cell surface molecule on an effector cell of the present application is a molecule found on the external cell wall or plasma membrane of a specific cell type or a limited number of cell types. Examples of cell surface molecules include, but are not limited to, membrane proteins such as receptors, transporters, ion channels, proton pumps, and G protein-coupled receptors; extracellular matrix molecules such as adhesion molecules (e.g., integrins, cadherins, selectins, or NCAMS); see, e.g., U.S. Pat. No. 7,556,928, which is incorporated herein by reference in its entirety. Cell surface molecules on an effector cell include but not limited to CD3, CD4, CD5, CD8, CD16, CD27, CD28, CD40, CD64, CD89, CD134, CD137, CD278, NKp46, NKp30, NKG2D, and an invariant TCR.

The cell surface molecule-binding domain of an engager molecule can provide activation to immune effector cells. The skilled artisan recognizes that immune cells have different cell surface molecules. For example CD3 is a cell surface molecule on T-cells, whereas CD16, NKG2D, or NKp30 are cell surface molecules on NK cells, and CD3 or an invariant TCR are the cell surface molecules on NKT-cells. Engager molecules that activate T-cells may therefore have a different cell surface molecule-binding domain than engager molecules that activate NK cells. In some embodiments, e.g., wherein the immune cell is a T-cell, the activation molecule is one or more of CD3, e.g., CD3γ, CD3δ or CD3ε; or CD27, CD28, CD40, CD134, CD137, and CD278. In other some embodiments, e.g., wherein the immune cell is a NK cell, the cell surface molecule is CD16, NKG2D, or NKp30, or wherein the immune cell is a NKT-cell, the cell surface molecule is CD3 or an invariant TCR.

CD3 comprises three different polypeptide chains (ε, δ and γ chains), and is an antigen expressed by T cells. The three CD3 polypeptide chains associate with the T-cell receptor (TCR) and the ζ-chain to form the TCR complex, which has the function of activating signaling cascades in T cells. Currently, many therapeutic strategies target the TCR signal transduction to treat diseases using anti-human CD3 monoclonal antibodies. The CD3 specific antibody OKT3 is the first monoclonal antibody approved for human therapeutic use, and is clinically used as an immunomodulator for the treatment of allogenic transplant rejections.

In some embodiments, the heterologous protein is a cytokine. In some embodiments, the heterologous protein is IL-15, IL-12, IL-18, CXCL10, or CCL4, or a fusion protein derived therefrom. In some embodiments, the heterologous protein is a fusion protein comprising an inflammatory cytokine and a stabilizing domain. The stabilizing domain can be any suitable domain that stabilizes the inhibitory polypeptide. In some embodiments, the stabilizing domain extends the half-life of the inhibitory polypeptide in vivo. In some embodiments, the stabilizing domain is an Fc domain. In some embodiments, the stabilizing domain is an albumin domain.

In some embodiments, the Fc domain is selected from the group consisting of Fc fragments of IgG, IgA, IgD, IgE, IgM, and combinations and hybrids thereof. In some embodiments, the Fc domain is derived from a human IgG. In some embodiments, the Fc domain comprises the Fc domain of human IgG1, IgG2, IgG3, IgG4, or a combination or hybrid IgG. In some embodiments, the Fc domain has a reduced effector function as compared to corresponding wildtype Fc domain (such as at least about 30%, 40%, 50%, 60%, 70%, 80%, 85%, 90%, or 95% reduced effector function as measured by the level of antibody-dependent cellular cytotoxicity (ADCC)).

In some embodiments, the inflammatory cytokine and the stabilization domain are fused to each other via a linker, such as a peptide linker. A peptide linker may have a naturally occurring sequence, or a non-naturally occurring sequence. For example, a sequence derived from the hinge region of heavy chain only antibodies may be used as the linker. The peptide linker can be of any suitable length. In some embodiments, the peptide linker tends not to adopt a rigid three-dimensional structure, but rather provide flexibility to a polypeptide. In some embodiments, the peptide linker is a flexible linker. Exemplary flexible linkers include glycine polymers, glycine-serine polymers, glycine-alanine polymers, alanine-serine polymers, and other flexible linkers known in the art.

In some embodiments, the engineered immune cell comprises two or more additional nucleotide sequences, wherein each nucleotide sequence encodes any one of the heterologous proteins described herein.

### Antagonists or Inhibitors

Antagonist, as used herein, is interchangeable with inhibitor. In some embodiments, the heterologous protein is an inhibitor (i.e., an antagonist) of a target protein, wherein the target protein is an immunoinhibitory protein (e.g., a checkpoint inhibitor, complement regulatory protein, or other inhibitor of immune cell activation). In some embodiments, the heterologous protein is an inhibitor (i.e., an antagonist) of CD55 or CD59. In some embodiments, the target protein is an immune checkpoint protein. In some embodiments, the target protein is PD-1, PD-L1, PD-L2, CD47, CXCR4, CSF1R, LAG-3, TIM-3, HHLA2, BTLA, CD160, CD73, CTLA-4, B7-H4, TIGIT, VISTA, or 2B4. In some embodiments, the target protein is CTLA-4, PD-1, PD-L1, B7-H4, or HLA-G. In some embodiments, the target protein is an immunoinhibitory receptor selected from LILRB, TYRO3, AXL, or MERTK. In some embodiments, the target protein is LILRB. In some embodiments, inhibition of LILRB by a secreted LILRB antagonist (e.g., by a secreted anti-LILRB antibody) promotes an M2 to M1 transition in a macrophage population. In some embodiments, inhibition of LILRB with an antagonist secreted by the engineered immune cells reduces the ratio of M2 to M1 cells in a tumor microenvironment of an individual.

The antagonist inhibits the expression and/or activity of the target protein (e.g., an immunoinhibitory receptor or an immune checkpoint protein). In some embodiments, the antagonist inhibits expression of the target protein (e.g., mRNA or protein level) by at least about any one of 5%, 10%, 15%, 20%, 25%, 30%, 35%, 40%, 55%, 60%, 65%, 70%, 75%, 80%, 85%, 90%, 95% or more. Expression levels of a target protein can be determined using known methods in the art, including, for example, quantitative Polymerase Chain Reaction (qPCR), microarray, and RNA sequencing for determining RNA levels; and Western blots and enzyme-linked immunosorbent assays (ELISA) for determining protein levels.

In some embodiments, the antagonist inhibits activity (e.g., binding to a ligand or receptor of the target protein, or enzymatic activity) of the target protein by at least about any one of 5%, 10%, 15%, 20%, 25%, 30%, 35%, 40%, 55%, 60%, 65%, 70%, 75%, 80%, 85%, 90%, 95% or more. Binding can be assessed using known methods in the art, including, for example, Surface Plasmon Resonance (SPR) assays, and gel shift assays.

The antagonist may be of any suitable molecular modalities, including, but are not limited to, antibodies, inhibitory polypeptides, fusion proteins, etc.

i. Antibodies

In some embodiments, the antagonist inhibits binding of the target protein (e.g., an immune checkpoint protein or immunoinhibitory protein) to a ligand or a receptor. In some embodiments, the antagonist is an antibody that specifically binds to the target protein (e.g., CD55, CD59, CTLA-4, PD-1, PD-L1, B7-H4, HLA-G, LILRB, TYRO3, AXL, or MERTK), or an antigen-binding fragment thereof. In some embodiments, the antagonist is a polyclonal antibody. In some embodiments, the antagonist is a monoclonal antibody. In some embodiments, the antagonist is a full-length antibody, or an immunoglobulin derivative. In some embodiments, the antagonist is an antigen-binding fragment. Exemplary antigen-binding fragments include, but are not limited to, a single-chain Fv (scFv), a Fab, a Fab′, a F(ab′)₂, a Fv, a disulfide stabilized Fv fragment (dsFv), a (dsFv)₂, a single-domain antibody (e.g., VHH), a Fv-Fc fusion, a scFv-Fc fusion, a scFv-Fv fusion, a diabody, a tribody, and a tetrabody. In some embodiments, the antagonist is a scFv. In some embodiments, the antagonist is a Fab or Fab′. In some embodiments, the antagonist is a chimeric, human, partially humanized, fully humanized, or semi-synthetic antibody. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains.

In some embodiments, the antibody comprises one or more antibody constant regions, such as human antibody constant regions. In some embodiments, the heavy chain constant region is of an isotype selected from IgA, IgG, IgD, IgE, and IgM. In some embodiments, the human light chain constant region is of an isotype selected from κ and λ. In some embodiments, the antibody comprises an IgG constant region, such as a human IgG1, IgG2, IgG3, or IgG4 constant region. In some embodiments, when effector function is desirable, an antibody comprising a human IgG1 heavy chain constant region or a human IgG3 heavy chain constant region may be selected. In some embodiments, when effector function is not desirable, an antibody comprising a human IgG4 or IgG2 heavy chain constant region may be selected. In some embodiments, the antibody comprises a human IgG4 heavy chain constant region. In some embodiments, the antibody comprises an S241P mutation in the human IgG4 constant region.

In some embodiments, the antibody comprises an Fc domain. The term “Fc region,” “Fc domain” or “Fc” refers to a C-terminal non-antigen binding region of an immunoglobulin heavy chain that contains at least a portion of the constant region. The term includes native Fc regions and variant Fc regions. In some embodiments, a human IgG heavy chain Fc region extends from Cys226 to the carboxyl-terminus of the heavy chain. However, the C-terminal lysine (Lys447) of the Fc region may or may not be present, without affecting the structure or stability of the Fc region. Unless otherwise specified herein, numbering of amino acid residues in the IgG or Fc region is according to the EU numbering system for antibodies, also called the EU index, as described in Kabat et al., Sequences of Proteins of Immunological Interest, 5th Ed. Public Health Service, National Institutes of Health, Bethesda, M D, 1991. In some embodiments, the antibody comprises a variant Fc region has at least one amino acid substitution compared to the Fc region of a wild type IgG or a wild-type antibody.

In some embodiments, the antibody is altered to increase or decrease the extent to which the antibody is glycosylated. Addition or deletion of glycosylation sites to an antibody may be conveniently accomplished by altering the amino acid sequence such that one or more glycosylation sites is created or removed.

Antibodies that specifically bind to a target protein can be obtained using methods known in the art, such as by immunizing a non-human mammal and obtaining hybridomas therefrom, or by cloning a library of antibodies using molecular biology techniques known in the art and subsequence selection or by using phage display.

### III. Methods of Treatment

The present application provides methods of treating a cancer (e.g., solid tumor or liquid tumor) in an individual in need thereof, comprising administering to the individual an effective amount of any one of the engineered immune cells comprising a heterologous heterologous nucleotide sequence encoding a sialidase or compositions (e.g., pharmaceutical compositions) described herein. In some embodiments, there is provided a method of treating a cancer in an individual in need thereof, comprising administering to the individual an effective amount of an engineered immune cell comprising a first heterologous nucleotide sequence encoding a sialidase and a second heterologous nucleotide sequence encoding a chimeric immune receptor. In some embodiments, the sialidase is a bacterial sialidase (e.g., a Clostridium perfringens sialidase, Actinomyces viscosus sialidase, and Arthrobacter ureafaciens sialidase, Salmonella typhimurium sialidase or Vibrio cholera sialidase) or a derivative thereof. In some embodiments, the sialidase is derived from a Actinomyces viscosus sialidase. In some embodiments, the sialidase is DAS181. the heterologous nucleotide sequence encoding the sialidase further encodes a secretion sequence (e.g., a signal sequence or signal peptide) operably linked to the sialidase. In some embodiments, the sialidase further comprises a transmembrane domain. In some embodiments, the chimeric receptor specifically recognizes a tumor associated antigen.

In some embodiments, there is provided a method of treating a cancer in an individual in need thereof, comprising administering to the individual: (a) an effective amount of a first engineered immune cell comprising a first heterologous nucleotide sequence encoding a sialidase; and (b) an effective amount of a second engineered immune cell expressing a chimeric receptor. In some embodiments, the sialidase is a bacterial sialidase (e.g., Clostridium perfringens sialidase, Actinomyces viscosus sialidase, and Arthrobacter ureafaciens sialidase, Salmonella typhimurium sialidase or Vibrio cholera sialidase). In some embodiments, the sialidase comprises an anchoring domain. In some embodiments, the anchoring domain is a GAG-binding protein domain, e.g., the epithelial anchoring domain of human amphiregulin. In some embodiments, the anchoring domain is positively charged at physiologic pH. In some embodiments, the anchoring domain is a GPI linker. In some embodiments, the sialidase is DAS181. In some embodiments, the sialidase comprises a transmembrane domain. In some embodiments, the chimeric receptor recognizes a tumor-associated antigen or tumor-specific antigen. In some embodiments, the engineered immune cells are T cells or NK cells. In some embodiments, the chimeric receptor is a CAR. In some embodiments, the chimeric immune receptor specifically recognizes a tumor antigen, such as, without limitation, carcinoembryonic antigen, alphafetoprotein, MUC16, survivin, glypican-3, B7 family members, VISTA, MICA/B, LILRB, CD19, BCMA, NY-ESO-1, CD20, CD22, CD24, CD33, CD38, CD200, CEA, EGFRvIII, Integrin beta 1, Integrin beta 4, GD2, HER2, IGF1R, mesothelin, PSMA, ROR1, WT1, NY-ESO-1, and CDH17, or other tumor antigens with clinical significance. In some embodiments, the chimeric immune cell receptor is an anti-CD19 CAR In some embodiments, the chimeric immune receptor is an anti-CDH17 CAR cell. In some embodiments, the first and second engineered immune cell are administered in a 1:5, 1:4, 1:3, 1:2, 1.5:1, 1:1, 1:1.5, 2:1, 3:1, 4:1, or 5:1 ratio. In some embodiments, the first engineered immune cell and the second engineered immune cell are present in the composition in a 1:1 ratio. In some embodiments, the first and second engineered immune cells are administered simultaneously (e.g., in a single composition). In some embodiments, the first and second engineered immune cells are administered in separate formulations. In some embodiments, the first and second engineered immune cells are administered sequentially. In some embodiments, the first engineered immune cell is administered before the second engineered immune cell. In some embodiments, the second engineered immune cell is administered before the first engineered immune cell.

In some embodiments, there is provided a method of treating a cancer in an individual in need thereof, comprising administering to the individual an effective amount of an engineered immune cell comprising a first heterologous nucleotide sequence encoding a bacterial sialidase and a second heterologous nucleotide sequence encoding a chimeric immune receptor, wherein the bacterial sialidase is a secreted membrane-associated protein or a membrane-bound protein. In some embodiments, the secreted sialidase comprises an anchoring domain. In some embodiments, the anchoring domain limits diffusion of the sialidase. In some embodiments, the sialidase reduces sialylation of cancer cells and/or immune cells in a tumor microenvironment. In some embodiments, the sialidase reduces surface sialic acid on tumor cells and/or immune cells by at least 10%, 15%, 20%, 30%, 40%, 50%, 60%, 70%, 75%, 80%, 85%, or 90%, and does not substantially reduce surface sialic acid on other cells in the individual. In some embodiments, there is provided a method of treating a cancer in an individual in need thereof, comprising administering to the individual an effective amount of an engineered immune cell comprising a first heterologous nucleotide sequence encoding a bacterial sialidase and a second heterologous nucleotide sequence encoding a chimeric immune receptor. In some embodiments, the sialidase is Clostridium perfringens sialidase, Actinomyces viscosus sialidase, and Arthrobacter ureafaciens sialidase, Salmonella typhimurium sialidase or Vibrio cholera sialidase. In some embodiments, the sialidase comprises an anchoring domain. In some embodiments, the anchoring domain is a GAG-binding protein domain, e.g., the epithelial anchoring domain of human amphiregulin. In some embodiments, the anchoring domain is positively charged at physiologic pH. In some embodiments, the anchoring domain is a GPI linker. In some embodiments, the sialidase is DAS181. In some embodiments, the sialidase comprises a transmembrane domain. In some embodiments, the chimeric receptor specifically recognizes the sialidase (e.g., DAS181) and is not cross-reactive with human native amphiregulin or any other human antigen. In some embodiments, the engineered immune cells are T cells or NK cells. In some embodiments, the chimeric receptor is a CAR. In some embodiments, the chimeric immune receptor specifically recognizes a tumor antigen, such as, without limitation, carcinoembryonic antigen, alphafetoprotein, MUC16, survivin, glypican-3, B7 family members, VISTA, MICA/B, LILRB, CD19, BCMA, NY-ESO-1, CD20, CD22, CD24, CD33, CD38, CD200, CEA, EGFRvIII, Integrin beta 1, Integrin beta 4, GD2, HER2, IGF1R, mesothelin, PSMA, ROR1, WT1, NY-ESO-1, and CDH17, and other tumor antigens with clinical significance. In some embodiments, the chimeric immune cell receptor is an anti-CD19 CAR. In some embodiments, the chimeric immune receptor is an anti-CDH17 CAR cell.

In some embodiments, there is provided a method of reducing sialylation of cancer cells and/or immune cells in an individual, comprising administering to the individual an effective amount of any one of the engineered immune cell compositions or pharmaceutical compositions described herein. In some embodiments, the composition comprises an engineered immune cell (e.g., a T cell, NK cell, or NKT cell) comprising a first heterologous nucleotide sequence encoding a sialidase (e.g., an Actinomyces viscosus sialidase or a derivative thereof, such as DAS181) and a second heterologous nucleotide sequence encoding a chimeric immune receptor (e.g., a CAR). In some embodiments, the composition comprises a first engineered immune cell (e.g., a T cell, NK cell, or NKT cell) comprising a first heterologous nucleotide sequence encoding a sialidase (e.g., an Actinomyces viscosus sialidase or a derivative thereof, such as DAS181), and a second engineered immune cell comprising a second heterologous nucleotide sequence encoding a chimeric immune receptor (e.g., a CAR). In some embodiments the sialidase reduces surface sialic acid on tumor cells and/or immune cells by at least 10%, 15%, 20%, 30%, 40%, 50%, 60%, 70%, 75%, 80%, 85%, or 90%. In some embodiments, the immune cells are immune cells in the tumor microenvironment.

In some embodiments, there is provided a method of reducing sialylation of cancer cells in an individual, comprising administering to the individual an effective amount of any one of the engineered immune cell compositions or pharmaceutical compositions described herein. In some embodiments, the composition comprises an engineered immune cell (e.g., a T cell, NK cell, or NKT cell) comprising a first heterologous nucleotide sequence encoding a sialidase (e.g., an Actinomyces viscosus sialidase or a derivative thereof, such as DAS181) and a second heterologous nucleotide sequence encoding a chimeric immune receptor (e.g., a CAR). In some embodiments, the composition comprises a first engineered immune cell (e.g., a T cell, NK cell, or NKT cell) comprising a first heterologous nucleotide sequence encoding a sialidase (e.g., an Actinomyces viscosus sialidase or a derivative thereof, such as DAS181), and a second engineered immune cell comprising a second heterologous nucleotide sequence encoding a chimeric immune receptor (e.g., a CAR). In some embodiments, the chimeric immune receptor specifically recognizes a tumor antigen, such as, without limitation, carcinoembryonic antigen, alphafetoprotein, MUC16, survivin, glypican-3, B7 family members, VISTA, MICA/B, LILRB, CD19, BCMA, NY-ESO-1, CD20, CD22, CD24, CD33, CD38, CD200, CEA, EGFRvIII, Integrin beta 1, Integrin beta 4, GD2, HER2, IGF1R, mesothelin, PSMA, ROR1, WT1, NY-ESO-1, and CDH17, and other tumor antigens with clinical significance. In some embodiments, the chimeric immune cell is an anti-CD19 CAR. In some embodiments the sialidase reduces surface sialic acid on tumor cells by at least 10%, 15%, 20%, 30%, 40%, 50%, 60%, 70%, 75%, 80%, 85%, or 90%.

In some embodiments, there is provided a method of reducing sialylation of immune cells in an individual, comprising administering to the individual an effective amount of any one of the engineered immune cell compositions or pharmaceutical compositions described herein. In some embodiments, the composition comprises an engineered immune cell (e.g., a T cell, NK cell, or NKT cell) comprising a first heterologous nucleotide sequence encoding a sialidase (e.g., an Actinomyces viscosus sialidase or a derivative thereof, such as DAS181) and a second heterologous nucleotide sequence encoding a chimeric immune receptor (e.g., a CAR). In some embodiments, the composition comprises a first engineered immune cell (e.g., a T cell, NK cell, or NKT cell) comprising a first heterologous nucleotide sequence encoding a sialidase (e.g., an Actinomyces viscosus sialidase or a derivative thereof, such as DAS181), and a second engineered immune cell comprising a second heterologous nucleotide sequence encoding a chimeric immune receptor (e.g., a CAR). In some embodiments, the method comprises reducing sialylation of immune cells in a tumor microenvironment. In some embodiments the sialidase reduces surface sialic acid on immune cells in the tumor microenvironment by at least 10%, 15%, 20%, 30%, 40%, 50%, 60%, 70%, 75%, 80%, 85%, or 90%. In some embodiments, reducing sialylation of immune cells in the tumor microenvironment contributes to regulation of the inflammatory response in the tumor microenvironment. For example, see Nan, X., I. Carubelli, and N. M. Stamatos, Sialidase expression in activated human T lymphocytes influences production of IFN-gamma. J Leukoc Biol, 2007. 81(1): p. 284-96; Seyrantepe, V., et al., Regulation of phagocytosis in macrophages by neuraminidase 1. J Biol Chem, 2010. 285(1): p. 206-15; and Amith, S. R., et al., Neu1 desialylation of sialyl alpha-2.3-linked beta-galactosyl residues of TOLL-like receptor 4 is essential for receptor activation and cellular signaling. Cell Signal, 2010. 22(2): p. 314-24; each of which is herein incorporated by reference in its entirety.

In some embodiments, there is provided a method of inhibiting tumor growth in an individual in need thereof, comprising administering to the individual an effective amount of any one of the engineered immune cell compositions or pharmaceutical compositions described herein. In some embodiments, the composition comprises an engineered immune cell (e.g., a T cell, NK cell, or NKT cell) comprising a first heterologous nucleotide sequence encoding a sialidase (e.g., an Actinomyces viscosus sialidase or a derivative thereof, such as DAS181) and a second heterologous nucleotide sequence encoding a chimeric immune receptor (e.g., a CAR). In some embodiments, the composition comprises a first engineered immune cell (e.g., a T cell, NK cell, or NKT cell) comprising a first heterologous nucleotide sequence encoding a sialidase (e.g., an Actinomyces viscosus sialidase or a derivative thereof, such as DAS181), and a second engineered immune cell comprising a second heterologous nucleotide sequence encoding a chimeric immune receptor (e.g., a CAR). In some embodiments, the sialidase is in secreted or membrane-bound form. In some embodiments, the engineered immune encoding a sialidase and a chimeric immune receptor reduces tumor growth by at least 1.5, 2, 2.5, 3, 4, 5, 10, 20, 30, or 40 fold. In some embodiments, the engineered immune cell encoding a sialidase increases the inhibition of tumor growth by CAR-NK cells by at least 1.5, 2, 2.5, 3, 4, 5, 8, or 10 fold compared to NK cells lacking a sialidase. In some embodiments, the engineered immune cell encoding a sialidase increases inhibition of tumor growth by CAR-NK cells by at least 1.5, 2, 2.5, 3, 4, 5, 8, or 10 fold compared to NK cells encoding a Neu2 sialidase. In some embodiments, the engineered immune cell encoding a sialidase increases inhibition of tumor growth by CAR-T cells by at least 1.5, 2, 2.5, 3, 4, 5, 10, 20, 30, or 40 fold. In some embodiments, the engineered immune cell encoding a sialidase increases inhibition of tumor growth by CAR-T cells by at least 1.5, 2, 2.5, 3, 4, 5, 8, or 10 fold compared to T cells lacking sialidase. In some embodiments, the engineered immune cell encoding a sialidase increases inhibition of tumor growth by CAR-T cells by at least 1.5, 2, 2.5, 3, 4, 5, 8, or 10 fold compared to T cells encoding a Neu2 sialidase.

In some embodiments, there is provided a method of killing cancer cells in an individual in need thereof, comprising administering to the individual an effective amount of any one of the engineered immune cell compositions or pharmaceutical compositions described herein. In some embodiments, the composition comprises an engineered immune cell (e.g., a T cell, NK cell, or NKT cell) comprising a first heterologous nucleotide sequence encoding a sialidase (e.g., an Actinomyces viscosus sialidase or a derivative thereof, such as DAS181) and a second heterologous nucleotide sequence encoding a chimeric immune receptor (e.g., a CAR). In some embodiments, the composition comprises a first engineered immune cell (e.g., a T cell, NK cell, or NKT cell) comprising a first heterologous nucleotide sequence encoding a sialidase (e.g., an Actinomyces viscosus sialidase or a derivative thereof, such as DAS181), and a second engineered immune cell comprising a second heterologous nucleotide sequence encoding a chimeric immune receptor (e.g., a CAR). In some embodiments, the sialidase cleaves both α2,3 and α2,6 sialic acids from the cell surface of tumor cells. In some embodiments, the sialidase increases cleavage of both α2,3 and α2,6 sialic acids by at least 10%, 15%, 20%, 30%, 40%, 50%, 60%, 70%, 75%, 80%, 85%, or 90%.

In some embodiments, there is provided a method of treating a cancer in an individual in need thereof, comprising administering an effective amount of an engineered immune cell comprising a heterologous nucleotide sequence encoding DAS181, wherein the engineered immune cell is a T-cell, a natural killer (NK) cell, a macrophage, or a natural killer T (NKT) cell, and wherein the DAS181 reduces sialylation on the surface of tumor cells. In some embodiments, the heterologous nucleotide sequence further encodes a secretion sequence operably linked to the DAS181. In some embodiments, the DAS181 comprises an anchoring domain. In some embodiments, the DAS181 comprises a transmembrane domain.

In some embodiments, there is provided a method of treating cancer in an individual in need thereof, comprising administering an effective amount of an engineered immune cell comprising a heterologous nucleotide sequence encoding a sialidase, wherein the sialidase comprises a sequence having at least about 80% (e.g., at least 81%, 82%, 83%, 84%, 85%, 86%, 87%, 88%, or 89%) at least about 90% (e.g., at least 90%, 91%, 92%, 93%, 94%, 95%, 96%, 97%, 98%, 99%, or 100%) sequence identity to the amino acid sequence of SEQ ID NO: 1, and wherein the engineered immune cell is a T-cell, a natural killer (NK) cell, a macrophage, or a natural killer T (NKT) cell. In some embodiments, the heterologous nucleotide sequence further encodes a secretion sequence operably linked to the In some embodiments, the sialidase is a fusion protein comprising a sialidase catalytic domain fused to an anchoring domain. In some embodiments, the sialidase comprises a transmembrane domain. In some embodiments, the sialidase cleaves both α-2, 3 and α-2, 6 linkages on the surface of tumor cells.

In some embodiments, there is provided a method of sensitizing a tumor in an individual to an immunotherapy, comprising administering to the individual an effective amount of any one of the engineered immune cells comprising a heterologous nucleotide sequence encoding a sialidase described above. In some embodiments, the sialidase is a bacterial sialidase (e.g., a Clostridium perfringens sialidase, Actinomyces viscosus sialidase, and Arthrobacter ureafaciens sialidase, Salmonella typhimurium sialidase or Vibrio cholera sialidase) or a derivative thereof. In some embodiments, the sialidase is derived from a Actinomyces viscosus sialidase. In some embodiments, the sialidase is DAS181. In some embodiments, the heterologous nucleotide sequence encoding the sialidase further encodes a secretion sequence (e.g., a signal sequence or signal peptide) operably linked to the sialidase. In some embodiments, the sialidase further comprises a transmembrane domain. In some embodiments, the method further comprises administering an effective amount of the immunotherapy to the individual. In some embodiments, the immunotherapy is selected from the group consisting of a multispecific immune cell engager (e.g., a BiTE), a cell therapy, a cancer vaccine (e.g., a dendritic cell (DC) cancer vaccine), a cytokine (e.g., IL-15, IL-12, CXCL10, or CCL4), an inhibitor of a complement regulatory protein (e.g., an inhibitor of CD55 or CD59), and an immune checkpoint inhibitor (e.g., an inhibitor of CTLA-4, PD-1, PD-L1, B7-H4, or HLA-G).

In some embodiments, the immunotherapy is a cell therapy. A cell therapy comprises administering an effective amount of live cells (e.g., immune cells) to the individual. In non-limiting examples, the immune cells can be T-cells, natural killer (NK) cells, natural killer T (NKT) cells, dendritic cells (DC), cytokine-induced killer (CIK) cells, cytokine-induced natural killer (CINK) cells, lymphokine-activated killer (LAK) cells, tumor-infiltrating lymphocytes (TILs), macrophages, or combinations thereof. In some embodiments, the cell therapy can comprise administering a developmental intermediate (e.g., a progenitor) of any one of the immune cell types described herein. In some embodiments, the cell therapy agents can comprise indiscrete heterogeneous cell populations, such as expanded PBMCs that have proliferated and acquired killing activity on ex vivo culture. Suitable cell therapies have been described, for example, in Hayes, C. “Cellular immunotherapies for cancer.” Ir J Med Sci (2020). In some embodiments, the cell therapy comprises PBMC cells that have been stimulated with various cytokine and antibody combinations to activate effector T cells (CD3, CD38 and IL-2) or, in some cases, T cells and NK cells (CD3, CD28, IL-15 and IL-21).

In some embodiments, the cell therapy comprises administering to the individual an effective amount of immune cells, wherein the immune cells have been primed to respond to a tumor antigen, e.g, by exposure to the antigen either in vivo or ex vivo.

In some embodiments, the method further comprises administering an additional immunotherapy. In some embodiments, the additional immunotherapy is a multispecific immune cell engager (e.g., a BiTE), a cell therapy, a cancer vaccine (e.g., a dendritic cell (DC) cancer vaccine), a cytokine (e.g., IL-15, IL-12, IL-18, CXCL10, or CCL4), an inhibitor of a complement regulatory protein (e.g., an inhibitor of CD55 or CD59), and an immune checkpoint inhibitor (e.g., an inhibitor of CTLA-4, PD-1, PD-L1, B7-H4, or HLA-G). In some embodiments, the immunotherapy is cell therapy, e.g., a cell therapy comprising T-cells, natural killer (NK) cells, natural killer T (NKT) cells, dendritic cells (DC), cytokine-induced killer (CIK) cells, cytokine-induced natural killer (CINK) cells, lymphokine-activated killer (LAK) cells, tumor-infiltrating lymphocytes (TILs), macrophages, or combinations thereof. In some embodiments, any one of the engineered immune cells described herein is administered before, after, or simultaneously with the immunotherapy. In some embodiments, administering the engineered immune cell increases tumor cell killing by at least 10%, 15%, 20%, 25%, 30%, 35%, 40%, 50%, 60%, 70%, 80%, 85%, 90%, 95%, or 100% compared to the additional immunotherapy alone.

One aspect of the present application provides methods of reducing sialylation of cancer cells in an individual, comprising administering to the individual an effective amount of any one of the engineered immune cells comprising a heterologous nucleotide sequence encoding a sialidase or pharmaceutical compositions described herein. In some embodiments, the sialidase reduces surface sialic acid on tumor cells. In some embodiments the sialidase reduces surface sialic acid on tumor cells by at least 10%, 15%, 20%, 30%, 40%, 50%, 60%, 70%, 75%, 80%, 85%, or 90%. In some embodiments, the sialidase cleaves both α2,3 and α2,6 sialic acids from the cell surface of tumor cells. In some embodiments, the sialidase increases cleavage of both α2,3 and α2,6 sialic acids by at least 10%, 15%, 20%, 30%, 40%, 50%, 60%, 70%, 75%, 80%, 85%, or 90%. In some embodiments, the sialidase reduces surface sialic acid on tumor cells by at least 10%, 15%, 20%, 30%, 40%, 50%, 60%, 70%, 75%, 80%, 85%, or 90%. In some embodiments, the sialidase cleaves both α2,3 and α2,6 sialic acids from the cell surface of tumor cells. In some embodiments, the sialidase increases cleavage of both α2,3 and α2,6 sialic acids by at least 1.5, 2, 2.5, 3, 4, 5, 10, 20, 30, 40, 50, or 100 fold more than a Neu2 sialidase. In some embodiments, the sialidase reduces surface sialic acid on tumor cells by at least 1.5, 2, 2.5, 3, 4, 5, 10, 20, 30, 40, 50, or 100 fold more than a Neu2 sialidase. In some embodiments, the sialidase is an Actinomyces viscosus sialidase or a derivative thereof. In some embodiments, the sialidase is DAS181. Example 2 provides unexpected results demonstrating enhanced sialic acid removal activity of a secreted or transmembrane form of DAS181 compared to a secreted or transmembrane form of Neu2 expressed in tumor cells.

In some embodiments, there is provided a method of promoting an immune response in an individual, comprising administering to the individual an effective amount of any one of the engineered immune cells comprising a heterologous nucleotide sequence encoding a sialidase or pharmaceutical compositions described herein. In some embodiments, the method promotes a local immune response in a tumor microenvironment of the individual. In some embodiments, there is provided a method of promoting dendritic cell (DC) maturation in an individual, comprising administering an effective amount of an engineered immune cell (e.g., a CAR-T or CAR-NK cell) encoding a sialidase (e.g., DAS181). DC maturation can be determined based on the expression of dendritic cell markers, such as CD80 and DC MHC I and MHC-II proteins. In some embodiments, the engineered immune cell encoding a sialidase increases DC maturation by at least 1.5, 2, 2.5, 3, 4, 5, or 10 fold.

In some embodiments, there is provided a method of increasing immune cell killing of tumor cells in an individual, comprising administering an effective amount of an engineered immune cell (e.g., a T cell, NK cell, or NKT cell) encoding a sialidase. In some embodiments, the sialidase is DAS181. In some embodiments, the sialidase is in secreted or membrane-bound form. In some embodiments, the method increases killing by CAR-NK cells. In some embodiments, the engineered immune encoding a sialidase increases killing by CAR-NK cells by at least 1.5, 2, 2.5, 3, 4, 5, 10, 20, 30, or 40 fold. In some embodiments, the engineered immune cell encoding a sialidase increases killing by CAR-NK cells by at least 1.5, 2, 2.5, 3, 4, 5, 8, or 10 fold compared to NK cells lacking a sialidase. In some embodiments, the engineered immune cell encoding a sialidase increases killing by CAR-NK cells by at least 1.5, 2, 2.5, 3, 4, 5, 8, or 10 fold compared to NK cells encoding a Neu2 sialidase. Example 4 demonstrates enhanced CAR-NK cell-mediated killing of tumor cells with administration of an engineered immune encoding a sialidase. In some embodiments, the method increases killing by CAR-T cells. In some embodiments, the engineered immune cell encoding a sialidase increases killing by CAR-T cells by at least 1.5, 2, 2.5, 3, 4, 5, 10, 20, 30, or 40 fold. In some embodiments, the engineered immune cell encoding a sialidase increases killing by CAR-T cells by at least 1.5, 2, 2.5, 3, 4, 5, 8, or 10 fold compared to T cells lacking sialidase. In some embodiments, the engineered immune cell encoding a sialidase increases killing by CAR-T cells by at least 1.5, 2, 2.5, 3, 4, 5, 8, or 10 fold compared to T cells encoding a Neu2 sialidase. Example 5 demonstrates enhanced T cell-mediated killing of tumor cells with administration of an engineered immune encoding a sialidase increases. In some embodiments, the method increases killing by immune cells such T-cells, natural killer (NK) cells, natural killer T (NKT) cells, dendritic cells (DC), cytokine-induced killer (CIK) cells, cytokine-induced natural killer (CINK) cells, lymphokine-activated killer (LAK) cells, tumor-infiltrating lymphocytes (TILs), macrophages, or combinations thereof. In some embodiments, administering the engineered immune cell encoding the sialidase increases killing by immune cells such T-cells, natural killer (NK) cells, natural killer T (NKT) cells, dendritic cells (DC), cytokine-induced killer (CIK) cells, cytokine-induced natural killer (CINK) cells, lymphokine-activated killer (LAK) cells, tumor-infiltrating lymphocytes (TILs), macrophages, or combinations thereof by at least 1.5, 2, 2.5, 3, 4, 5, 10, 20, 30, or 40 fold.

As used herein, cancer is a term for diseases caused by or characterized by any type of malignant tumor or hematological malignancy, including metastatic cancers, lymphatic tumors, and blood cancers In some embodiments, the cancer is a liquid tumor (e.g., lymphoma or blood cancers). In some embodiments, the cancer is lymphoma.

In some embodiments, the cancer comprises a solid tumor. In some embodiments of any of the methods provided herein, the cancer is an adenocarcinoma, a metastatic cancer and/or is a refractory cancerIn some embodiments, the cancer is a human alveolar basal epithelial adenocarcinoma, human mamillary epithelial adenocarcinoma, or glioblastoma.

In some embodiments, delivery of the sialidase via engineered immune cells can reduce sialic acid present on tumor cells and render the tumor cells more vulnerable to killing by immune cells, immune cell-based therapies and other therapeutic agents whose effectiveness is diminished by hypersialylation of cancer cells.

In some embodiments, there is provided a method of increasing immune cell infiltration of a tumor, comprising administering an effective amount of any one of the engineered immune cells expressing a sialidase described herein. In some embodiments, the engineered immune cells expressing a sialidase increase infiltration of a tumor microenvironment by engineered immune cells (e.g., CAR-T or CAR-NK cells). In some embodiments, the engineered immune cells expressing a sialidase increase infiltration of a tumor microenvironment by inflammation-promoting immune cells such as T-cells, natural killer (NK) cells, natural killer T (NKT) cells, dendritic cells (DC), cytokine-induced killer (CIK) cells, cytokine-induced natural killer (CINK) cells, lymphokine-activated killer (LAK) cells, tumor-infiltrating lymphocytes (TILs), macrophages, or combinations thereof.

In some embodiments, the engineered immune cells expressing a sialidase increase the number of M1-type macrophages in the tumor microenvironment. In some embodiments, the engineered immune cells increase the ratio of M1-type macrophages to M2-type macrophages in the tumor microenvironment In some embodiments, the engineered immune cells comprise a third heterologous nucleotide sequence encoding a heterologous protein, wherein the heterologous protein increases the ratio of M1-type to M2-type macrophages in the tumor microenvironment. In some embodiments, the engineered immune cells comprise a third heterologous nucleotide sequence encoding a secreted LILRB antagonist. In some embodiments, the secreted LILRB antagonist is an anti-LILRB antibody.

In some embodiments, the method further comprises administering to the individual an effective amount of an immunotherapeutic agent. In non-limiting examples, the immunotherapeutic agent can be a multispecific immune cell engager, a cell therapy, a cancer vaccine, a cytokine, an inhibitor of a complement regulatory protein, or an immune checkpoint inhibitor.

The engineered immune cells described herein, and optionally the additional immunotherapeutic agent(s), may be administered using any suitable routes of administration and suitable dosages. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

In some embodiments, the engineered immune cells, and optionally the additional immunotherapeutic agent(s) are administered sequentially. In some embodiments, the engineered immune cells and optional additional immunotherapeutic agent(s) are administered simultaneously or concurrently. In some embodiments, the engineered immune cells and, optionally, the additional immunotherapeutic agent(s) are administered in a single formulation. In some embodiments, the engineered immune cells and the optional additional immunotherapeutic agent(s) are administered as separate formulations.

The methods of the present invention may be combined with conventional chemotherapeutic, radiologic and/or surgical methods of cancer treatment.

### IV. Pharmaceutical Compositions, Kits and Articles of Manufacture

Further provided by the present application are pharmaceutical compositions comprising any one of the engineered immune cells encoding a sialidase described herein, and a pharmaceutically acceptable carrier. Pharmaceutical compositions can be prepared by mixing the therapeutic agents described herein having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers (e.g. sodium chloride), stabilizers, metal complexes (e.g. Zn-protein complexes); chelating agents such as EDTA and/or non-ionic surfactants.

The formulation can include a carrier. The carrier is a macromolecule which is soluble in the circulatory system and which is physiologically acceptable where physiological acceptance means that those of skill in the art would accept injection of said carrier into a patient as part of a therapeutic regime. The carrier preferably is relatively stable in the circulatory system with an acceptable plasma half-life for clearance. Such macromolecules include but are not limited to soy lecithin, oleic acid and sorbitan trioleate.

The formulations can also include other agents useful for pH maintenance, solution stabilization, or for the regulation of osmotic pressure. Examples of the agents include but are not limited to salts, such as sodium chloride, or potassium chloride, and carbohydrates, such as glucose, galactose or mannose, and the like.

In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container. In some embodiments, the pharmaceutical composition is cryopreserved.

In some embodiments, the systems provided herein can be stably and indefinitely stored under cryopreservation conditions, such as, for example, at −80° C., and can be thawed as needed or desired prior to administration. For example, the systems provided herein can be stored at a preserving temperature, such as −20° C. or −80° C., for at least or between about a few hours. 1, 2, 3, 4 or 5 hours, or days, including at least or between about a few years, such as, but not limited to, 1, 2, 3 or more years, for example for at least or about 1, 2, 3, 4 or 5 hours to at least or about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71 or 72 hours or 4, 5, 6, 7, 8, 9, 10, 15, 20, 25 or 30 days or 1.5, 2, 2.5, 3, 3.5, 4, 4.5, 5, 5.5, 6, 6.5, 7, 7.5, 8, 8.5, 9, 9.5, 10, 10.5, 11, 11.5 or 12 months or 1, 2, 3, 4 or 5 or more years prior to thawing for administration. The systems provided herein also stably can be stored under refrigeration conditions such as, at 4° C. and/or transported on ice to the site of administration for treatment. For example, the systems provided herein can be stored at 4° C. or on ice for at least or between about a few hours, such as, but not limited to, 1, 2, 3, 4 or 5 hours, to at least or about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 or more hours prior to administration for treatment.

The present application further provides kits and articles of manufacture for use in any embodiment of the treatment methods described herein. The kits and articles of manufacture may comprise any one of the formulations and pharmaceutical compositions described herein.

In some embodiments, there is provided a kit comprising one or more nucleic acid constructs for expression any one of the sialidases described herein, and instructions for producing the engineered immune cell. In some embodiments, the kit further comprises instructions for treating a cancer.

In some embodiments, there is provided a kit comprising any one of the engineered immune cells encoding a sialidase, and instructions for treating a cancer. In some embodiments, the kit further comprises one or more additional immunotherapeutic agents (e.g., a cell therapy or any one of the immunotherapies described herein). In some embodiments, the kit further comprises one or more additional therapeutic agents for treating the cancer. In some embodiments, the engineered immune cells and optionally the additional immunotherapeutic agent(s) and/or the additional therapeutic agent(s) for treating the cancer are in separate compositions. In some embodiments, there is provided a kit comprising (a) a lentiviral vector comprising a first heterologous nucleotide sequence encoding a sialidase, (b) a lentiviral vector comprising a second heterologous nucleotide sequence encoding a sialidase, and (c) instructions for preparing the engineered immune cells. In some embodiments, there is provided a single lentiviral vector comprising the first heterologous nucleotide sequence and the second lentiviral sequence.

The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

### EXAMPLES

### Example 1—Broad Activity and Potency of DAS181

This example provides results demonstrating the unexpectedly high potency and broad activity of a sialidase derived from an Actinomyces viscosus sialidase (DAS181), wherein the sialidase comprises an anchoring domain.

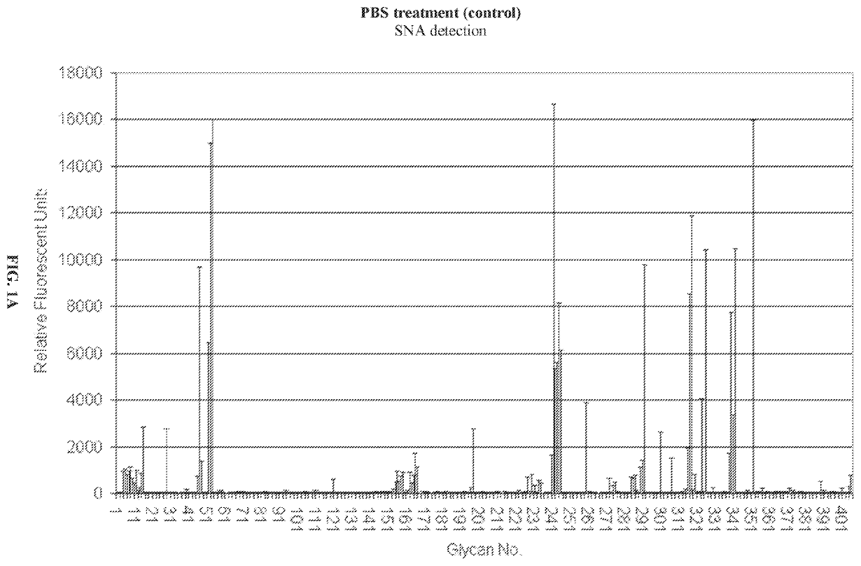

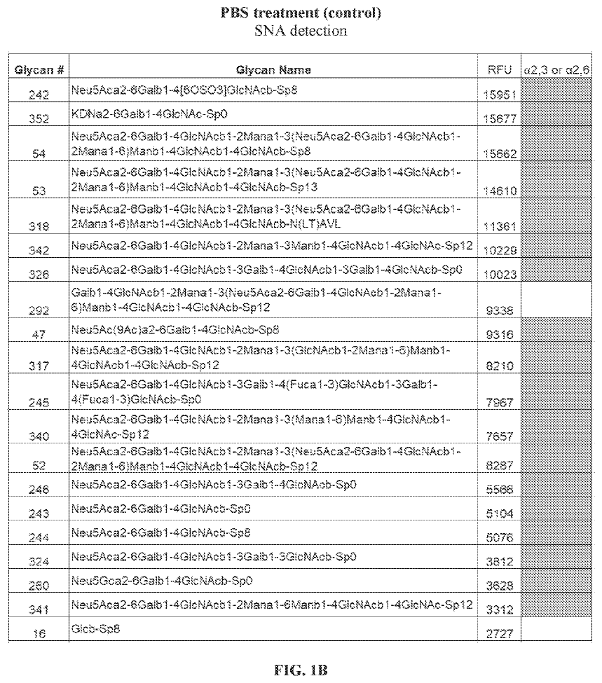

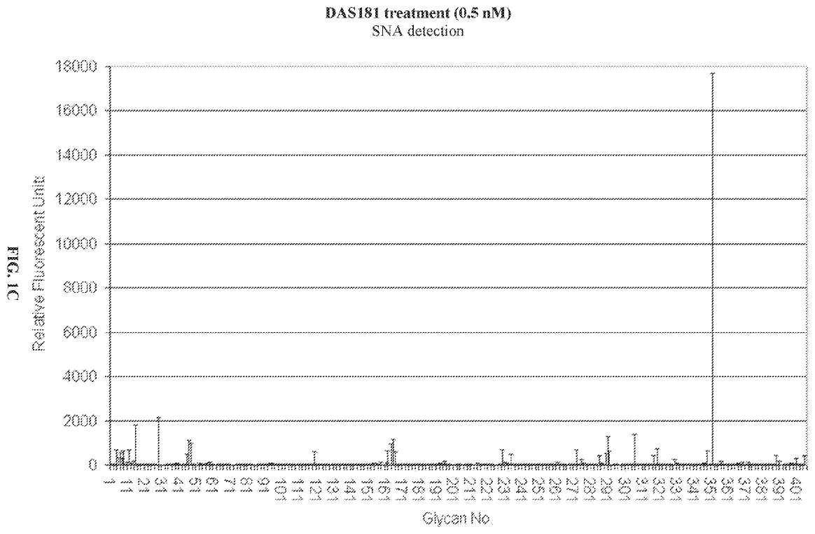

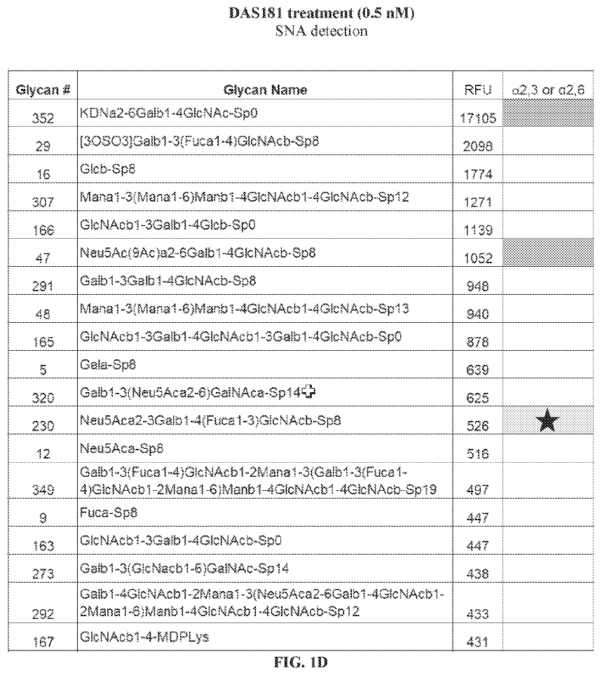

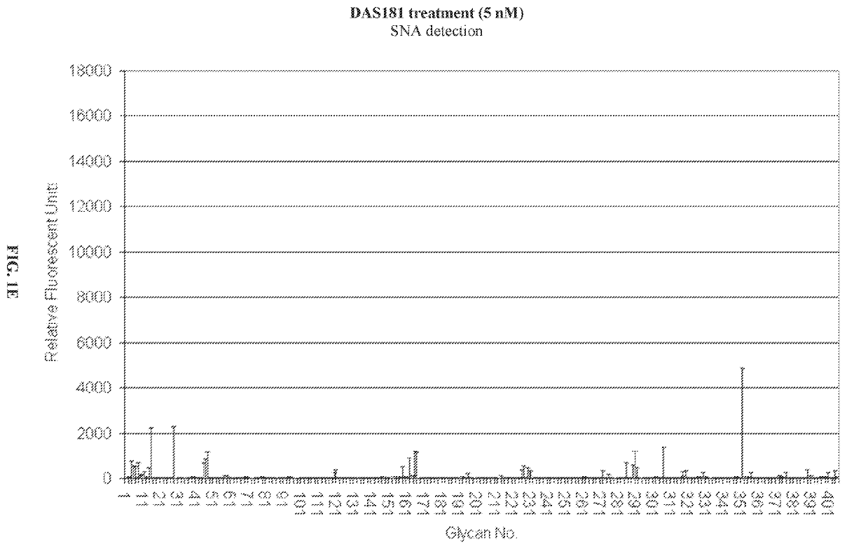

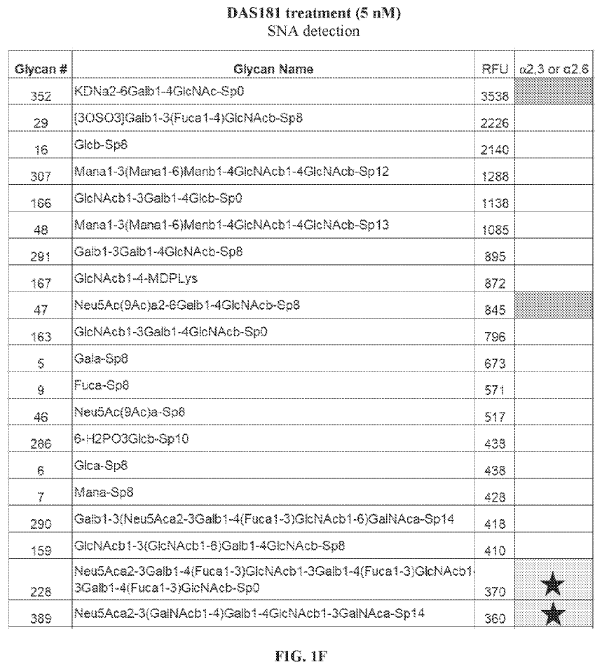

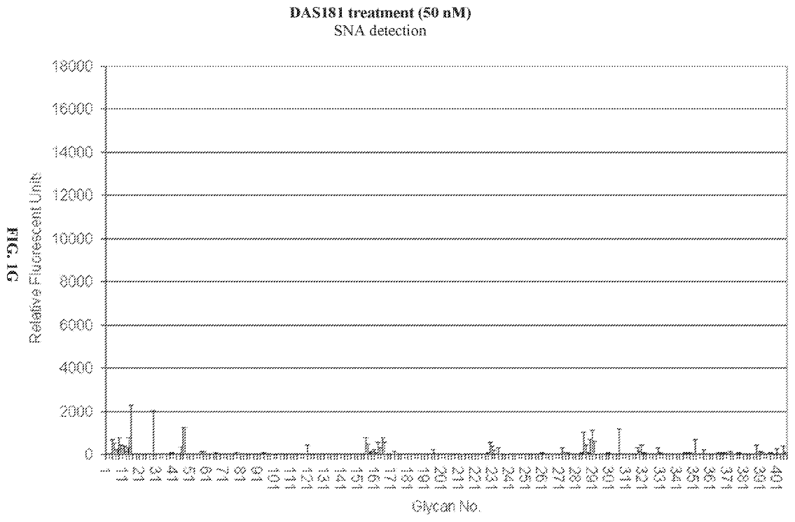

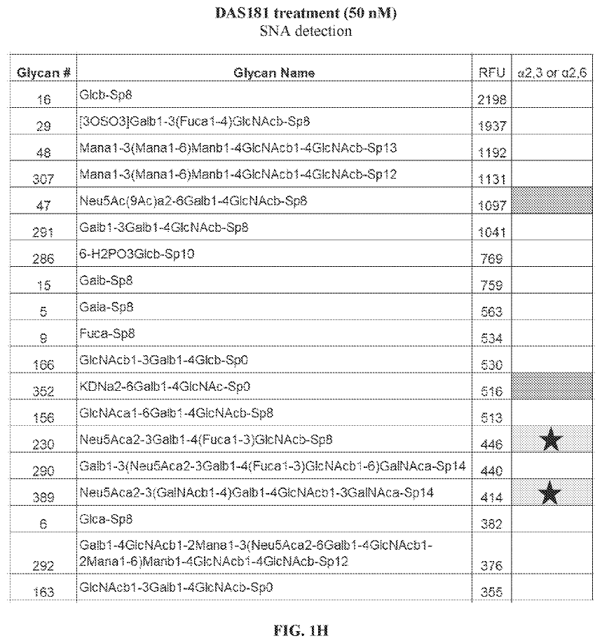

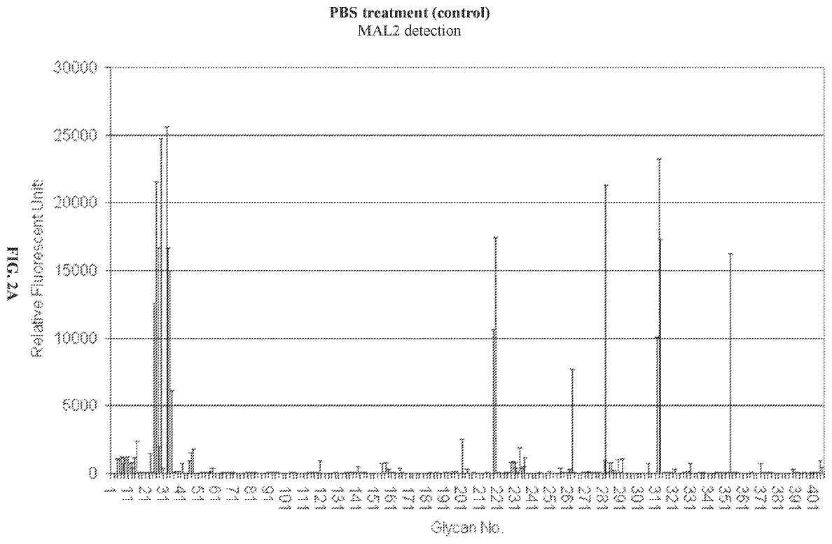

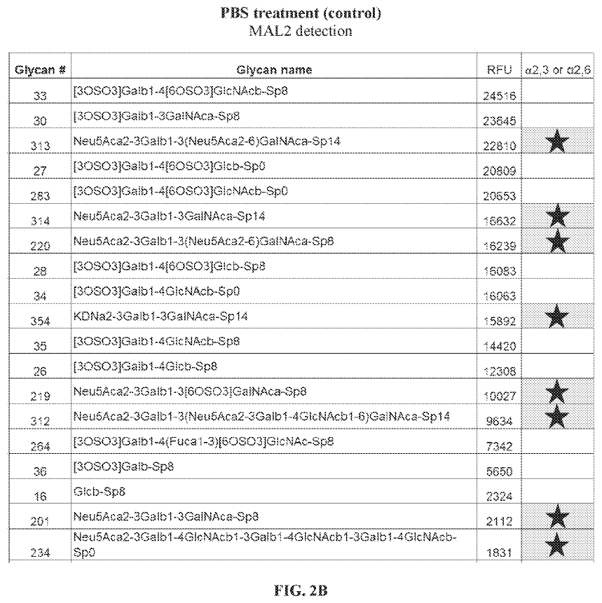

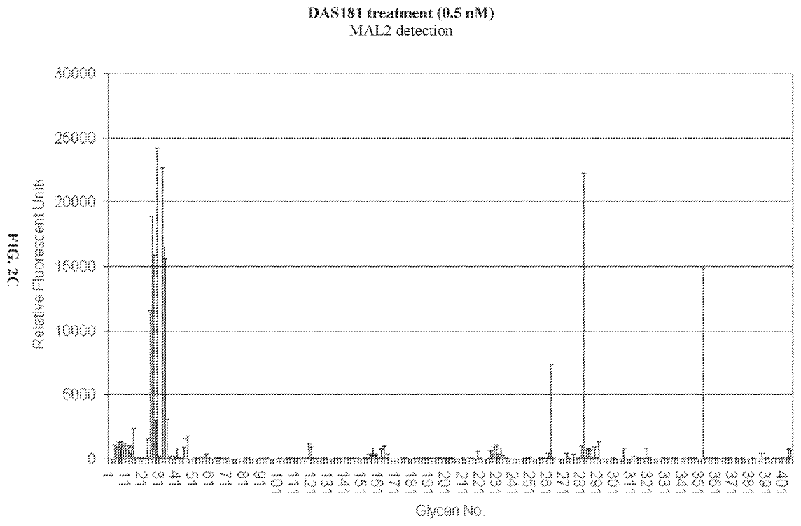

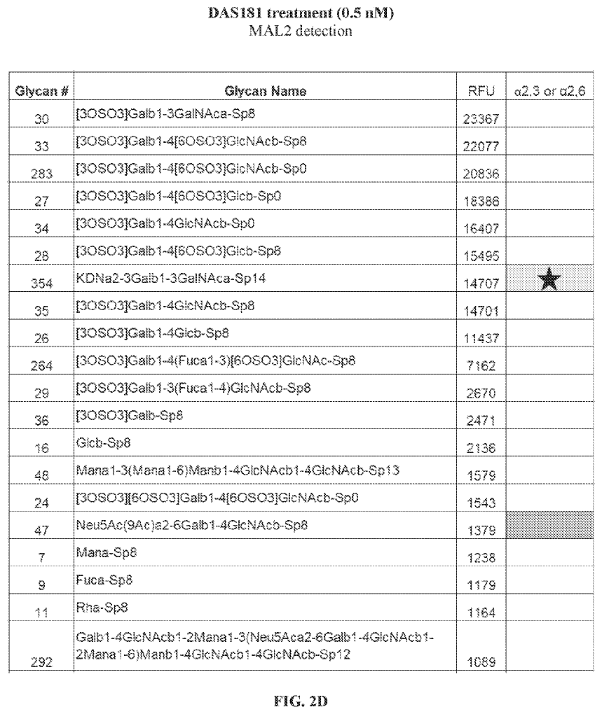

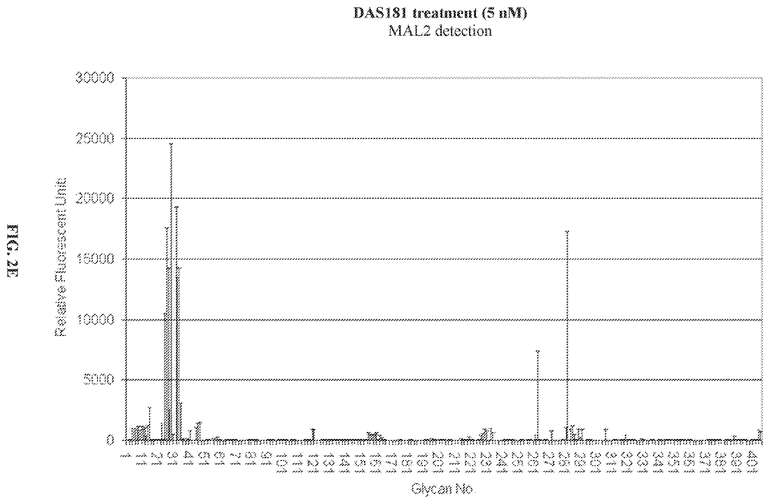

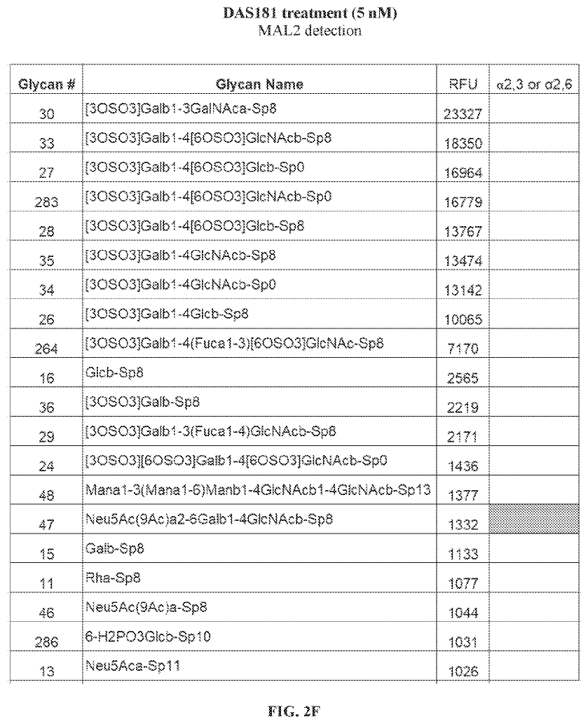

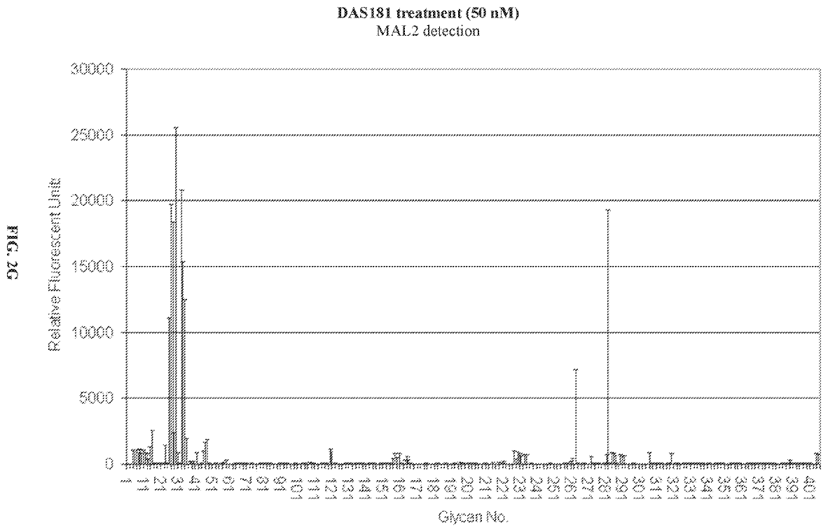

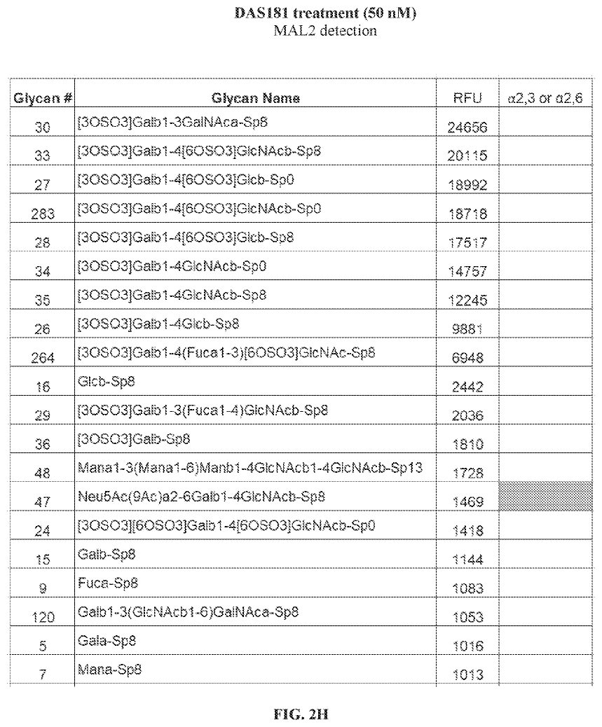

FIGS. 1A-1H and FIGS. 2A-2H provide results demonstrating removal of sialic acid as a result of DAS181 treatment. FIGS. 1A-1H show SNA-detected glycans remaining after DAS181 exposure compared to control (PBS). A synthetic substrate (CFG glycan microarray v3.2) was exposed to 0, 0.5, 5, or 50 nM DAS181 (top to bottom panels) and then remaining glycans were detected with SNA lectin. Information for the top 20 glycans detected by SNA in each graph are listed on the right; glycan number, shorthand glycan name/structure, and relative fluorescence units (RFU) are shown. Glycans with an α2,3-linked sialic acid terminus are shaded in gray and indicated with a star (right), and glycans with an α2,6-linked sialic acid terminus are shaded in gray. FIGS. 2A-2H show MAL2-detected glycans remaining after DAS181 exposure compared to control (PBS). CFG glycan microarray v3.2 was exposed to 0, 0.5, 5, or 50 nM DAS181 (top to bottom panels) and then remaining glycans were detected with MAL2 lectin. Information for the top 20 glycans detected by MAL2 in each graph are listed on the right; glycan number, shorthand glycan name/structure, and relative fluorescence units (RFU) are shown. Glycans with an α2,3-linked sialic acid terminus are shaded in gray and indicated with a star (right), glycans with an α2,6-linked sialic acid terminus are shaded in gray.

The specific activity of DAS181 against a synthetic substrate is more than 100 times higher than the activity of the human neuraminidase Neu2. This difference in specific activity is surprising because DAS181 is an engineered fusion protein yet retains high specific activity. Moreover, DAS181 efficiently cleaves sialylated glycans regardless of the structure of the more distant parts of the oligosaccharide chain (e.g. α2,3 vs. α2,6 linkage, chain length, or modification). Glycans with typical terminal sialic acid structures such as Neu5Ac (N-acetylneuraminic acid) are readily cleaved by DAS181 with near complete removal at low DAS181 concentrations (e.g., 0.5 nM). Also, glycans with KDN terminal sialic acid structure (2-keto-3-deoxynononic acid) are still cleaved by DAS181, but require higher concentrations to achieve complete removal. Residues with internal sulfate and fucosyl groups are efficiently cleaved. This surprisingly broad substrate specificity means that DAS181 can remove a variety of sialic acid types from cells; and desialylate cell surfaces of Neu5Ac and KDN terminal sialic acid structures, and from sialic acids no matter the underlying sugar structure. This broad specificity means that DAS181 has the ability to remove sialic acid residues from the surface of cancer cells much more efficiently than other sialidases. This is a discovery that was not expected, because ability to cleave sialic acids from underlying sugar structures cannot be predicted and there is no basis to believe that all Neu5Ac and KDN terminal sialic acid structures would be cleaved by one sialidase as shown in FIGS. 1A-1H and FIGS. 2A-2H.

### Example 2: Sialic Acid Removal Activity of Secreted or Transmembrane DAS181 Expressed in Cells

This example provides results demonstrating the unexpectedly high potency and broad activity of a sialidase derived from an Actinomyces viscosus sialidase (DAS181) and expressed in immune cells, wherein the sialidase is a secreted sialidase comprising an anchoring domain (a secreted, membrane-associated sialidase) or a membrane-bound sialidase comprising a transmembrane domain instead of an anchoring domain. Moreover, this example provides unexpected results demonstrating the higher potency of the Actinomyces viscosus derived sialidase DAS181 in comparison to a secreted or membrane-bound form of another sialidase, Neu2.

To evaluate the sialic acid removal activity of DAS181 expressed in cells, DNA sequences for secreted sialidase DAS181 and corresponding transmembrane sialidase catalytic domain were gene synthesized and subcloned into pcDNA3.4 and pDisplay expression vectors, respectively. In comparison, DNA sequences for DAS185 (a variant of DAS181 lacking sialidase activity due to Y348 mutation), and human Neuraminidase 2 (Neu2) were synthesized and constructed for secreted sialidase comprising an anchoring domain and transmembrane sialidase expression in the same vectors. The same anchoring domain used with the sialidase catalytic domain of Actinomyces viscosus in the design of DAS181 was combined with the sialidase sequences of DAS185 and Neu2 to generate the various secreted sialidases comprising an anchoring domain. These sialidase expression constructs were transfected into A549-red cells (A549 lung tumor cells genetically labeled with red fluorescent protein). Four days post transfection, cells were fixed and stained with fluorescently labeled Maackia Amurensisi Lectin II (MAL II), Sambucus Nigra Lectin (SNA), and Peanut Agglutinin (PNA). As discussed previously, there are two sialic acids that is most often attached to the penultimate sugar by an α-2,3 linkage or an α-2,6 linkage, which can be detected by MAL II and SNA, respectively. In addition, surface galactose exposed after sialic acid removal can be detected by PNA.

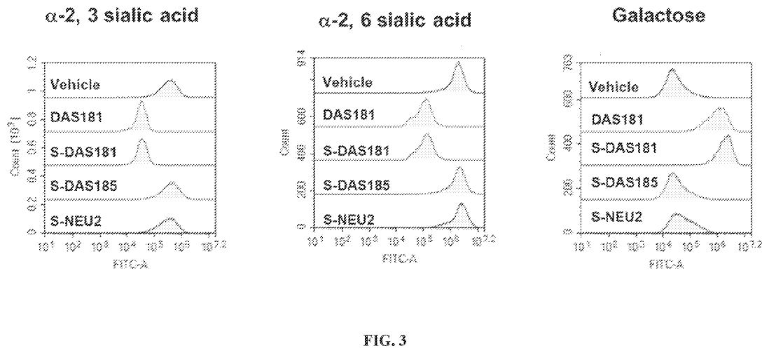

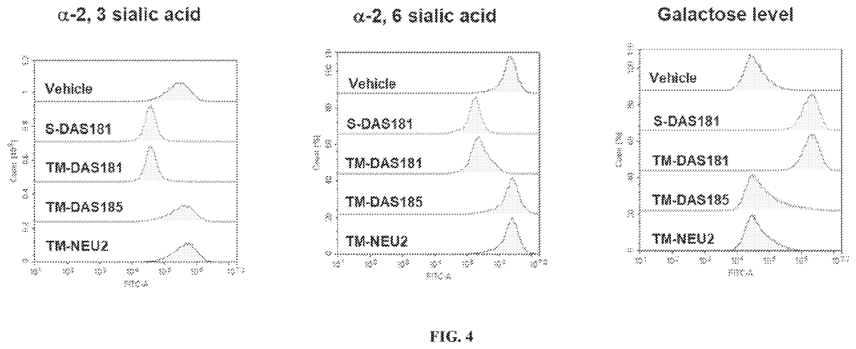

As shown in FIG. 3, transfection with secreted DAS181 construct or treatment with recombinant DAS181 resulted in substantial removal of both α-2, 3 and α-2, 6 sialic acids on cell surface while cell transfected with secreted DAS185 and Neu2 constructs still display significant levels of sialic acid staining, similar to the level observed in vehicle control treated cells. Consistent with the above results, galactose signal significantly increased after transfection with the secreted DAS181 construct and recombinant DAS181 treatment, whereas cells transfected with secreted DAS185 or Neu2 constructs showed no increase in galactose staining compared with vehicle control treated cells. FIG. 4 shows the results with transmembrane sialidase constructs, where secreted DAS181 construct was included as a positive control. Similar to what was observed with the secreted sialidase constructs, only transfection with transmembrane DAS181 construct led to significant removal of α-2, 3 and α-2, 6 sialic acids and consequent galactose exposure, whereas transfection with transmembrane DAS185 or human Neu2 constructs had little effect.

These results indicate that DAS181 either as secreted or transmembrane sialidase has substantial desialylation activity on tumor cells when expressed in cells. It is surprising that these DAS181 expression constructs when transfected in cells showed similarly potent activity to the DAS181 recombinant protein, whereas human sialidase Neu2 constructed in the same formats did not show detectable desialylation activity when transfected into cells. Therefore CAR-T cells constructed with secreted DAS181 or transmembrane DAS181 expression would be expected to have substantially greater anti-tumor activity than CAR-T cells constructed with other sialidases such as human Neu2.

### Example 3: Construction of CD19-CAR and Sialidase Expression Lentiviral Vectors

This example describes the construction of exemplary lentiviral vector constructs for expression of sialidase and/or a chimeric immune receptor (e.g., a CAR) in mammalian (e.g., human) immune cells.

To introduce transgenes into primary T and NK cells, lentiviral constructs were engineered to express a CAR (chimeric antigen receptor) recognizing CD19 (CD19-CAR), secreted sialidase comprising an anchoring domain (SP-sial) or transmembrane sialidase (TM-Sial). The CD19-CAR is designed as third generation of CAR, including CD19 scFv is from clone FMC63 (Nicholson I C, et al. Mol Immunol. 1997), CH2-CH3 spacer, CD28-TM, 41BB and CD3z. The designs of these lentiviral vectors were depicted in FIG. 5A. FIG. 5B shows the map of the pCDF1-MCS2-EF1α-copGFP Cloning and Expression Lentivector (SBI, CA) used to construct the lentivirus. Expression of CAR and Sialidase is under the transcriptional control of CMV. The expression lentiviral constructs were generated by standard DNA recombination techniques.

### Example 4: Characterization of CAR-NK Transgene Expression and Tumor Cell Killing Activity

This example provides results demonstrating enhanced tumor killing activity of a CAR-NK cell composition comprising engineered immune cells expressing a sialidase.

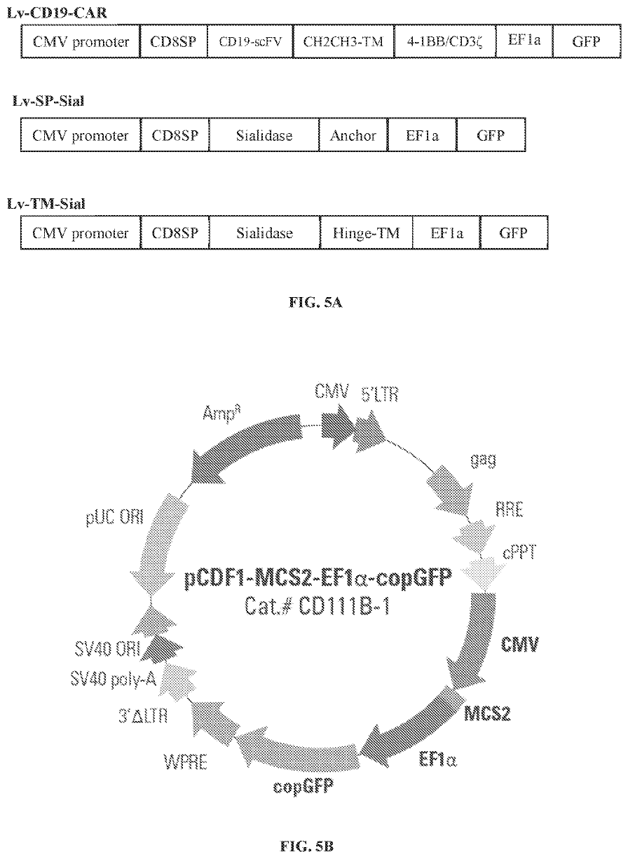

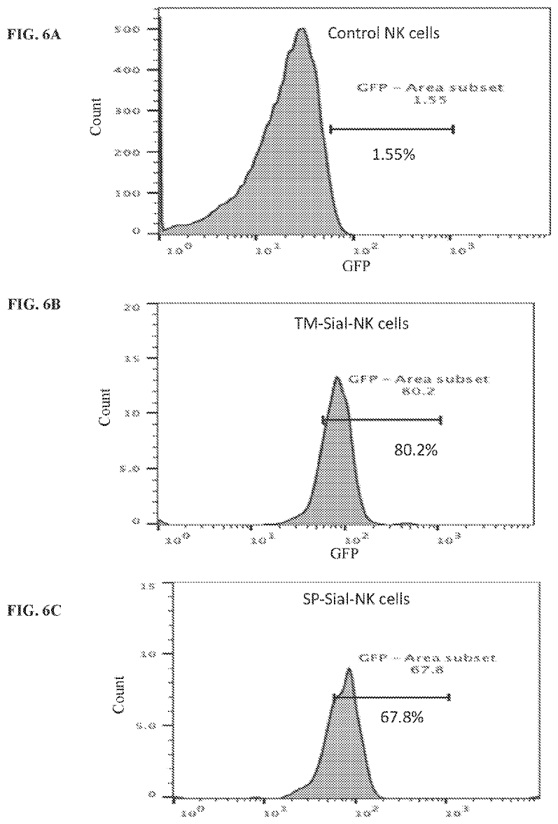

The expression of SP-sialidase (secreted sialidase comprising an anchoring domain) and TM-sialidase (sialidase comprising a transmembrane domain instead of an anchoring domain) was assessed after transduction of the sialidase lentiviral vectors (Lv-TM-Sial or Lv-SP-Sial) in human primary NK cells. Human NK cells were cultured in RPMI with 10% FBS, 1% penicillin/streptomycin/amphotericin B and 1% Glutamax in the presence of 5 mM of Rosuvastatin. Interluekin-2 (IL-2) at 200 U/ml was added to the culture medium. NK cells were transduced with lentivirus at an MOI (multiplicity of infection) of 15 and then cultured for 3 days. GFP expression by transduced NK cells were measured by flow cytometry. As shown in FIGS. 6A-6C, NK cells showed robust sialidase expression with Lv-TM-Sial or Lv-SP-Sial virus transduction efficacy at 80.2% or 67.8% respectively.

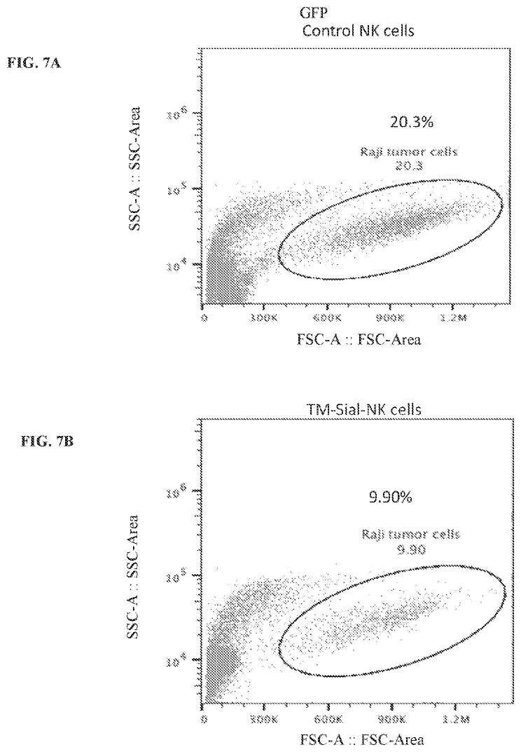

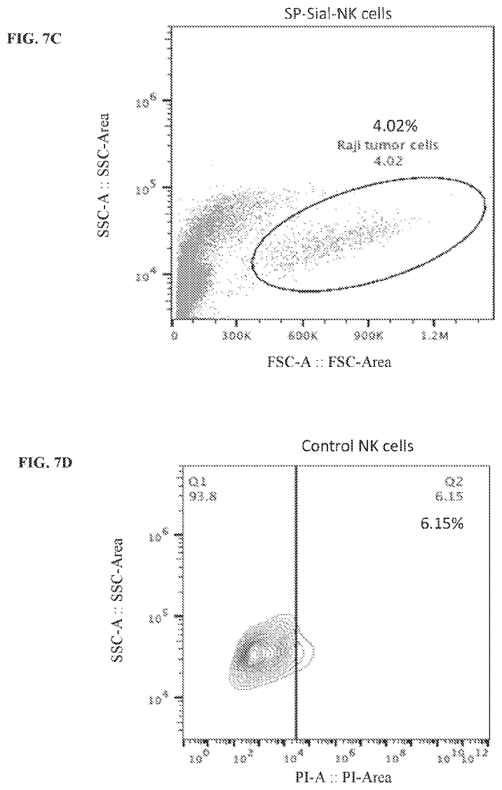

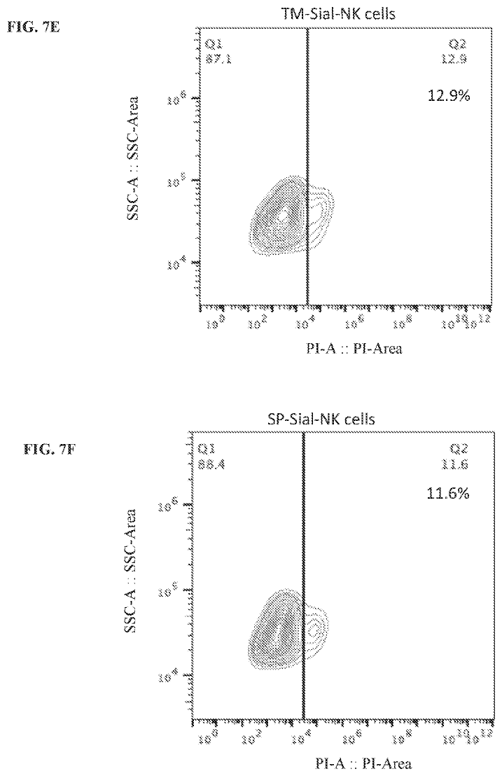

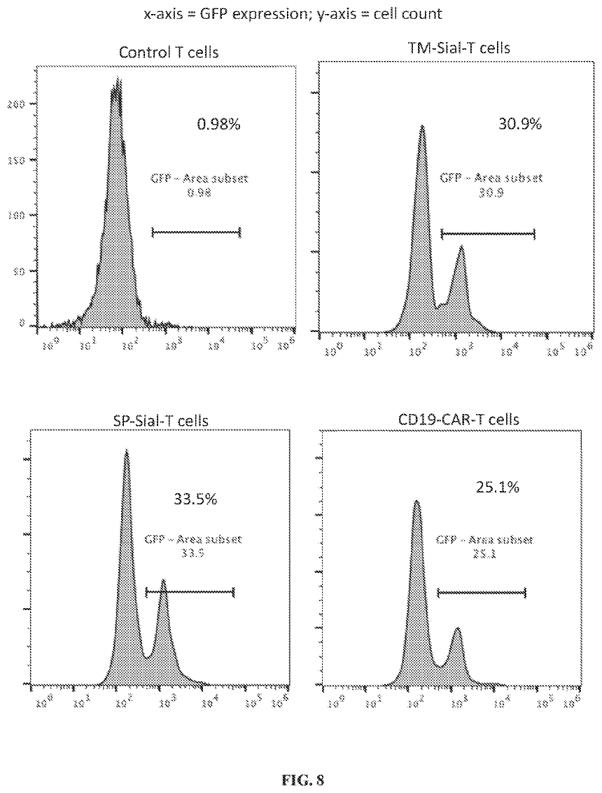

Next, the effects of Lv-Sial-NK on NK mediated tumor killing were investigated. Isolated human NK cells were transduced with lentivirus at an MOI of 15 and cultured for 3 days. CD19-CAR-NK cells were mixed with control NK cells, TM-Sial-NK cells, or SP-Sial-NK cells at 1:1 for a total of 2.5×10e4 cell per well and then cocultured with CD19+ Raji tumor cells at 1×10e4 per well in triplicates. Twenty-four hours later, the cells were collected. Pooled samples were subjected to flow analysis of live Raji tumor cells. As shown in FIG. 7A, percentage of live Raji tumor cells were reduced more significantly by TM-Sial-NK or SP-Sial-NK than by control NK cells. Moreover, Raji tumor cells were also subjected to analysis of Propidium Iodide (PI) staining as a readout for apoptosis. FIG. 7B illustrates the increased PI staining in Raji cells co-cultured with TM-Sial-NK or SP-Sial-NK, indicating significant enhancement of Raji cell apoptosis by sialidase expressing in NK cells. Both assay results support the notion that NK cells expressing Sialidase, either as secreted or transmembrane-bound form, significantly enhanced CAR-NK mediated Raji tumor cell killing.

### Example 5: Characterization of CAR-T Transgene Expression and Tumor Killing Activity

This example provides results demonstrating enhanced tumor killing activity of a CAR-T cell composition comprising engineered immune cells expressing a sialidase.

Lentiviral expression of CD19-CAR, SP-Sial, and TM-Sial in human primary T cells were also evaluated. CD3 antibody activated human T cells were cultured in RPMI with 10% FBS. IL-2 was added at 200 U/ml to the culture medium. Activated human T cells were transduced with lentivirus at an MOI of 5 and cultured for 3 days. GFP expression by lentivirus transduced human T cells were measured by flow cytometry. The results show that Lv-TM-Sial, Lv-SP-Sial virus, or Lv-CD19-CAR virus transduction efficacy were 30.9%, 33.5% or 25.1% respectively (FIG. 8).

Tumor killing activity by Sial-T cells was examined using two different readouts. Human T cells were activated with CD3 antibody and cultured in RPMI with 10% FBS, 1% penicillin/streptomycin and 1% Glutamax. IL-2 was added at 200 U/ml to the culture medium.

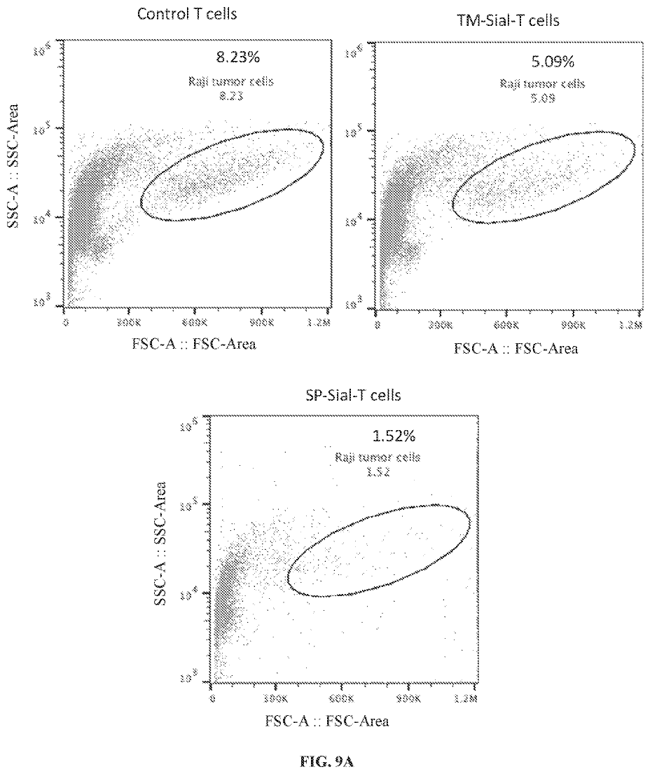

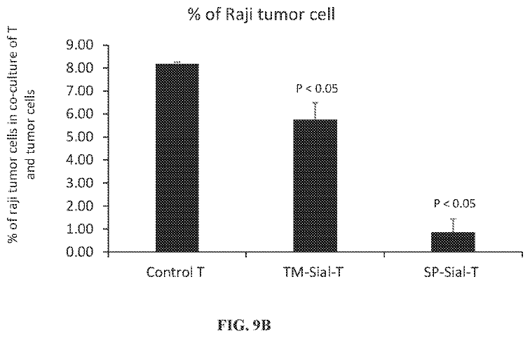

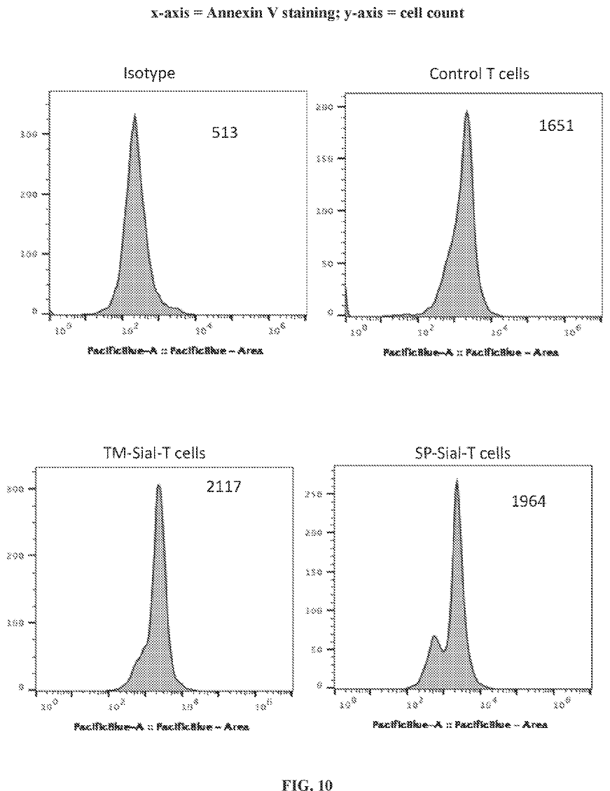

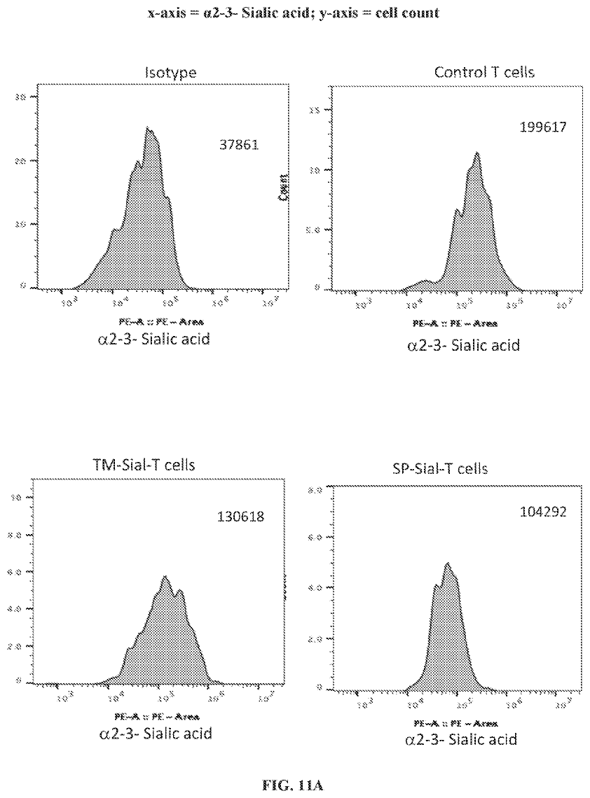

Activated human T cells were transduced with lentivirus at an MOI of 5, and then cultured for 3 days. CD19+ Raji tumor cells at 1×10e4 cells per well were co-cultured with 5×10e4 per well of CD19-CAR-T cells mixed with control T cells, TM-Sial-T cells, or SP-Sial-T cells at 1:1 ratio in triplicates. NK cells were added at 1×10e4 per well to all the wells. Twenty-four hours later, the cells were collected and subjected to flow analysis. FIG. 9A and FIG. 9B display the percentages of live Raji tumor cells cocultured with CD19-CAR-T cells, mixed with control T cells, SP-Sial-T cells or TM-Sial-T cells. The results indicate all T cells significantly induced Raji tumor cell killing in co-culture, while TM-Sial-T or SP-Sial-T further promoted reduction of live Raji cells by CD19-CAR-T cell compared to control T cells. In addition, the cells were stained with Annexin V for phosphatidylserine (PS), an early apoptosis marker and subjected to flow analysis. Raji tumor cells were gated for Annexin V analysis. FIG. 10 illustrates the MFI values of Annexin V staining of Raji cells treated with control T cells or Sial-T cells mixed with CD19-CAR-T cells.

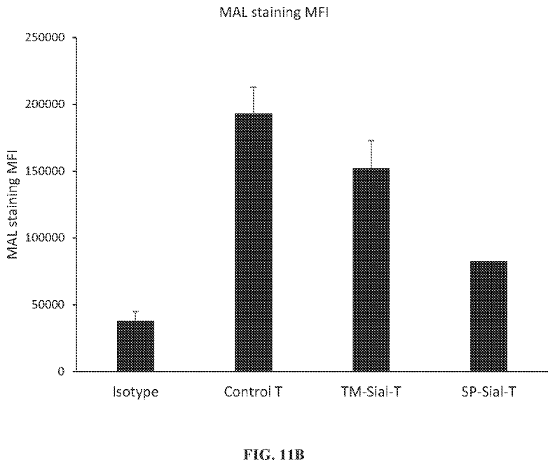

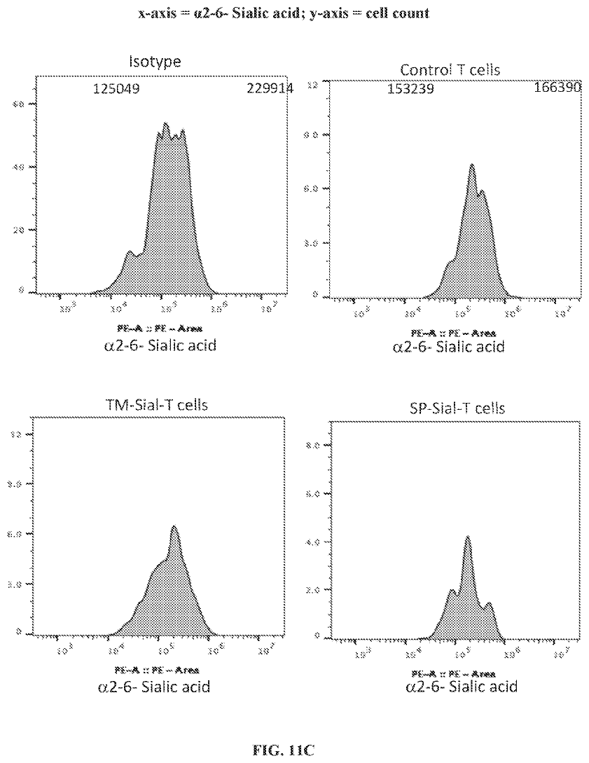

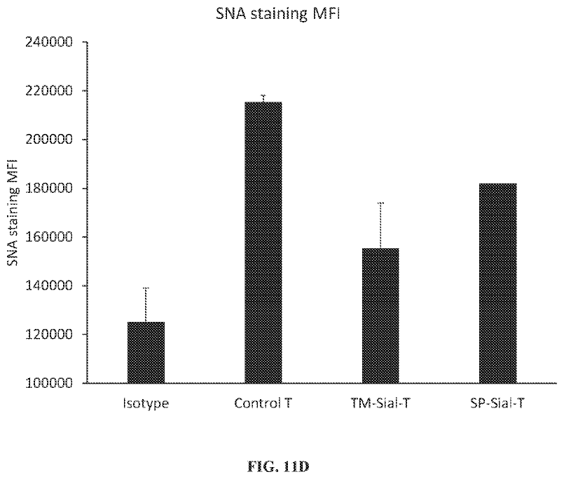

There was a slight increase in Annexin V staining intensity in cells co-cultured with Sial-T cells compared to control T cells, supporting that sialidase expression in T cells enhanced tumor cells apoptosis. Both experimental results demonstrate that sialidase expression in T cells can promote CAR-T-mediated tumor cell killing. The effects of sial-T cells on sialic acids levels on tumor cells were evaluated by staining α-2,3-linked sialic acids with MAL II or α-2.6-linked sialic acids with SNA on Raji tumors in T cell co-culture. As shown in FIGS. 11A-11D, there was significant decrease in both α-2,3- and α-2,6-linked sialic acids on Raji cells cocultured with either TM-Sial-T or SP-Sial-T compared with control-T cells, implying that sialidase may interfere with sialic acids-mediated suppression on immune cell activity.

In summary, the above study results support use of sialidase expression (e.g., an Actinomyces viscosus derived sialidase such as DAS181) to improve CAR-NK or CAR-T anti-tumor therapy.

SEQUENCE LISTING
AvCD sialidase
SEQ ID NO: 1
MGDHPQATPAPAPDASTELPASMSQAQHLAANTATDNYRI
PAITTAPNGDLLISYDERPKDNGNGGSDAPNPNHIVQRRS
TDGGKTWSAPTYIHQGTETGKKVGYSDPSYVVDHQTGTIF
NFHVKSYDQGWGGSRGGTDPENRGIIQAEVSTSTDNGWTW
THRTITADITKDKPWTARFAASGQGIQIQHGPHAGRLVQQ
YTIRTAGGAVQAVSVYSDDHGKTWQAGTPIGTGMDENKVV
ELSDGSLMLNSRASDGSGFRKVAHSTDGGQTWSEPVSDKN
LPDSVDNAQIIRAFPNAAPDDPRAKVLLLSHSPNPRPWSR
DRGTISMSCDDGASWTTSKVFHEPFVGYTTIAVQSDGSIG
LLSEDAHNGADYGGIWYRNFTMNWLGEQCGQKPAE
DAS181
SEQ ID NO: 2
MGDHPQATPAPAPDASTELPASMSQAQHLAANTATDNYRI
PAITTAPNGDLLISYDERPKDNGNGGSDAPNPNHIVQRRS
TDGGKTWSAPTYIHQGTETGKKVGYSDPSYVVDHQTGTIF
NFHVKSYDQGWGGSRGGTDPENRGIIQAEVSTSTDNGWTW
THRTITADITKDKPWTARFAASGQGIQIQHGPHAGRLVQQ
YTIRTAGGAVQAVSVYSDDHGKTWQAGTPIGTGMDENKVV
ELSDGSLMLNSRASDGSGFRKVAHSTDGGQTWSEPVSDKN
LPDSVDNAQIIRAFPNAAPDDPRAKVLLLSHSPNPRPWSR
DRGTISMSCDDGASWTTSKVFHEPFVGYTTIAVQSDGSIG
LLSEDAHNGADYGGIWYRNFTMNWLGEQCGQKPAKRKKKG
GKNGKNRRNRKKKNP
Human Neu1 sialidase
SEQ ID NO: 3
MTGERPSTALPDRRWGPRILGFWGGCRVWVFAAIFLLLSL
AASWSKAENDFGLVQPLVTMEQLLWVSGRQIGSVDTFRIP
LITATPRGTLLAFAEARKMSSSDEGAKFIALRRSMDQGST
WSPTAFIVNDGDVPDGLNLGAVVSDVETGVVFLFYSLCAH
KAGCQVASTMLVWSKDDGVSWSTPRNLSLDIGTEVFAPGP
GSGIQKQREPRKGRLIVCGHGTLERDGVFCLLSDDHGASW
RYGSGVSGIPYGQPKQENDFNPDECQPYELPDGSVVINAR
NQNNYHCHCRIVLRSYDACDTLRPRDVTFDPELVDPVVAA
GAVVTSSGIVFFSNPAHPEFRVNLTLRWSFSNGTSWRKET
VQLWPGPSGYSSLATLEGSMDGEEQAPQLYVLYEKGRNHY
TESISVAKISVYGTL
Human Neu2 sialidase
SEQ ID NO: 4
MASLPVLQKESVFQSGAHAYRIPALLYLPGQQSLLAFAEQ
RASKKDEHAELIVLRRGDYDAPTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPIQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSCAYSDLQSMGTGPDGSPLFGCLYEANDYE
EIVFLMFTLKQAFPAEYLPQ
Human Neu3 sialidase
SEQ ID NO: 5
MEEVTTCSFNSPLFRQEDDRGITYRIPALLYIPPTHTFLA
FAEKRSTRRDEDALHLVLRRGLRIGQLVQWGPLKPLMEAT
LPGHRTMNPCPVWEQKSGCVFLFFICVRGHVTERQQIVSG
RNAARLCFIYSQDAGCSWSEVRDLTEEVIGSELKHWATFA
VGPGHGIQLQSGRLVIPAYTYYIPSWFFCFQLPCKTRPHS
LMIYSDDLGVTWHHGRLIRPMVTVECEVAEPLEIPHRCQD
SSSKDAPTIQQSSPGSSLRLEEEAGTPSESWLLYSHPTSR
KQRVDLGIYLNQTPLEAACWSRPWILHCGPCGYSDLAALE
EEGLFGCLFECGTKQECEQIAFRLFTHREILSHLQGDCTS
PGRNPSQFKSN
Human Neu4 sialidase
SEQ ID NO: 6
MGVPRTPSRTVLFERERTGLTYRVPSLLPVPPGPTLLAFV
EQRLSPDDSHAHRLVLRRGTLAGGSVRWGALHVLGTAALA
EHRSMNPCPVHDAGTGTVFLFFIAVLGHTPEAVQIATGRN
AARLCCVASRDAGLSWGSARDLTEEAIGGAVQDWATFAVG
PGHGVQLPSGRLLVPAYTYRVDRRECFGKICRTSPHSFAF
YSDDHGRTWRCGGLVPNLRSGECQLAAVDGGQAGSFLYCN
ARSPLGSRVQALSTDEGTSFLPAERVASLPETAWGCQGSI
VGFPAPAPNRPRDDSWSVGPGSPLQPPLLGPGVHEPPEEA
AVDPRGGQVPGGPFSRLQPRGDGPRQPGPRPGVSGDVGSW
TLALPMPFAAPPQSPTWLLYSHPVGRRARLHMGIRLSQSP
LDPRSWTEPWVIYEGPSGYSDLASIGPAPEGGLVFACLYE
SGARTSYDEISFCTFSLREVLENVPASPKPPNLGDKPRGC
CWPS
Human Neu4 isoform 2 sialidase
SEQ ID NO: 7
MMSSAAFPRWLSMGVPRTPSRTVLFERERTGLTYRVPSLL
PVPPGPTLLAFVEQRLSPDDSHAHRLVLRRGTLAGGSVRW
GALHVLGTAALAEHRSMNPCPVHDAGTGTVFLFFIAVLGH
TPEAVQIATGRNAARLCCVASRDAGLSWGSARDLTEEAIG
GAVQDWATFAVGPGHGVQLPSGRLLVPAYTYRVDRRECFG
KICRTSPHSFAFYSDDHGRTWRCGGLVPNLRSGECQLAAV
DGGQAGSFLYCNARSPLGSRVQALSTDEGTSFLPAERVAS
LPETAWGCQGSIVGFPAPAPNRPRDDSWSVGPGSPLQPPL
LGPGVHEPPEEAAVDPRGGQVPGGPFSRLQPRGDGPRQPG
PRPGVSGDVGSWTLALPMPFAAPPQSPTWLLYSHPVGRRA
RLHMGIRLSQSPLDPRSWTEPWVIYEGPSGYSDLASIGPA
PEGGLVFACLYESGARTSYDEISFCTFSLREVLENVPASP
KPPNLGDKPRGCCWPS
Human Neu4 isoform 3 sialidase
SEQ ID NO: 8
MMSSAAFPRWLQSMGVPRTPSRTVLFERERTGLTYRVPSL
LPVPPGPTLLAFVEQRLSPDDSHAHRLVLRRGTLAGGSVR
WGALHVLGTAALAEHRSMNPCPVHDAGTGTVFLFFIAVLG
HTPEAVQIATGRNAARLCCVASRDAGLSWGSARDLTEEAI
GGAVQDWATFAVGPGHGVQLPSGRLLVPAYTYRVDRRECF
GKICRTSPHSFAFYSDDHGRTWRCGGLVPNLRSGECQLAA
VDGGQAGSFLYCNARSPLGSRVQALSTDEGTSFLPAERVA
SLPETAWGCQGSIVGFPAPAPNRPRDDSWSVGPGSPLQPP
LLGPGVHEPPEEAAVDPRGGQVPGGPFSRLQPRGDGPRQP
GPRPGVSGDVGSWTLALPMPFAAPPQSPTWLLYSHPVGRR
ARLHMGIRLSQSPLDPRSWTEPWVIYEGPSGYSDLASIGP
APEGGLVFACLYESGARTSYDEISFCTFSLREVLENVPAS
PKPPNLGDKPRGCCWPS
A. viscosus nanH sialidase
SEQ ID NO: 9
MTSHSPFSRRRLPALLGSLPLAATGLIAAAPPAHAVPTSD
GLADVTITQVNAPADGLYSVGDVMTFNITLTNTSGEAHSY
APASTNLSGNVSKCRWRNVPAGTTKTDCTGLATHTVTAED
LKAGGFTPQIAYEVKAVEYAGKALSTPETIKGATSPVKAN
SLRVESITPSSSQENYKLGDTVSYTVRVRSVSDKTINVAA
TESSFDDLGRQCHWGGLKPGKGAVYNCKPLTHTITQADVD
AGRWTPSITLTATGTDGATLQTLTATGNPINVVGDHPQAT
PAPAPDASTELPASMSQAQHLAANTATDNYRIPAIPPPPM
GTCSSPTTSARRTTATAAATTPNPNHIVQRRSTDGGKTWS
APTYIHQGTETGKKVGYSDPSYVVDHQTGTIFNFHVKSYD
QGWGGSRGGTDPENRGIIQAEVSTSTDNGWTWTHRTITAD
ITKDKPWTARFAASGQGIQIQHGPHAGRLVQQYTIRTAGG
PVQAVSVYSDDHGKTWQAGTPIGTGMDENKVVELSDGSLM
LNSRASDGSGFRKVAHSTDGGQTWSEPVSDKNLPDSVDNA
QIIRAFPNAAPDDPRAKVLLLSHSPNPRPWCRDRGTISMS
CDDGASWTTSKVFHEPFVGYTTIAVQSDGSIGLLSEDAHN
GADYGGIWYRNFTMNWLGEQCGQKPAEPSPGRRRRRHPQR
HRRRSRPRRPRRALSPRRHRHHPPRPSRALRPSRAGPGAG
AHDRSEHGAHTGSCAQSAPEQTDGPTAAPAPETSSAPAAE
PTQAPTVAPSVEPTQAPGAQPSSAPKPGATGRAPSVVNPK
ATGAATEPGTPSSSASPAPSRNAAPTPKPGMEPDEIDRPS
DGTMAQPTGAPARRVPRRRRRRRPAAGCLARDQRAADPGP
CGCRGCRRVPAAAGSPFEELNTRRAGHPALSTD
A. viscosus nanA sialidase
SEQ ID NO: 10
MTTTKSSALRRLSALAGSLALAVTGIIAAAPPAHATPTSD
GLADVTITQTHAPADGIYAVGDVMTFDITLTNTSGQARSF
APASTNLSGNVLKCRWSNVAAGATKTDCTGLATHTVTAED
LKAGGFTPQIAYEVKAVGYKGEALNKPEPVTGPTSQIKPA
SLKVESFTLASPKETYTVGDVVSYTVRIRSLSDQTINVAA
TDSSFDDLARQCHWGNLKPGQGAVYNCKPLTHTITQADAD
HGTWTPSITLAATGTDGAALQTLAATGEPLSVVVERPKAD
PAPAPDASTELPASMSDAQHLAENTATDNYRIPAITTAPN
GDLLVSYDERPRDNGNNGGDSPNPNHIVQRRSTDGGKTWS
APSYIHQGVETGRKVGYSDPSYVVDNQTGTIFNFHVKSFD
QGWGHSQAGTDPEDRSVIQAEVSTSTDNGWSWTHRTITAD
ITRDNPWTARFAASGQGIQIHQGPHAGRLVQQYTIRTADG
VVQAVSVYSDDHGQTWQAGTPTGTGMDENKVVELSDGSLM
LNSRASDGTGFRKVATSTDGGQTWSEPVPDKNLPDSVDNA
QIIRPFPNAAPSDPRAKVLLLSHSPNPRPWSRDRGTISMS
CDNGASWVTGRVFNEKFVGYTTIAVQSDGSIGLLSEDGNY
GGIWYRNFTMGWVGDQCSQPRPEPSPSPTPSAAPSAEPTS
EPTTAPAPEPTTAPSSEPSVSPEPSSSAIPAPSQSSSATS
GPSTEPDEIDRPSDGAMAQPTGGAGRPSTSVTGATSRNGL
SRTGTNALLVLGVAAAAAAGGYLVLRIRRARTE
S. oralis nanA sialidase
SEQ ID NO: 11
MNYKSLDRKQRYGIRKFAVGAASVVIGTVVFGANPVLAQE
QANAAGANTETVEPGQGLSELPKEASSGDLAHLDKDLAGK
LAAAQDNGVEVDQDHLKKNESAESETPSSTETPAEEANKE
EESEDQGAIPRDYYSRDLKNANPVLEKEDVETNAANGQRV
DLSNELDKLKQLKNATVHMEFKPDASAPRFYNLFSVSSDT
KENEYFTMSVLDNTALIEGRGANGEQFYDKYTDAPLKVRP
GQWNSVTFTVEQPTTELPHGRVRLYVNGVLSRTSLKSGNF
IKDMPDVNQAQLGATKRGNKTVWASNLQVRNLTVYDRALS
PDEVQTRSQLFERGELEQKLPEGAKVTEKEDVFEGGRNNQ
PNKDGIKSYRIPALLKTDKGTLIAGTDERRLHHSDWGDIG
MVVRRSSDNGKTWGDRIVISNPRDNEHAKHADWPSPVNID
MALVQDPETKRIFAIYDMFLESKAVFSLPGQAPKAYEQVG
DKVYQVLYKQGESGRYTIRENGEVFDPQNRKTDYRVVVDP
KKPAYSDKGDLYKGNELIGNIYFEYSEKNIFRVSNTNYLW
MSYSDDDGKTWSAPKDITHGIRKDWMHFLGTGPGTGIALR
TGPHKGRLVIPVYTTNNVSYLSGSQSSRVIYSDDHGETWQ
AGEAVNDNRPVGNQTIHSSTMNNPGAQNTESTVVQLNNGD
LKLFMRGLTGDLQVATSHDGGATWDKEIKRYPQVKDVYVQ
MSAIHTMHEGKEYILLSNAGGPGRNNGLVHLARVEENGEL
TWLKHNPIQSGKFAYNSLQELGNGEYGLLYEHADGNQNDY
TLSYKKFNWDFLSRDRISPKEAKVKYAIQKWPGIIAMEFD
SEVLVNKAPTLQLANGKTATFMTQYDTKTLLFTIDPEDMG
QRITGLAEGAIESMHNLPVSLAGSKLSDGINGSEAAIHEV
PEFTGGVNAEEAAVAEIPEYTGPLATVGEEVAPTVEKPEF
TGGVNAEEAPVAEMPEYTGPLSTVGEEVAPTVEKPEFTGG
VNAVEAAVHELPEFKGGVNAVLAASNELPEYRGGANFVLA
ASNDLPEYIGGVNGAEAAVHELPEYKGDTNLVLAAADNKL
SLGQDVTYQAPAAKQAGLPNTGSKETHSLISLGLAGVLLS
LFAFGKKRKE
S. oralis nanH sialidase
SEQ ID NO: 12
MSDLKKYEGVIPAFYACYDDQGEVSPERTRALVQYFIDKG
VQGLYVNGSSGECIYQSVEDRKLILEEVMAVAKGKLTIIA
HVACNNTKDSMELARHAESLGVDAIATIPPIYFRLPEYSV
AKYWNDISAAAPNTDYVIYNIPQLAGVALTPSLYTEMLKN
PRVIGVKNSSMPVQDIQTFVSLGGEDHIVFNGPDEQFLGG
RLMGAKAGIGGTYGAMPELFLKLNQLIAEKDLETARELQY
AINAIIGKLTSAHGNMYGVIKEVLKINEGLNIGSVRSPLT
PVTEEDRPVVEAAAQLIRETKERFL
S. mitis nanA sialidase
SEQ ID NO: 13
MNQRHFDRKQRYGIRKFTVGAASVVIGAVVFGVAPALAQE
APSTNGETAGQSLPELPKEVETGNLTNLDKELADKLSTAT
DKGTEVNREELQANPGSEKAAETEASNETPATESEDEKED
GNIPRDFYARELENVNTVVEKEDVETNPSNGQRVDMKEEL
DKLKKLQNATIHMEFKPDASAPRFYNLFSVSSDTKVNEYF
TMAILDNTAIVEGRDANGNQFYGDYKTAPLKIKPGEWNSV
TFTVERPNADQPKGQVRVYVNGVLSRTSPQSGRFIKDMPD
VNQVQIGTTKRTGKNFWGSNLKVRNLTVYDRALSPEEVKK
RSQLFERGELEKKLPEGAKVTDKLDVFQGGENRKPNKDGI
ASYRIPALLKTDKGTLIAGADERRLHHSDWGDIGMVVRRS
DDKGKTWGDRIVISNPRDNENARRAHAGSPVNIDMALVQD
PKTKRIFSIFDMFVEGEAVRDLPGKAPQAYEQIGNKVYQV
LYKKGEAGHYTIRENGEVFDPENRKTEYRVVVDPKKPAYS
DKGDLYKGEELIGNVYFDYSDKNIFRVSNTNYLWMSYSDD
DGKTWSAPKDITYGIRKDWMHFLGTGPGTGIALHSGPHKG
RLVIPAYTTNNVSYLGGSQSSRVIYSDDHGETWHAGEAVN
DNRPIGNQTIHSSTMNNPGAQNTESTVVQLNNGDLKLFMR
GLTGDLQVATSKDGGATWEKDVKRYADVKDVYVQMSAIHT
VQEGKEYIILSNAGGPGRYNGLVHVARVEANGDLTWIKHN
PIQSGKFAYNSLQDLGNGEFGLLYEHATATQNEYTLSYKK
FNWDFLSKDGVAPTKATVKNAVEMSKNVIALEFDSEVLVN
QPPVLKLANGNFATFLTQYDSKTLLFAASKEDIGQEITEI
IDGAIESMHNLPVSLEGAGVPGGKNGAKAAIHEVPEFTGA
VNGEGTVHEDPAFEGGINGEEAAVHDVPDFSGGVNGEVAA
IHEVPEFTGGINGEEAAKLELPSYEGGANAVEAAKSELPS
YEGGANAVEAAKLELPSYESGAHEVQPASSNLPTLADSVN
KAEAAVHKGKEYKANQSTAVQAMAQEHTYQAPAAQQHLLP
KTGSEDKSSLAIVGFVGMFLGLLMIGKKRE
S. mitis nanA_1 sialidase
SEQ ID NO: 14
MNQSSLNRKNRYGIRKFTIGVASVAIGSVLFGITPALAQE
TTTNIDVSKVETSLESGAPVSEPVTEVVSGDLNHLDKDLA
DKLALATNQGVDVNKHNLKEETSKPEGNSEHLPVESNTGS
EESIEHHPAKIEGADDAVVPPRDFFARELTNVKTVFERED
LATNTGNGQRVDLAEELDQLKQLQNATIHMEFKPDANAPQ
FYNLFSVSSDKKKDEYFSMSVNKGTAMVEARGADGSHFYG
SYSDAPLKIKPGQWNSVTFTVERPKADQPNGQVRLYVNGV
LSRTNTKSGRFIKDMPDVNKVQIGATRRANQTMWGSNLQI
RNLTVYNRALTIEEVKKRSHLFERNDLEKKLPEGAEVTEK
KDIFESGRNNQPNGEGINSYRIPALLKTDKGTLIAGGDER
RLHHFDYGDIGMVIRRSQDNGKTWGDKLTISNLRDNPEAT
DKTATSPLNIDMVLVQDPTTKRIFSIYDMFPEGRAVFGMP
NQPEKAYEEIGDKTYQVLYKQGETERYTLRDNGEIFNSQN
KKTEYRVVVNPTEAGFRDKGDLYKNQELIGNIYFKQSDKN
PFRVANTSYLWMSYSDDDGKTWSAPKDITPGIRQDWMKFL
GTGPGTGIVLRTGAHKGRILVPAYTTNNISHLGGSQSSRL
IYSDDHGQTWHAGESPNDNRPVGNSVIHSSNMNKSSAQNT
ESTVLQLNNGDVKLFMRGLTGDLQVATSKDGGVTWEKTIK
RYPEVKDAYVQMSAIHTMHDGKEYILLSNAAGPGRERKNG
LVHLARVEENGELTWLKHNPIQNGEFAYNSLQELGGGEYG
LLYEHRENGQNYYTLSYKKFNWDFVSKDLISPTEAKVSQA
YEMGKGVFGLEFDSEVLVNRAPILRLANGRTAVFMTQYDS
KTLLFAVDKKDIGQEITGIVDGSIESMHNLTVNLAGAGIP
GGMNAAESVEHYTEEYTGVLGTSGVEGVPTISVPEYEGGV
NSELALVSEKEDYRGGVNSASSVVTEVLEYTGPLSTVGSE
DAPTVSVLEYEGGVNIDSPEVTEAPEYKEPIGTSGYELAP
TVDKPAYTGTIEPLEKEENSGAIIEEGNVSYITENNNKPL
ENNNVTTSSIISESSKLKHTLKNATGSVQIHASEEVLKNV
KDVKIQEVKVSSLSSLNYKAYDIQLNDASGKAVQPKGTVI
VTFAAEQSVENVYYVDSKGNLHTLEFLQKDGEVTFETNHF
SIYAMTFQLSLDNVVLDNHREDKNGEVNSASPKLLSINGH
SQSSQLENKVSNNEQSKLPNTGEDKSISTVLLGFVGVILG
AMIFYRRKDSEG
S. mitis nanA 2 sialidase
SEQ ID NO: 15
MDKKKIILTSLASVAVLGAALAASQPSLVKAEEQPTASQP
AGETGTKSEVTSPEIKQAEADAKAAEAKVTEAQAKVDTTT
PVADEAAKKLETEKKEADEADAAKTKAEEAKKTADDELAA
AKEKAAEADAKAKEEAKKEEDAKKEEADSKEALTEALKQL
PDNELLDKKAKEDLLKAVEAGDLKASDILAELADDDKKAE
ANKETEKKLRNKDQANEANVATTPAEEAKSKDQLPADIKA
GIDKAEKADAARPASEKLQDKADDLGENVDELKKEADALK
AEEDKKAETLKKQEDTLXEAKEALKSAKDNGFGEDITAPL
EKAVTAIEKERDAAQNAFDQAASDTKAVADELNKLTDEYN
KTLEEVKAAKEKEANEPAKPVEEEPAKPAEKTEAEKAAEA
KTEADAKVAELQKKADEAKTKADEATAKATKEAEDVKAAE
KAKEEADKAKTDAEAELAKAKEEAEKAKAKVEELKKEEKD
NLEALKAALDQLEKDIDADATITNKEEAKKALGKEDILAA
VEKGDLTAGDVLKELENQNATAEATKDQDPQADEIGATKQ
EGKPLSELPAADKEKLDAAYNKEASKPIVKKLQDIADDLV
EKIEKLTKVADKDKADATEKAKAVEEKNAALDKQKETLDK
AKAALETAKKNQADQAIQDGLQDAVTKLEASFASAKTAAD
EAQAKFDEVNEVVKAYKAAIDELTDDYNATLGHIENLKEV
PKGEEPKDFSGGVNDDEAPSSTPNTNEFTGGANDADAPTA
PNANEFAGGVNDEEAPTTENKPEFNGGVNDEEAPTVPNKP
EGEAPKPTGENAKDAPVVKLPEFGANNPEIKKILDEIAKV
KEQIKDGEENGSEDYYVEGLKERLADLEEAFDTLSKNLPA
VNKVPEYTGPVTPENGQTQPAVNTPGGQQGGSSQQTPAVQ
QGGSGQQAPAVQQGGSNQQVPAVQQTNTPAVAGTSQDNTY
QAPAAKEEDKKELPNTGGQESAALASVGFLGLLLGALPFV
KRKN
S. mitis nanA 3 sialidase
SEQ ID NO: 16
MKYRDFDRKRRYGIRKFAVGAASVVIGTVVFGANPVLAQE
QANAAGANTETVEPGQGLSELPKEASSGDLAHLDKDLAGK
LAAAQDNGVEVDQDHLKKNESAESETPSSTETPAEGTNKE
EESEDQGAIPRDYYSRDLKNANPVLEKEDVETNAANGQRV
DLSNELDKLKQLKNATVHMEFKPDASAPRFYNLFSVSSDT
KENEYFTISVLDNTALIEGRGANGEQFYDKYTDAPLKVRP
GQWNSVTFTVEQPTTELPHGRVRLYVNGVLSRTSLKSGNF
IKDMPDVNQAQLGATKRGNKTVWASNLQVRNLTVYDRALS
PDEVQTRSQLFERGELEQKLPEGAKVTEKEDVFEGGRNNQ
PNKDGIKSYRIPALLKTDKGTLIAGTDERRLHHSDWGDIG
MVVRRSSDNGKTWGDRIVISNPRDNEHAKHADWPSPVNID
MALVQDPETKRIFAIYDMFLESKAVFSLPGQAPKAYEQVG
DKVYQVLYKQGESGRYTIRENGEVFDPQNRKTDYRVVVDP
KKPAYSDKGDLYKGNELIGNIYFEYSEKNIFRVSNTNYLW
MSYSDDDGKTWSAPKDITHGIRKDWMHFLGTGPGTGIALR
TGPHKGRLVIPVYTTNNVSYLSGSQSSRVIYSDDHGETWQ
AGEAVNDNRPVGNQTIHSSTMNNPGAQNTESTVVQLNNGD
LKLFMRGLTGDLQVATSHDGGATWDKEIKRYPQVKDVYVQ
MSAIHTMHEGKEYILLSNAGGPGRNNGLVHLARVEENGEL
TWLKHNPIQSGKFAYNSLQDLGNGEYGLLYEHADGNQNDY
TLSYKKFNWDFLTKDWISPKEAKVKYAIEKWPGILAMEFD
SEVLVNKAPTLQLANGKTARFMTQYDTKTLLFTVDSEDMG
QKVTGLAEGAIESMHNLPVSVAGTKLSNGMNGSEAAVHEV
PEYTGPLGTAGEEPAPTVEKPEFTGGVNGEEAAVHEVPEY
TGPLGTSGEEPAPTVEKPEFTGGVNAVEAAAHEVPEYTGP
LGTSGKEPAPTVEKPEYTGGVNAVEAAVHEVPEYTGPLAT
VGEEAAPKVDKPEFTGGVNAVEAAVHELPEYTGGVNAADA
AVHEIAEYKGADSLVTLAAEDYTYKAPLAQQTLPDTGNKE
SSLLASLGLTAFFLGLFAMGKKREK
S. mitis nanA 4 sialidase
SEQ ID NO: 17
MEKIWREKSCRYSIRKLTVGTASVLLGAVFLASHTVSADT
IKVKQNESTLEKTTAKTDTVTKTTESTEHTQPSEAIDHSK
QVLANNSSSESKPTEAKVASATTNQASTEAIVKPNENKET
EKQELPVTEQSNYQLNYDRPTAPSYDGWEKQALPVGNGEM
GAKVFGLIGEERIQYNEKTLWSGGPRPDSTDYNGGNYRER
YKILAEIRKALEDGDRQKAKRLAEQNLVGPNNAQYGRYLA
FGDIFMVFNNQKKGLDTVTDYHRGLDITEATTTTSYTQDG
TTFKRETFSSYPDDVTVTHLTQKGDKKLDFTVWNSLTEDL
LANGDYSAEYSNYKSGHVTTDPNGILLKGTVKDNGLQFAS
YLGIKTDGKVTVHEDSLTITGASYATLLLSAKTNFAQNPK
TNYRKDIDLEKTVKGIVEAAQGKYYETLKRNHIKDYQSLF
NRVKLNLGGSNIAQTTKEALQTYNPTKGQKLEELFFQYGR
YLLISSSRDRTDALPANLQGVWNAVDNPPWNADYHLNVNL
QMNYWPAYMSNLAETAKPMINYIDDMRYYGRIAAKEYAGI
ESKDGQENGWLVHTQATPFGWTTPGWNYYWGWSPAANAWM
MQNVYDYYKFTKDETYLKEKIYPMLKETAKFWNSFLHYDQ
ASDRWVSSPSYSPEHGTITIGNTFDQSLVWQLFHDYMEVA
NHLNVDKDLVTEVKAKFDKLKPLHINKEGRIKEWYEEDSP
QFTNEGIENNHRHVSHLVGLFPGTLFSKDQAEYLEAARAT
LNHRGDGGTGWSKANKINLWARLLDGNRAHRLLAEQLKYS
TLENLWDTHAPFQIDGNFGATSGIAEMLLQSHTGYIAPLP
ALPDAWKDGQVSGLVARGNFEVSMQWKDKNLQSLSFLSNV
GGDLVVDYPNIEASQVKVNGKPVKATVLKDGRIQLATQKG
DVITFEHFSGRVTSLTAVRQNGVTAELTFNQVEGATHYVI
QRQVKDESGQTSATREFVTNQTHFIDRSLDPQLAYTYTVK
AMLGNVSTQVSEKANVETYNQLMDDRDSRIQYGSAFGNWA
DSELFGGTEKFADLSLGNYTDKDATATIPFNGVGIEIYGL
KSSQLGIAEVKIDGKSVGELDFYTAGATEKGSLIGRFTGL
SDGAHVMTITVKQEHKHRGSERSKISLDYFKVLPGQGTTI
EKMDDRDSRIQYGSQFKDWSDTELYKSTEKYADINNSDPS
TASEAQATIPFTGTGIRIYGLKTSALGKALVTLDGKEMPS
LDFYTAGATQKATLIGEFTNLTDGNHILTLKVDPNSPAGR
KKISLDSFDVIKSPAVSLDSPSIAPLKKGDKNISLTLPAG
DWEAIAVTFPGIKDPLVLRRIDDNHLVTTGDQTVLSIQDN
QVQIPIPDETNRKIGNAIEAYSIQGNTTSSPVVAVFTKKD
EKKVENQQPTTSKGDDPAPIVEIPEYTKPIGTAGLEQPPT
VSIPEYTQPIGTAGLEQAPTVSIPEYTKPVGTAGIEQAPT
VSIPEYTKPIGTAGLEQAPTVSIPEYTQPIGTAGLEQPPT
VSIPEYTKSIGTAGLEQPPVVNVPEYTQPIGTAGIEQPPT
VSIPEYTKPIGTAGQEQALTVSIPEYTKPIGTAGQEQAPT
VSVPEYKLRVLKDERTGVEIIGGATDLEGISHISSRRVLA
QELFGKTYDAYDLHLKNSTDQSLQPKGSVLVRLPISSAVE
NVYYLTPSKELQALDFTIREGMAEFTTSHFSTYAVVYQAN
GASTTAEQKPSETDIKPLANSSEQVSSSPDLVQSTNDSPK
EQLPATGETSNPLLFLSGLSLVLTATFLLKSKKDESN
S. mitis nanA 5 sialidase
SEQ ID NO: 18
MKQYFLEKGRIFSIRKLTVGVASVAVGLTFFASGNVAASE
LVTEPKLEVDGQSKEVADVKHEKEEAVKEEAVKEEVTEKT
ELTAEKATEEAKTAEVAGDVLPEEIPDRAYPDTPVKKVDT
AAIVSEQESPQVETKSILKPTEVAPTEGEKENRAVINGGQ
DLKRINYEGQPATSAAMVYTIFSSPLAGGGSQRYLNSGSG
IFVAPNIMLTVAHNFLVKDADTNAGSIRGGDTTKFYYNVG
SNTAKNNSLPTSGNTVLFKEKDIHFWNKEKFGEGIKNDLA
LVVAPVPLSIASPNKAATFTPLAEHREYKAGEPVSTIGYP
TDSTSPELKEPIVPGQLYKADGVVKGTEKLDDKGAVGITY
RLTSVSGLSGGGIINGDGKVIGIHQHGTVDNMNIAEKDRF
GGGLVLSPEQLAWVKEIIDKYGVKGWYQGDNGNRYYFTPE
GEMIRNKTAVIGKNKYSFDQNGIATLLEGVDYGRVVVEHL
DQKDNPVKENDTFVEKTEVGTQFDYNYKTEIEKTDFYKKN
KEKYEIVSIDGKAVNKQLKDTWGEDYSVVSKAPAGTRVIK
VVYKVNKGSFDLRYRLKGTDQELAPATVDNNDGKEYEVSF
VHRFQAKEITGYRAVNASQEATIQHKGVNQVIFEYEKIED
PKPATPATPVVDPKDEETEIGNYGPLPSKAQLDYHKEELA
AFIHYGMNTYTNSEWGNGRENPQNFNPTNLDTDQWIKTLK
DAGFKRTIMVVKHHDGFVIYPSQYTKHTVAASPWKDGKGD
LLEEISKSATKYDMNMGVYLSPWDANNPKYHVSTEKEYNE
YYLNQLKEILGNPKYGNKGKFIEVWMDGARGSGAQKVTYT
FDEWFKYIKKAEGDIAIFSAQPTSVRWIGNERGIAGDPVW
HKVKKAKITDDVKNEYLNHGDPEGDMYSVGEADVSIRSGW
FYHDNQQPKSIKDLMDIYFKSVGRGTPLLLNIPPNKEGKF
ADADVARLKEFRATLDQMYATDFAKGATVTASSTRKNHLY
QASNLTDGKDDTSWALSNDAKTGEFTVDLGQKRRFDVVEL
KEDIAKGQRISGFKVEVELNGRWVPYGEGSTVGYRRLVQG
QPVEAQKIRVTITNSQATPILTNFSVYKTPSSIEKTDGYP
LGLDYHSNTTADKANTTWYDESEGIRGTSMWTNKKDASVT
YRFNGTKAYVVSTVDPNHGEMSVYVDGQKVADVQTNNAAR
KRSQMVYETDDLAPGEHTIKLVNKTGKAIATEGIYTLNNA
GKGMFELKETTYEVQKGQPVTVTIKRVGGSKGAATVHVVT
EPGTGVHGKVYKDTTADLTFQDGETEKTLTIPTIDFTEQA
DSIFDFKVKMTSASDNALLGFASEATVRVMKADLLQKDQV
SHDDQASQLDYSPGWHHETNSAGKYQNTESWASFGRLNEE
QKKNASVTAYFYGTGLEIKGFVDPGHGIYKVTLDGKELEY
QDGQGNATDVNGKKYFSGTATTRQGDQTLVRLTGLEEGWH
AVTLQLDPKRNDTSRNIGIQVDKFITRGEDSALYTKEELV
QAMKNWKDELAKFDQTSLKNTPEARQAFKSNLDKLSEQLS
ASPANAQEILKIATALQAILDKEENYGKEDTPTSEQPEEP
NYDKAMASLSEAIQNKSKELSSDKEAKKKLVELSEQALTA
IQEAKTQDAVDKALQAALTSINQLQATPKEEVKPSQPEEP
NYDKAMASLAEAIQNKSKELGSDKESKKKLVELSEQALTA
IQEAKTQDAVDKALQAALTSINQLQATPKEEAKPSQPEEP
NYDKAMASLAEAIQNKSKELGSDKEAKKKLVELSEQALTA
IQEAKTQDAVDKALQAALTSINQLQATPKEEVKHSIVPTD
GDKELVQPQPSLEVVEKVINFKKVKQEDSSLPKGETRVTQ
VGRAGKERILTEVAPDGSRTIKLREVVEVAQDEIVLVGTK
KEESGKIASSVHEVPEFTGGVIDSEATIHNLPEFTGGVTD
SEAAIHNLPEFTGGVTDSEAAIHNLPEFTGGMTDSEAAIH
NLPEFTGGMTDSEGVAHGVSNVEEGVPSGEATSHQESGFT
SDVTDSETTMNEIVYKNDEKSYVVPPMLEDKTYQAPANRQ
EVLPKTGSEDGSAFASVGIIGMFLGMIGIVKRKKD
S. mitis nanH sialidase
SEQ ID NO: 19
MSGLKKYEGVIPAFYACYDDAGEVSPERTRALVQYFIDKG
VQGLYVNGSSGECIYQSVEDRKLILEEVMAVAKGKLTIIA
HVACNNTKDSIELARHAESLGVDAIATIPPIYFRLPEYSV
AKYWNDISAAAPNTDYVIYNIPQLAGVALTPSLYTEMLKN
PRVIGVKNSSMPVQDIQTFVSLGGDDHIVFNGPDEQFLGG
RLMGAKAGIGGTYGAMPELFLKLNQLIADKDLETARELQY
AINAIIGKLTAAHGNMYCVIKEVLKINEGLNIGSVRSPLT
PVTEEDRPVVEAAAQLIRESKERFL
P. gingivalis sialidase
SEQ ID NO: 20
MANNTLLAKTRRYVCLVVFCCLMAMMHLSGQEVTMWGDSH
GVAPNQVRRTLVKVALSESLPPGAKQIRIGFSLPKETEEK
VTALYLLVSDSLAVRDLPDYKGRVSYDSFPISKEDRTTAL
SADSVAGRCFFYLAADIGPVASFSRSDTLTARVEELAVDG
RPLPLKELSPASRRLYREYEALFVPGDGGSRNYRIPSILK
TANGTLIAMADRRKYNQTDLPEDIDIVMRRSTDGGKSWSD
PRIIVQGEGRNHGFGDVALVQTQAGKLLMIFVGGVGLWQS
TPDRPQRTYISESRDEGLTWSPPRDITHFIFGKDCADPGR
SRWLASFCASGQGLVLPSGRVMFVAAIRESGQEYVLNNYV
LYSDDEGGTWQLSDCAYHRGDEAKLSLMPDGRVLMSVRNQ
GRQESRQRFFALSSDDGLTWERAKQFEGIHDPGCNGAMLQ
VKRNGRNQMLHSLPLGPDGRRDGAVYLFDHVSGRWSAPVV
VNSGSSAYSDMTLLADGTIGYFVEEDDEISLVFIRFVLDD
LFDARQ
T. forsythia siaHI sialidase
SEQ ID NO: 21
MTKKSSISRRSFLKSTALAGAAGMVGTGGAATLLTSCGGG
ASSNENANAANKPLKEPGTYYVPELPDMAADGKELKAGII
GCGGRGSGAAMNFLAAANGVSIVALGDTFQDRVDSLAQKL
KDEKNIDIPADKRFVGLDAYKQVIDSDVDVVIVATPPNFR
PIHFQYAVEKSKHCFLEKPICVDAVGYRTIMATAKQAQAK
NLCVITGTQRHHQRSYIASYQQIMNGAIGEITGGTVYWNQ
SMLWYRERQAGWSDCEWMIRDWVNWKWLSGDHIVEQHVHN
IDVFTWFSGLKPVKAVGFGSRQRRITGDQYDNFSIDFTME
NGIHLHSMCRQIDGCANNVSEFIQGTKGSWNSTDMGIKDL
AGNVIWKYDVEAEKASFKQNDPYTLEHVNWINTIRAGKSI
DQASETAVSNMAAIMGRESAYTGEETTWEAMTAAALDYTP
ADLNLGKMDMKPFVVPVPGKPLEKK
T. forsythia nanH sialidase
SEQ ID NO: 22
MKKFFWIIGLFISMLTTRAADSVYVQNPQIPILIDRTDNV
LFRIRIPDATKGDVLNRLTIRFGNEDKLSEVKAVRLFYAG
TEAGTKGRSRFAPVTYVSSHNIRNTRSANPSYSVRQDEVT
TVANTLTLKTRQPMVKGINYFWVSVEMDRNTSLLSKLTPT
VTEAVINDKPAVIAGEQAAVRRMGIGVRHAGDDGSASFRI
PGLVTTNEGTLLGVYDVRYNNSVDLQEHIDTHGMGNARAW
TNSMPGMTPDETAQLMMVKSTDDGRTWSEPINITSQVKDP
SWCFLLQGPGRGITMRDGTLVFPIQFIDSLRVPHAGIMYS
KDRGETWHIHQPARTNTTEAQVAEVEPGVLMLNMRDNRGG
SRAVSITRDLGKSWTEHSSNRSALPESICMASLISVKAKD
NIIGKDLLFFSNPNTTEGRHHITIKASLDGGVTWLPAHQV
LLDEEDGWGYSCLSMIDRETVGIFYESSVAHMTFQAVKIK
DLIR
A. muciniphila sialidase
SEQ ID NO: 23
MTWLLCGRGKWNKVKRMMNSVFKCLMSAVCAVALPAFGQE
EKTGFPTDRAVTVFSAGEGNPYASIRIPALLSIGKGQLLA
FAEGRYKNTDQGENDIIMSVSKNGGKTWSRPRAIAKAHGA
TFNNPCPVYDAKTRTVTVVFQRYPAGVKERQPNIPDGWDD
EKCIRNFMIQSRNGGSSWTKPQEITKTTKRPSGVDIMASG
PNAGTQLKSGAHKGRLVIPMNEGPFGKWVISCIYSDDGGK
SWKLGQPTANMKGMVNETSIAETDNGGVVMVARHWGAGNC
RRIAWSQDGGETWGQVEDAPELFCDSTQNSLMTYSLSDQP
AYGGKSRILFSGPSAGRRIKGQVAMSYDNGKTWPVKKLLG
EGGFAYSSLAMVEPGIVGVLYEENQEHIKKLKFVPITMEW
LTDGEDTGLAPGKKAPVLK
A. muciniphila sialidase
SEQ ID NO: 24
MGLGLLCALGLSIPSVLGKESFEQARRGKFTTLSTKYGLM
SCRNGVAEIGGGGKSGEASLRMFGGQDAELKLDLKDTPSR
EVRLSAWAERWTGQAPFEFSIVAIGPNGEKKIYDGKDIRT
GGFHTRIEASVPAGTRSLVFRLTSPENKGMKLDDLFLVPC
IPMKVNPQVEMASSAYPVMVRIPCSPVLSLNVRTDGCLNP
QFLTAVNLDFTGTTKLSDIESVAVIRGEEAPIIHHGEEPF
PKDSSQVLGTVKLAGSARPQISVKGKMELEPGDNYLWACV
TMKEGASLDGRVVVRPASVVAGNKPVRVANAAPVAQRIGV
AVVRHGDFKSKFYRIPGLARSRKGTLLAVYDIRYNHSGDL
PANIDVGVSRSTDGGRTWSDVKIAIDDSKIDPSLGATRGV
GDPAILVDEKTGRIWVAAIWSHRHSIWGSKSGDNSPEACG
QLVLAYSDDDGLTWSSPINITEQTKNKDWRILFNGPGNGI
CMKDGTLVFAAQYWDGKGVPWSTIVYSKDRGKTWHCGTGV
NQQTTEAQVIELEDGSVMINARCNWGGSRIVGVTKDLGQT
WEKHPTNRTAQLKEPVCQGSLLAVDGVPGAGRVVLFSNPN
TTSGRSHMTLKASTNDAGSWPEDKWLLYDARKGWGYSCLA
PVDKNHVGVLYESQGALNFLKIPYKDVLNAKNAR
B. thetaiotaomicron sialidase
SEQ ID NO: 25
MKRNHYLFTLILLLGCSIFVKASDTVFVHQTQIPILIERQ
DNVLFYFRLDAKESRMMDEIVLDFGKSVNLSDVQAVKLYY
GGTEALQDKGKKRFAPVDYISSHRPGNTLAAIPSYSIKCA
EALQPSAKVVLKSHYKLFPGINFFWISLQMKPETSLFTKI
SSELQSVKIDGKEAICEERSPKDIIHRMAVGVRHAGDDGS
ASFRIPGLVTSNKGTLLGVYDVRYNSSVDLQEYVDVGLSR
STDGGKTWEKMRLPLSFGEYDGLPAAQNGVGDPSILVDTQ
TNTIWVVAAWTHGMGNQRAWWSSHPGMDLYQTAQLVMAKS
TDDGKTWSKPINITEQVKDPSWYFLLQGPGRGITMSDGTL
VFPTQFIDSTRVPNAGIMYSKDRGKTWKMHNMARTNTTEA
QVVETEPGVLMLNMRDNRGGSRAVAITKDLGKTWTEHPSS
RKALQEPVCMASLIHVEAEDNVLDKDILLFSNPNTTRGRN
HITIKASLDDGLTWLPEHQLMLDEGEGWGYSCLTMIDRET
IGILYESSAAHMTFQAVKLKDLIR
A. viscosus sialidase
SEQ ID NO: 26
MTSHSPFSRRHLPALLGSLPLAATGLIAAAPPAHAVPTSD
GLADVTITQVNAPADGLYSVGDVMTFNITLTNTSGEAHSY
APASTNLSGNVSKCRWRNVPAGTTKTDCTGLATHTVTAED
LKAGGFTPQIAYEVKAVEYAGKALSTPETIKGATSPVKAN
SLRVESITPSSSKEYYKLGDTVTYTVRVRSVSDKTINVAA
TESSFDDLGRQCHWGGLKPGKGAVYNCKPLTHTITQADVD
AGRWTPSITLTATGTDGTALQTLTATGNPINVVGDHPQAT
PAPAPDASTELPASMSQAQHVAPNTATDNYRIPAITTAPN
GDLLISYDERPKDNGNGGSDAPNPNHIVQRRSTDGGKTWS
APTYIHQGTETGKKVGYSDPSYVVDHQTGTIFNFHVKSYD
HGWGNSQAGTDPENRGIIQAEVSTSTDNGWTWTHRTITAD
ITKDNPWTARFAASGQGIQIQHGPHAGRLVQQYTIRTAGG
AVQAVSVYSDDHGKTWQAGTPVGTGMDENKVVELSDGSLM
LNSRASDSSGFRKVAHSTDGGQTWSEPVSDKNLPDSVDNA
QIIRAFPNAAPDDPRAKVLLLSHSPNPKPWSRDRGTISMS
CDDGASWTTSKVFHEPFVGYTTIAVQSDGSIGLLSEDAHD
GANYGGIWYRNFTMNWLGEQCGQKPAEPSPAPSPTAAPSA
APSEQPAPSAAPSTEPTOAPAPSSAPEPSAVPEPSSAPAP
EPTTAPSTEPTPTPAPSSAPEPSAGPTAAPAPETSSAPAA
EPTQAPTVAPSAEPTQVPGAQPSAAPSEKPGAQPSSAPKP
DATGRAPSVVNPKATAAPSGKASSSASPAPSRSATATSKP
GMEPDEIDRPSDGAMAQPTGGASAPSAAPTQAAKAGSRLS
RTGTNALLVLGLAGVAVVGGYLLLRARRSKN
DAS181 without initial Met and without
anchoring domain
SEQ ID NO: 27
GDHPQ
ATPAPAPDASTELPASMSQAQHLAANTATDNYRIPAITT
APNGDLLISYDERPKDNGNGGSDAPNPNHIVQRRSTDGGK
TWSAPTYIHQGTETGKKVGYSDPSYVVDHQTGTIFNFHVK
SYDQGWGGSRGGTDPENRGIIQAEVSTSTDNGWTWTHRTI
TADITKDKPWTARFAASGQGIQIQHGPHAGRLVQQYTIRT
AGGAVQAVSVYSDDHGKTWQAGTPIGTGMDENKVVELSDG
SLMLNSRASDGSGFRKVAHSTDGGQTWSEPVSDKNLPDSV
DNAQIIRAFPNAAPDDPRAKVLLLSHSPNPRPWSRDRGTI
SMSCDDGASWTTSKVFHEPFVGYTTIAVQSDGSIGLLSED
AHNGADYGGIWYRNFTMNWLGEQCGQKPA
Construct 1: mIg-K_DAS181 Protein sequence
SEQ ID NO: 28
METDTLLLWVLLLWVPGSTGDGDHPQATPAPAPDASTELP
ASMSQAQHLAANTATDNYRIPAITTAPNGDLLISYDERPK
DNGNGGSDAPNPNHIVQRRSTDGGKTWSAPTYIHQGTETG
KKVGYSDPSYVVDHQTGTIFNFHVKSYDQGWGGSRGGTDP
ENRGIIQAEVSTSTDNGWTWTHRTITADITKDKPWTARFA
ASGQGIQIQHGPHAGRLVQQYTIRTAGGAVQAVSVYSDDH
GKTWQAGTPIGTGMDENKVVELSDGSLMLNSRASDGSGFR
KVAHSTDGGQTWSEPVSDKNLPDSVDNAQIIRAFPNAAPD
DPRAKVLLLSHSPNPRPWSRDRGTISMSCDDGASWTTSKV
FHEPFVGYTTIAVQSDGSIGLLSEDAHNGADYGGIWYRNF
TMNWLGEQCGQKPAKRKKKGGKNGKNRRNRKKKNP
Construct 2: mIg-K_DAS185 Protein sequence
SEQ ID NO: 29
METDTLLLWVLLLWVPGSTGDGDHPQATPAPAPDASTELP
ASMSQAQHLAANTATDNYRIPAITTAPNGDLLISYDERPK
DNGNGGSDAPNPNHIVQRRSTDGGKTWSAPTYIHQGTETG
KKVGYSDPSYVVDHQTGTIFNFHVKSYDQGWGGSRGGTDP
ENRGIIQAEVSTSTDNGWTWTHRTITADITKDKPWTARFA
ASGQGIQIQHGPHAGRLVQQYTIRTAGGAVQAVSVYSDDH
GKTWQAGTPIGTGMDENKVVELSDGSLMLNSRASDGSGFR
KVAHSTDGGQTWSEPVSDKNLPDSVDNAQIIRAFPNAAPD
DPRAKVLLLSHSPNPRPWSRDRGTISMSCDDGASWTTSKV
FHEPFVGFTTIAVQSDGSIGLLSEDAHNGADYGGIWYRNF
TMNWLGEQCGQKPAKRKKKGGKNGKNRRNRKKKNP
Construct 3: mIg-K_Neu2-AR Protein sequence
SEQ ID NO: 30
METDTLLLWVLLLWVPGSTGDMASLPVLQKESVFQSGAHA
YRIPALLYLPGQQSLLAFAEQRASKKDEHAELIVLRRGDY
DAPTHQVQWQAQEVVAQARLDGHRSMNPCPLYDAQTGTLF
LFFIAIPGQVTEQQQLQTRANVTRLCQVTSTDHGRTWSSP
RDLTDAAIGPAYREWSTFAVGPGHCLQLHDRARSLVVPAY
AYRKLHPIQRPIPSAFCFLSHDHGRTWARGHFVAQDTLEC
QVAEVETGEQRVVTLNARSHLRARVQAQSTNDGLDFQESQ
LVKKLVEPPPQGCQGSVISFPSPRSGPGSPAQWLLYTHPT
HSWQRADLGAYLNPRPPAPEAWSEPVLLAKGSCAYSDLQS
MGTGPDGSPLFGCLYEANDYEEIVFLMFTLKQAFPAEYLP
QKRKKKGGKNGKNRRNRKKKNP
Construct 4: DAS181(-AR)_TM Protein Sequence
SEQ ID NO: 31
METDTLLLWVLLLWVPGSTGDYPYDVPDYAGATPARSPGM
GDHPQATPAPAPDASTELPASMSQAQHLAANTATDNYRIP
AITTAPNGDLLISYDERPKDNGNGGSDAPNPNHIVQRRST
DGGKTWSAPTYIHQGTETGKKVGYSDPSYVVDHQTGTIFN
FHVKSYDQGWGGSRGGTDPENRGIIQAEVSTSTDNGWTWT
HRTITADITKDKPWTARFAASGQGIQIQHGPHAGRLVQQY
TIRTAGGAVQAVSVYSDDHGKTWQAGTPIGTGMDENKVVE
LSDGSLMLNSRASDGSGFRKVAHSTDGGQTWSEPVSDKNL
PDSVDNAQIIRAFPNAAPDDPRAKVLLLSHSPNPRPWSRD
RGTISMSCDDGASWTTSKVFHEPFVGFTTIAVQSDGSIGL
LSEDAHNGADYGGIWYRNFTMNWLGEQCGQKPAVDEQKLI
SEEDLNAVGQDTQEVIVVPHSLPFKVVVISAILALVVLTI
ISLIILIMLWQKKPR
Construct 5:
DAS185(-AR)_TM Protein Sequence
SEQ ID NO: 32
METDTLLLWVLLLWVPGSTGDYPYDVPDYAGATPARSPGM
GDHPQATPAPAPDASTELPASMSQAQHLAANTATDNYRIP
AITTAPNGDLLISYDERPKDNGNGGSDAPNPNHIVQRRST
DGGKTWSAPTYIHQGTETGKKVGYSDPSYVVDHQTGTIFN
FHVKSYDQGWGGSRGGTDPENRGIIQAEVSTSTDNGWTWT
HRTITADITKDKPWTARFAASGQGIQIQHGPHAGRLVQQY
TIRTAGGAVQAVSVYSDDHGKTWQAGTPIGTGMDENKVVE
LSDGSLMLNSRASDGSGFRKVAHSTDGGQTWSEPVSDKNL
PDSVDNAQIIRAFPNAAPDDPRAKVLLLSHSPNPRPWSRD
RGTISMSCDDGASWTTSKVFHEPFVGFTTIAVQSDGSIGL
LSEDAHNGADYGGIWYRNFTMNWLGEQCGQKPAVDEQKLI
SEEDLNAVGQDTQEVIVVPHSLPFKVVVISAILALVVLTI
ISLIILIMLWQKKPR
Construct 6: Neu2 TM
SEQ ID NO: 33
METDTLLLWVLLLWVPGSTGDYPYDVPDYAGATPARSPGM
ASLPVLQKESVFQSGAHAYRIPALLYLPGQQSLLAFAEQR
ASKKDEHAELIVLRRGDYDAPTHQVQWQAQEVVAQARLDG
HRSMNPCPLYDAQTGTLFLFFIAIPGQVTEQQQLQTRANV
TRLCQVTSTDHGRTWSSPRDLTDAAIGPAYREWSTFAVGP
GHCLQLHDRARSLVVPAYAYRKLHPIQRPIPSAFCFLSHD
HGRTWARGHFVAQDTLECQVAEVETGEQRVVTLNARSHLR
ARVQAQSTNDGLDFQESQLVKKLVEPPPQGCQGSVISFPS
PRSGPGSPAQWLLYTHPTHSWQRADLGAYLNPRPPAPEAW
SEPVLLAKGSCAYSDLQSMGTGPDGSPLFGCLYEANDYEE
IVFLMFTLKQAFPAEYLPQVDEQKLISEEDLNAVGQDTQE
VIVVPHSLPFKVVVISAILALVVLTIISLIILIMLWQKKP
R
Construct 1:
mIg-K_DAS181 Nucleotide sequence
SEQ ID NO: 34
ATGgagacagacacactcctgctatgggtactgctgctct
gggttccaggttccactggtgacGGCGACCACCCACAGGC
AACACCAGCACCTGCCCCAGATGCCTCCACCGAGCTGCCA
GCAAGCATGTCCCAGGCACAGCACCTGGCAGCAAATACCG
CAACAGACAACTACAGAATCCCCGCCATCACCACAGCCCC
AAATGGCGATCTGCTGATCAGCTATGACGAGCGCCCCAAG
GATAACGGAAATGGAGGCTCCGACGCACCAAACCCTAATC
ACATCGTGCAGCGGAGATCTACCGATGGCGGCAAGACATG
GAGCGCCCCTACCTACATCCACCAGGGCACCGAGACAGGC
AAGAAGGTCGGCTACTCTGACCCAAGCTATGTGGTGGATC
ACCAGACCGGCACAATCTTCAACTTTCACGTGAAGTCCTA
TGACCAGGGATGGGGAGGCTCTAGGGGCGGCACCGATCCT
GAGAATCGCGGCATCATCCAGGCCGAGGTGTCTACCAGCA
CAGACAACGGCTGGACCTGGACACACCGGACCATCACAGC
CGACATCACAAAGGATAAGCCCTGGACCGCAAGATTCGCA
GCAAGCGGACAGGGCATCCAGATCCAGCACGGACCTCACG
CAGGCCGGCTGGTGCAGCAGTACACCATCAGAACAGCAGG
AGGAGCAGTGCAGGCCGTGTCCGTGTATTCTGACGATCAC
GGCAAGACCTGGCAGGCAGGCACCCCAATCGGCACAGGCA
TGGACGAGAATAAGGTGGTGGAGCTGAGCGATGGCTCCCT
GATGCTGAACTCTAGGGCCAGCGACGGCTCCGGCTTCCGC
AAGGTGGCACACTCTACAGACGGAGGACAGACCTGGTCCG
AGCCCGTGTCTGATAAGAATCTGCCTGACAGCGTGGATAA
CGCCCAGATCATCCGGGCCTTTCCTAATGCCGCCCCAGAC
GATCCCAGAGCCAAGGTGCTGCTGCTGTCCCACTCTCCAA
ACCCAAGGCCTTGGAGCCGGGACAGAGGCACAATCAGCAT
GTCCTGCGACGATGGCGCCAGCTGGACCACATCCAAGGTG
TTCCACGAGCCATTTGTGGGCTACACCACAATCGCCGTGC
AGTCTGATGGCAGCATCGGACTGCTGAGCGAGGACGCACA
CAATGGCGCCGATTACGGCGGCATCTGGTATCGGAACTTC
ACCATGAACTGGCTGGGCGAGCAGTGTGGCCAGAAGCCAG
CCAAGCGGAAGAAGAAGGGCGGCAAGAACGGCAAGAATAG
GCGCAACCGGAAGAAGAAGAACCCCTGATGA
Construct 2:
mIg-K_DAS185 Nucleotide sequence
SEQ ID NO: 35
ATGgagacagacacactcctgctatgggtactgctgctct
gggttccaggttccactggtgacGGCGACCACCCACAGGC
AACACCAGCACCTGCCCCAGATGCCTCCACCGAGCTGCCA
GCAAGCATGTCCCAGGCACAGCACCTGGCAGCAAATACCG
CAACAGACAACTACAGAATCCCCGCCATCACCACAGCCCC
AAATGGCGATCTGCTGATCAGCTATGACGAGCGCCCCAAG
GATAACGGAAATGGAGGCTCCGACGCACCAAACCCTAATC
ACATCGTGCAGCGGAGATCTACCGATGGCGGCAAGACATG
GAGCGCCCCTACCTACATCCACCAGGGCACCGAGACAGGC
AAGAAGGTCGGCTACTCTGACCCAAGCTATGTGGTGGATC
ACCAGACCGGCACAATCTTCAACTTTCACGTGAAGTCCTA
TGACCAGGGATGGGGAGGCTCTAGGGGCGGCACCGATCCT
GAGAATCGCGGCATCATCCAGGCCGAGGTGTCTACCAGCA
CAGACAACGGCTGGACCTGGACACACCGGACCATCACAGC
CGACATCACAAAGGATAAGCCCTGGACCGCAAGATTCGCA
GCAAGCGGACAGGGCATCCAGATCCAGCACGGACCTCACG
CAGGCCGGCTGGTGCAGCAGTACACCATCAGAACAGCAGG
AGGAGCAGTGCAGGCCGTGTCCGTGTATTCTGACGATCAC
GGCAAGACCTGGCAGGCAGGCACCCCAATCGGCACAGGCA
TGGACGAGAATAAGGTGGTGGAGCTGAGCGATGGCTCCCT
GATGCTGAACTCTAGGGCCAGCGACGGCTCCGGCTTCCGC
AAGGTGGCACACTCTACAGACGGAGGACAGACCTGGTCCG
AGCCCGTGTCTGATAAGAATCTGCCTGACAGCGTGGATAA
CGCCCAGATCATCCGGGCCTTTCCTAATGCCGCCCCAGAC
GATCCCAGAGCCAAGGTGCTGCTGCTGTCCCACTCTCCAA
ACCCAAGGCCTTGGAGCCGGGACAGAGGCACAATCAGCAT
GTCCTGCGACGATGGCGCCAGCTGGACCACATCCAAGGTG
TTCCACGAGCCATTTGTGGGCTTCACCACAATCGCCGTGC
AGTCTGATGGCAGCATCGGACTGCTGAGCGAGGACGCACA
CAATGGCGCCGATTACGGCGGCATCTGGTATCGGAACTTC
ACCATGAACTGGCTGGGCGAGCAGTGTGGCCAGAAGCCAG
CCAAGCGGAAGAAGAAGGGCGGCAAGAACGGCAAGAATAG
GCGCAACCGGAAGAAGAAGAACCCCTGATGA
Construct 3:
mIg-K_Neu2-AR Nucleotide Sequence
SEQ ID NO: 36
ATGgagacagacacactcctgctatgggtactgctgctct
gggttccaggttccactggtgacATGGCCAGCCTGCCTGT
GCTGCAGAAGGAGAGCGTGTTCCAGTCCGGCGCCCACGCA
TACAGAATCCCCGCCCTGCTGTATCTGCCTGGCCAGCAGT
CCCTGCTGGCCTTTGCCGAGCAGAGAGCCTCTAAGAAGGA
CGAGCACGCAGAGCTGATCGTGCTGAGGAGGGGCGACTAC
GATGCACCAACCCACCAGGTGCAGTGGCAGGCACAGGAGG
TGGTGGCACAGGCAAGGCTGGACGGACACCGCAGCATGAA
TCCATGCCCCCTGTATGATGCCCAGACCGGCACACTGTTC
CTGTTCTTTATCGCAATCCCCGGCCAGGTGACCGAGCAGC
AGCAGCTGCAGACCAGAGCCAACGTGACAAGACTGTGCCA
GGTGACCTCCACAGACCACGGCAGGACCTGGAGCAGCCCT
CGCGACCTGACAGATGCAGCAATCGGACCAGCATACAGGG
AGTGGTCTACATTCGCCGTGGGCCCTGGCCACTGCCTGCA
GCTGCACGATCGGGCCAGAAGCCTGGTGGTGCCAGCCTAC
GCCTATCGGAAGCTGCACCCCATCCAGAGACCTATCCCAT
CTGCCTTCTGCTTTCTGAGCCACGACCACGGCAGAACTTG
GGCCAGAGGCCACTTTGTGGCCCAGGATACACTGGAGTGT
CAGGTGGCAGAGGTGGAGACCGGAGAGCAGAGGGTGGTGA
CACTGAATGCACGCAGCCACCTGAGGGCCCGCGTGCAGGC
CCAGTCCACCAACGACGGCCTGGATTTCCAGGAGTCTCAG
CTGGTGAAGAAGCTGGTGGAGCCACCTCCACAGGGATGTC
AGGGCTCTGTGATCAGCTTTCCCTCCCCTCGGTCTGGCCC
AGGCAGCCCAGCACAGTGGCTGCTGTACACCCACCCCACA
CACTCCTGGCAGAGGGCAGACCTGGGAGCATATCTGAATC
CAAGACCCCCTGCACCAGAGGCCTGGTCCGAGCCTGTGCT
GCTGGCCAAGGGCTCTTGCGCCTACAGCGACCTGCAGAGC
ATGGGCACCGGACCTGATGGCTCTCCACTGTTCGGCTGTC
TGTACGAGGCCAACGATTATGAGGAGATCGTGTTCCTGAT
GTTTACACTGAAGCAGGCCTTTCCTGCCGAGTATCTGCCA
CAGAAGCGGAAGAAGAAGGGCGGCAAGAACGGCAAGAATC
GGAGAAACCGGAAGAAGAAGAACCCTTGATGA
Construct 4:
DAS181(-AR)_TM Nucleotide sequence
SEQ ID NO: 37
atggagacagacacactcctgctatgggtactgctgctct
gggttccaggttccactggtgacTATCCATATGATGTTCC
AGATTATGCTGGGGCCACGCCGGCCAGATCTCCCGGGATG
GGCGACCACCCACAGGCAACACCAGCACCTGCCCCAGATG
CCTCCACCGAGCTGCCAGCAAGCATGTCCCAGGCACAGCA
CCTGGCAGCAAATACCGCAACAGACAACTACAGAATCCCC
GCCATCACCACAGCCCCAAATGGCGATCTGCTGATCAGCT
ATGACGAGCGCCCCAAGGATAACGGAAATGGAGGCTCCGA
CGCACCAAACCCTAATCACATCGTGCAGCGGAGATCTACC
GATGGCGGCAAGACATGGAGCGCCCCTACCTACATCCACC
AGGGCACCGAGACAGGCAAGAAGGTCGGCTACTCTGACCC
AAGCTATGTGGTGGATCACCAGACCGGCACAATCTTCAAC
TTTCACGTGAAGTCCTATGACCAGGGATGGGGAGGCTCTA
GGGGCGGCACCGATCCTGAGAATCGCGGCATCATCCAGGC
CGAGGTGTCTACCAGCACAGACAACGGCTGGACCTGGACA
CACCGGACCATCACAGCCGACATCACAAAGGATAAGCCCT
GGACCGCAAGATTCGCAGCAAGCGGACAGGGCATCCAGAT
CCAGCACGGACCTCACGCAGGCCGGCTGGTGCAGCAGTAC
ACCATCAGAACAGCAGGAGGAGCAGTGCAGGCCGTGTCCG
TGTATTCTGACGATCACGGCAAGACCTGGCAGGCAGGCAC
CCCAATCGGCACAGGCATGGACGAGAATAAGGTGGTGGAG
CTGAGCGATGGCTCCCTGATGCTGAACTCTAGGGCCAGCG
ACGGCTCCGGCTTCCGCAAGGTGGCACACTCTACAGACGG
AGGACAGACCTGGTCCGAGCCCGTGTCTGATAAGAATCTG
CCTGACAGCGTGGATAACGCCCAGATCATCCGGGCCTTTC
CTAATGCCGCCCCAGACGATCCCAGAGCCAAGGTGCTGCT
GCTGTCCCACTCTCCAAACCCAAGGCCTTGGAGCCGGGAC
AGAGGCACAATCAGCATGTCCTGCGACGATGGCGCCAGCT
GGACCACATCCAAGGTGTTCCACGAGCCATTTGTGGGCTA
CACCACAATCGCCGTGCAGTCTGATGGCAGCATCGGACTG
CTGAGCGAGGACGCACACAATGGCGCCGATTACGGCGGCA
TCTGGTATCGGAACTTCACCATGAACTGGCTGGGCGAGCA
GTGTGGCCAGAAGCCAGCCGTCGACGAACAAAAACTCATC
TCAGAAGAGGATCTGaatgctgtgggccaggacacgcagg
aggtcatcgtggtgccacactccttgccctttaaggtggt
ggtgatctcagccatcctggccctggtggtgctcaccatc
atctcccttatcatcctcatcatgctttggcagaagaagc
cacgt
Construct 5:
DAS185(-AR)_TM Nucleotide sequence
SEQ ID NO: 38
atggagacagacacactcctgctatgggtactgctgctct
gggttccaggttccactggtgacTATCCATATGATGTTCC
AGATTATGCTGGGGCCACGCCGGCCAGATCTCCCGGGATG
GGCGACCACCCACAGGCAACACCAGCACCTGCCCCAGATG
CCTCCACCGAGCTGCCAGCAAGCATGTCCCAGGCACAGCA
CCTGGCAGCAAATACCGCAACAGACAACTACAGAATCCCC
GCCATCACCACAGCCCCAAATGGCGATCTGCTGATCAGCT
ATGACGAGCGCCCCAAGGATAACGGAAATGGAGGCTCCGA
CGCACCAAACCCTAATCACATCGTGCAGCGGAGATCTACC
GATGGCGGCAAGACATGGAGCGCCCCTACCTACATCCACC
AGGGCACCGAGACAGGCAAGAAGGTCGGCTACTCTGACCC
AAGCTATGTGGTGGATCACCAGACCGGCACAATCTTCAAC
TTTCACGTGAAGTCCTATGACCAGGGATGGGGAGGCTCTA
GGGGCGGCACCGATCCTGAGAATCGCGGCATCATCCAGGC
CGAGGTGTCTACCAGCACAGACAACGGCTGGACCTGGACA
CACCGGACCATCACAGCCGACATCACAAAGGATAAGCCCT
GGACCGCAAGATTCGCAGCAAGCGGACAGGGCATCCAGAT
CCAGCACGGACCTCACGCAGGCCGGCTGGTGCAGCAGTAC
ACCATCAGAACAGCAGGAGGAGCAGTGCAGGCCGTGTCCG
TGTATTCTGACGATCACGGCAAGACCTGGCAGGCAGGCAC
CCCAATCGGCACAGGCATGGACGAGAATAAGGTGGTGGAG
CTGAGCGATGGCTCCCTGATGCTGAACTCTAGGGCCAGCG
ACGGCTCCGGCTTCCGCAAGGTGGCACACTCTACAGACGG
AGGACAGACCTGGTCCGAGCCCGTGTCTGATAAGAATCTG
CCTGACAGCGTGGATAACGCCCAGATCATCCGGGCCTTTC
CTAATGCCGCCCCAGACGATCCCAGAGCCAAGGTGCTGCT
GCTGTCCCACTCTCCAAACCCAAGGCCTTGGAGCCGGGAC
AGAGGCACAATCAGCATGTCCTGCGACGATGGCGCCAGCT
GGACCACATCCAAGGTGTTCCACGAGCCATTTGTGGGCTT
CACCACAATCGCCGTGCAGTCTGATGGCAGCATCGGACTG
CTGAGCGAGGACGCACACAATGGCGCCGATTACGGCGGCA
TCTGGTATCGGAACTTCACCATGAACTGGCTGGGCGAGCA
GTGTGGCCAGAAGCCAGCCGTCGACGAACAAAAACTCATC
TCAGAAGAGGATCTGaatgctgtgggccaggacacgcagg
aggtcatcgtggtgccacactccttgccctttaaggtggt
ggtgatctcagccatcctggccctggtggtgctcaccatc
atctcccttatcatcctcatcatgctttggcagaagaagc
cacgt
Construct 6: Neu2_TM Nucleotide sequence
SEQ ID NO: 39
atggagacagacacactcctgctatgggtactgctgctct
gggttccaggttccactggtgacTATCCATATGATGTTCC
AGATTATGCTGGGGCCACGCCGGCCAGATCTCCCGGGATG
GCCAGCCTGCCTGTGCTGCAGAAGGAGAGCGTGTTCCAGT
CCGGCGCCCACGCATACAGAATCCCCGCCCTGCTGTATCT
GCCTGGCCAGCAGTCCCTGCTGGCCTTTGCCGAGCAGAGA
GCCTCTAAGAAGGACGAGCACGCAGAGCTGATCGTGCTGA
GGAGGGGCGACTACGATGCACCAACCCACCAGGTGCAGTG
GCAGGCACAGGAGGTGGTGGCACAGGCAAGGCTGGACGGA
CACCGCAGCATGAATCCATGCCCCCTGTATGATGCCCAGA
CCGGCACACTGTTCCTGTTCTTTATCGCAATCCCCGGCCA
GGTGACCGAGCAGCAGCAGCTGCAGACCAGAGCCAACGTG
ACAAGACTGTGCCAGGTGACCTCCACAGACCACGGCAGGA
CCTGGAGCAGCCCTCGCGACCTGACAGATGCAGCAATCGG
ACCAGCATACAGGGAGTGGTCTACATTCGCCGTGGGCCCT
GGCCACTGCCTGCAGCTGCACGATCGGGCCAGAAGCCTGG
TGGTGCCAGCCTACGCCTATCGGAAGCTGCACCCCATCCA
GAGACCTATCCCATCTGCCTTCTGCTTTCTGAGCCACGAC
CACGGCAGAACTTGGGCCAGAGGCCACTTTGTGGCCCAGG
ATACACTGGAGTGTCAGGTGGCAGAGGTGGAGACCGGAGA
GCAGAGGGTGGTGACACTGAATGCACGCAGCCACCTGAGG
GCCCGCGTGCAGGCCCAGTCCACCAACGACGGCCTGGATT
TCCAGGAGTCTCAGCTGGTGAAGAAGCTGGTGGAGCCACC
TCCACAGGGATGTCAGGGCTCTGTGATCAGCTTTCCCTCC
CCTCGGTCTGGCCCAGGCAGCCCAGCACAGTGGCTGCTGT
ACACCCACCCCACACACTCCTGGCAGAGGGCAGACCTGGG
AGCATATCTGAATCCAAGACCCCCTGCACCAGAGGCCTGG
TCCGAGCCTGTGCTGCTGGCCAAGGGCTCTTGCGCCTACA
GCGACCTGCAGAGCATGGGCACCGGACCTGATGGCTCTCC
ACTGTTCGGCTGTCTGTACGAGGCCAACGATTATGAGGAG
ATCGTGTTCCTGATGTTTACACTGAAGCAGGCCTTTCCTG
CCGAGTATCTGCCACAGGTCGACGAACAAAAACTCATCTC
AGAAGAGGATCTGaatgctgtgggccaggacacgcaggag
gtcatcgtggtgccacactccttgccctttaaggtggtgg
tgatctcagccatcctggccctggtggtgctcaccatcat
ctcccttatcatcctcatcatgctttggcagaagaagcca
cgt
Exemplary amino acid secretion sequence
SEQ ID NO: 40
METDTLLLWVLLLWVPGSTGD
HA tag amino acid sequence
SEQ ID NO: 41
YPYDVPDYA
N-terminal cloning site amino acid sequence
SEQ ID NO: 42
GATPARSPG
C-terminal cloning site amino acid sequence
SEQ ID NO: 43
VD
Myc Tag amino acid sequence
SEQ ID NO: 44
EQKLISEEDL
human PDGFR TM2 domain
SEQ ID NO: 45
NAVGQDTQEVIVVPHSLPFKVVVISAILALVVLTIISLII
LIMLWQKKPR
TM domain (NM_006139)
SEQ ID NO: 46
FWVLVVVGGVLACYSLLVTVAFIIFWV
human CD4 TM domain (M35160)
SEQ ID NO: 47
MALIVLGGVAGLLLFIGLGIFF
human CD8 TM1 domain (NM_001768)
SEQ ID NO: 48
IYIWAPLAGTCGVLLLSLVIT
human CD8 TM2 domain (NM_001768)
SEQ ID NO: 49
IYIWAPLAGTCGVLLLSLVITLY
human CD8 TM3 domain (NM_001768)
SEQ ID NO: 50
IYIWAPLAGTCGVLLLSLVITLYC
human 41BB TM domain (NM_001561)
SEQ ID NO: 51
IISFFLALTSTALLFLLFFLTLRFSVV
human PDGFR TM1 domain
SEQ ID NO: 52
VVISAILALVVLTIISLIILI
Salmonella typhimurium sialidase
SEQ ID NO: 53
TVEKSVVFKAEGEHFTDQKGNTIVGSGSGGTTKYFRIPAM
CTTSKGTIVVFADARHNTASDQSFIDTAAARSTDGGKTWN
KKIAIYNDRVNSKLSRVMDPTCIVANIQGRETILVMVGKW
NNNDKTWGAYRDKAPDTDWDLVLYKSTDDGVTFSKVETNI
HDIVTKNGTISAMLGGVGSGLQLNDGKLVFPVQMVRTKNI
TTVLNTSFIYSTDGITWSLPSGYCEGFGSENNIIEFNASL
VNNIRNSGLRRSFETKDFGKTWTEFPPMDKKVDNRNHGVQ
GSTITIPSGNKLVAAHSSAQNKNNDYTRSDISLYAHNLYS
GEVKLIDDFYPKVGNASGAGYSCLSYRKNVDKETLYVVYE
ANGSIEFQDLSRHLPVIKSYN
Vibrio cholera sialidase
SEQ ID NO: 54
MRFKNVKKTALMLAMFGMATSSNAALFDYNATGDTEFDSP
AKQGWMQDNTNNGSGVLTNADGMPAWLVQGIGGRAQWTYS
LSTNQHAQASSFGWRMTTEMKVLSGGMITNYYANGTQRVL
PIISLDSSGNLVVEFEGQTGRTVLATGTAATEYHKFELVF
LPGSNPSASFYFDGKLIRDNIQPTASKQNMIVWGNGSSNT
DGVAAYRDIKFEIQGDVIFRGPDRIPSIVASSVTPGVVTA
FAEKRVGGGDPGALSNTNDIITRTSRDGGITWDTELNLTE
QINVSDEFDFSDPRPIYDPSSNTVLVSYARWPTDAAQNGD
RIKPWMPNGIFYSVYDVASGNWQAPIDVTDQVKERSFQIA
GWGGSELYRRNTSLNSQQDWQSNAKIRIVDGAANQIQVAD
GSRKYVVTLSIDESGGLVANLNGVSAPIILQSEHAKVHSF
HDYELQYSALNHTTTLFVDGQQITTWAGEVSQENNIQFGN
ADAQIDGRLHVQKIVLTQQGHNLVEFDAFYLAQQTPEVEK
DLEKLGWTKIKTGNTMSLYGNASVNPGPGHGITLTRQQNI
SGSQNGRLIYPAIVLDRFFLNVMSIYSDDGGSNWQTGSTL
PIPFRWKSSSILETLEPSEADMVELQNGDLLLTARLDFNQ
IVNGVNYSPRQQFLSKDGGITWSLLEANNANVFSNISTGT
VDASITRFEQSDGSHFLLFTNPQGNPAGTNGRQNLGLWFS
FDEGVTWKGPIQLVNGASAYSDIYQLDSENAIVIVETDNS
NMRILRMPITLLKQKLTLSQN
Lv-CD19-CAR Plasmid DNA sequence
SEQ ID NO: 55
ATGGAGTTTGGACTGAGCTGGCTGTTTCTCGTGGCCATTC
TGAAGGGCGTCCAGTGCAGCAGAGACATCCAGATGACCCA
GACAACCAGCTCTCTGAGCGCTAGCCTCGGAGATAGAGTG
ACCATTAGCTGTAGAGCCTCCCAAGACATTTCCAAGTACC
TCAACTGGTACCAGCAGAAGCCCGACGGCACCGTGAAGCT
GCTGATCTACCACACCAGCAGACTGCACTCCGGAGTGCCC
TCTAGGTTTTCCGGATCCGGCAGCGGCACAGACTACTCTC
TGACCATCTCCAATCTGGAGCAAGAGGACATCGCCACCTA
CTTCTGCCAGCAAGGCAACACACTGCCTTACACATTCGGC
GGCGGAACAAAGCTCGAACTGAAAAGAGGCGGCGGCGGAA
GCGGAGGAGGAGGATCCGGAGGCGGAGGATCCGGCGGAGG
AGGCTCCGAAGTCCAGCTGCAACAAAGCGGACCCGGACTG
GTGGCTCCCAGCCAATCTCTGAGCGTGACATGCACAGTGT
CCGGCGTCTCTCTGCCCGACTACGGAGTCAGCTGGATTAG
ACAGCCTCCTAGAAAGGGACTGGAGTGGCTGGGAGTCATC
TGGGGCAGCGAGACCACCTACTATAACTCCGCCCTCAAGT
CTAGGCTCACCATCATCAAAGACAACAGCAAGAGCCAAGT
GTTCCTCAAGATGAACAGCCTCCAGACCGACGACACCGCC
ATCTACTACTGCGCCAAACACTACTACTACGGAGGCAGCT
ACGCTATGGATTACTGGGGCCAAGGCACCACAGTCACAGT
GAGCAGCTATGTGACCGTGAGCAGCCAAGACCCCGCCAAA
GATCCCAAGTTCTGGGTGCTGGTCGTGGTGGGAGGCGTGC
TGGCTTGTTATTCTCTGCTGGTGACCGTGGCCTTCATCAT
CTTCTGGGTGAGGAGCAAGAGATCCAGACTGCTGCACAGC
GACTACATGAACATGACACCTAGAAGGCCCGGCCCCACAA
GGAAACATTACCAGCCCTACGCCCCCCCTAGAGACTTCGC
TGCCTATAGATCCAAGAGAGGAAGAAAAAAGCTGCTCTAC
ATCTTCAAGCAGCCCTTCATGAGGCCCGTGCAAACAACAC
AAGAGGAGGACGGATGTAGCTGTAGATTCCCCGAGGAGGA
AGAGGGAGGATGCGAGCTGAGAGTGAAGTTCTCTAGGAGC
GCCGATGCTCCCGCTTATCAGCAAGGCCAGAACCAGCTGT
ACAATGAGCTGAATCTGGGAAGAAGGGAAGAATACGACGT
GCTGGATAAGAGGAGGGGAAGAGACCCCGAGATGGGAGGC
AAGCCTAGAAGGAAGAACCCCCAAGAGGGACTGTACAACG
AGCTCCAAAAGGACAAGATGGCTGAAGCCTACAGCGAGAT
CGGAATGAAGGGAGAGAGAAGGAGGGGCAAGGGCCACGAT
GGACTCTACCAAGGCCTCAGCACAGCCACCAAGGACACCT
ACGACGCTCTGCACATGCAAGCTCTGCCCCCAGATGATGA
Lv-CD19-CAR Translated amino acid sequence
SEQ ID NO: 56
MEFGLSWLFLVAILKGVQCSRDIQMTQTTSSLSASLGDRV
TISCRASQDISKYLNWYQQKPDGTVKLLIYHTSRLHSGVP
SRFSGSGSGTDYSLTISNLEQEDIATYFCQQGNTLPYTFG
GGTKLELKRGGGGSGGGGSGGGGSGGGGSEVQLQQSGPGL
VAPSQSLSVTCTVSGVSLPDYGVSWIRQPPRKGLEWLGVI
WGSETTYYNSALKSRLTIIKDNSKSQVFLKMNSLQTDDTA
IYYCAKHYYYGGSYAMDYWGQGTTVTVSSYVTVSSQDPAK
DPKFWVLVVVGGVLACYSLLVTVAFIIFWVRSKRSRLLHS
DYMNMTPRRPGPTRKHYQPYAPPRDFAAYRSKRGRKKLLY
IFKQPFMRPVQTTQEEDGCSCRFPEEEEGGCELRVKFSRS
ADAPAYQQGQNQLYNELNLGRREEYDVLDKRRGRDPEMGG
KPRRKNPQEGLYNELQKDKMAEAYSEIGMKGERRRGKGHD
GLYQGLSTATKDTYDALHMQALPPDD
CD19-scFv amino acid sequence
SEQ ID NO: 57
MEFGLSWLFLVAILKGVQCSRDIQMTQTTSSLSASLGDRV
TISCRASQDISKYLNWYQQKPDGTVKLLIYHTSRLHSGVP
SRFSGSGSGTDYSLTISNLEQEDIATYFCQQGNTLPYTFG
GGTKLELKRGGGGSGGGGSGGGGSGGGGSEVQLQQSGPGL
VAPSQSLSVTCTVSGVSLPDYGVSWIRQPPRKGLEWLGVI
WGSETTYYNSALKSRLTIIKDNSKSQVFLKMNSLQTDDTA
IYYCAKHYYYGGSYAMDYWGQGTTVTVS
4-1BB costimulatory domain amino
acid sequence
SEQ ID NO: 58
KRGRKKLLYIFKQPFMRPVQTTQEEDGCSCRFPEEEEGGC
E
CD3 zeta chain amino acid sequence
SEQ ID NO: 59
LRVKFSRSADAPAYQQGQNQLYNELNLGRREEYDVLDKRRG
RDPEMGGKPRRKNPQEGLYNELQKDKMAEAYSEIGMKGER
RRGKGHDGLYQGLSTATKDTYDALHMQALPPDD
Lv-TM-Sial plasmid DNA sequence
SEQ ID NO: 60
ATGGAGTTTGGACTGAGCTGGCTGTTTCTGGTCGCCATTC
TGAAGGGCGTGCAGTGCGGAGACCACCCTCAAGCTACACC
CGCCCCCGCCCCCGATGCTAGCACCGAGCTCCCCGCCAGC
ATGAGCCAAGCCCAACATCTGGCCGCTAACACCGCCACCG
ACAACTACAGAATCCCCGCCATCACCACCGCTCCCAATGG
AGATCTGCTGATCAGCTATGACGAGAGGCCCAAGGATAAC
GGAAACGGAGGCAGCGACGCTCCCAACCCTAACCACATCG
TCCAGAGAAGGTCCACAGATGGCGGAAAGACATGGTCCGC
TCCCACCTACATCCACCAAGGCACAGAGACCGGAAAGAAG
GTCGGCTACTCCGACCCCAGCTATGTCGTGGATCACCAGA
CCGGCACCATCTTCAACTTCCACGTGAAGAGCTACGACCA
AGGCTGGGGAGGATCCAGAGGCGGCACAGACCCCGAGAAT
AGAGGAATTATCCAAGCCGAGGTCTCCACCAGCACCGACA
ATGGCTGGACATGGACACATAGAACCATTACCGCCGACAT
TACCAAAGACAAGCCTTGGACAGCCAGATTCGCCGCTAGC
GGCCAAGGCATCCAGATCCAGCACGGACCTCACGCTGGCA
GACTGGTGCAGCAGTACACCATTAGAACAGCCGGAGGAGC
TGTGCAAGCCGTCTCCGTGTATTCCGACGACCATGGCAAG
ACATGGCAAGCCGGCACCCCCATTGGCACCGGCATGGACG
AGAACAAGGTGGTGGAGCTGTCCGACGGCTCTCTGATGCT
CAACTCTAGGGCTTCCGATGGCAGCGGATTCAGAAAGGTG
GCCCACAGCACCGATGGCGGACAGACATGGAGCGAGCCCG
TGAGCGACAAGAATCTGCCCGACTCCGTGGATAACGCCCA
GATCATCAGAGCCTTCCCTAACGCTGCCCCCGACGATCCT
AGAGCTAAAGTGCTGCTGCTGTCCCACAGCCCTAACCCTA
GACCTTGGTCCAGAGATAGGGGCACAATCAGCATGAGCTG
CGACGATGGCGCCAGCTGGACAACCAGCAAAGTGTTTCAC
GAGCCCTTCGTCGGCTACACAACCATCGCTGTCCAATCCG
ACGGATCCATCGGCCTCCTCAGCGAAGACGCCCACAATGG
AGCTGACTACGGCGGAATTTGGTATAGAAACTTCACCATG
AATTGGCTCGGCGAACAGTGCGGACAGAAGCCCGCCTCCT
ATGTGACAGTCAGCTCCCAAGACCCCGCCAAGGACCCCAA
GTTCTGGGTGCTGGTGGTCGTGGGAGGAGTGCTGGCTTGC
TATTCTCTGCTCGTCACCGTGGCCTTCATCATCTTCTGGG
TGAGGTCCAAGAGGAGCAGACTGCTGCACAGCGACTACAT
GAACATGACACCTAGAAGGCCCGGCCCCACAAGGAAACAC
TACCAACCCTACGCCCCCCCTAGAGATTTCGCCGCCTATA
GGAGCAAGAGGGGAAGGAAGAAGCTGCTGTACATTTTCAA
GCAGCCCTTCATGAGGCCCGTCCAAACCACACAAGAGGAG
GACGGATGTAGCTGTAGATTCCCCGAAGAGGAAGAGGGAG
GATGCGAACTGAGAGTGAAATTCTCTAGGAGCGCTGATGC
CCCCGCCTACCAGCAAGGCCAGAATCAGCTCTACAACGAG
CTCAATCTGGGCAGAAGAGAGGAGTACGACGTGCTGGATA
AGAGAAGGGGAAGGGACCCCGAGATGGGAGGCAAGCCCAG
AAGAAAGAACCCCCAAGAGGGACTGTACAATGAGCTCCAG
AAGGACAAGATGGCCGAAGCCTACTCCGAAATCGGCATGA
AGGGCGAAAGAAGGAGGGGCAAAGGACACGACGGACTGTA
TCAAGGCCTCTCCACCGCCACCAAAGACACCTACGATGCT
CTGCACATGCAAGCTCTCCCTCCTAGATGATGA
Lv-TM-Sial Translated amino acid sequence
SEQ ID NO: 61
MEFGLSWLFLVAILKGVQCGDHPQATPAPAPDASTELPAS
MSQAQHLAANTATDNYRIPAITTAPNGDLLISYDERPKDN
GNGGSDAPNPNHIVQRRSTDGGKTWSAPTYIHQGTETGKK
VGYSDPSYVVDHQTGTIFNFHVKSYDQGWGGSRGGTDPEN
RGIIQAEVSTSTDNGWTWTHRTITADITKDKPWTARFAAS
GQGIQIQHGPHAGRLVQQYTIRTAGGAVQAVSVYSDDHGK
TWQAGTPIGTGMDENKVVELSDGSLMLNSRASDGSGFRKV
AHSTDGGQTWSEPVSDKNLPDSVDNAQIIRAFPNAAPDDP
RAKVLLLSHSPNPRPWSRDRGTISMSCDDGASWTTSKVFH
EPFVGYTTIAVQSDGSIGLLSEDAHNGADYGGIWYRNFTM
NWLGEQCGQKPASYVTVSSQDPAKDPKFWVLVVVGGVLAC
YSLLVTVAFIIFWVRSKRSRLLHSDYMNMTPRRPGPTRKH
YQPYAPPRDFAAYRSKRGRKKLLYIFKQPFMRPVQTTQEE
DGCSCRFPEEEEGGCELRVKFSRSADAPAYQQGQNQLYNE
LNLGRREEYDVLDKRRGRDPEMGGKPRRKNPQEGLYNELQ
KDKMAEAYSEIGMKGERRRGKGHDGLYQGLSTATKDTYDA
LHMQALPPR
Hinge amino acid sequence
SEQ ID NO: 62
SYVTVSSQDPAKDPK
human CD28 TM domain
SEQ ID NO: 63
FWVLVVVGGVLACYSLLVTVAFIIFWVRSKRSRLLHSDYM
NMTPRRPGPTRKHYQPYAPPRDFAAYRS
Lv-SP-Sial plasmid DNA sequence
SEQ ID NO: 64
ATGGAGTTTGGACTCAGCTGGCTGTTCCTCGTGGCCATTC
TGAAGGGCGTCCAGTGCGGCGATCACCCTCAAGCTACCCC
CGCCCCCGCCCCCGACGCCTCCACAGAGCTCCCCGCCAGC
ATGTCCCAAGCCCAGCACCTCGCCGCCAATACAGCTACCG
ACAACTATAGAATCCCCGCTATCACAACAGCCCCCAATGG
AGATCTGCTGATCTCCTACGACGAGAGACCCAAGGATAAC
GGAAACGGAGGAAGCGACGCCCCCAACCCCAACCACATCG
TGCAGAGAAGAAGCACCGACGGCGGAAAGACATGGTCCGC
CCCTACCTACATCCACCAAGGCACAGAAACCGGCAAGAAG
GTGGGCTACAGCGACCCCTCCTACGTGGTGGACCACCAGA
CCGGCACCATCTTCAACTTTCACGTGAAGTCCTACGACCA
AGGCTGGGGAGGCTCCAGAGGCGGAACAGACCCCGAGAAT
AGGGGCATTATCCAAGCCGAGGTGTCCACAAGCACAGACA
ACGGATGGACATGGACCCATAGAACCATCACAGCCGACAT
CACCAAGGATAAGCCTTGGACCGCTAGATTTGCCGCTAGC
GGACAAGGCATCCAGATCCAGCACGGCCCCCACGCTGGAA
GACTGGTGCAGCAATACACCATCAGAACCGCTGGAGGCGC
CGTGCAAGCTGTGAGCGTCTACAGCGATGACCACGGCAAG
ACATGGCAAGCCGGAACCCCCATTGGCACCGGCATGGACG
AAAACAAGGTGGTGGAGCTGAGCGACGGATCTCTGATGCT
GAATAGCAGAGCCTCCGATGGCAGCGGATTCAGAAAGGTG
GCCCACTCCACCGATGGCGGACAGACATGGTCCGAACCCG
TGTCCGATAAGAATCTGCCCGACTCCGTGGACAACGCCCA
GATCATTAGAGCCTTCCCTAATGCCGCTCCCGACGACCCC
AGAGCCAAGGTGCTGCTGCTGAGCCACAGCCCTAACCCTA
GGCCTTGGAGCAGAGATAGAGGCACCATCAGCATGAGCTG
CGATGACGGCGCTAGCTGGACCACATCCAAAGTGTTCCAC
GAGCCTTTCGTGGGCTATACCACCATCGCCGTGCAGTCCG
ACGGCTCCATTGGACTGCTCAGCGAGGATGCCCATAATGG
CGCCGACTACGGCGGAATCTGGTATAGAAACTTCACCATG
AACTGGCTGGGCGAACAGTGTGGCCAGAAGCCCGCCAAGA
GGAAGAAGAAGGGCGGCAAGAACGGCAAGAATAGAAGGAA
TAGGAAAAAGAAAAATCCTTGATGA
Lv-SP-Sial Translated amino acid sequence
SEQ ID NO: 65
MEFGLSWLFLVAILKGVQCGDHPQATPAPAPDASTELPAS
MSQAQHLAANTATDNYRIPAITTAPNGDLLISYDERPKDN
GNGGSDAPNPNHIVQRRSTDGGKTWSAPTYIHQGTETGKK
VGYSDPSYVVDHQTGTIFNFHVKSYDQGWGGSRGGTDPEN
RGIIQAEVSTSTDNGWTWTHRTITADITKDKPWTARFAAS
GQGIQIQHGPHAGRLVQQYTIRTAGGAVQAVSVYSDDHGK
TWQAGTPIGTGMDENKVVELSDGSLMLNSRASDGSGFRKV
AHSTDGGQTWSEPVSDKNLPDSVDNAQIIRAFPNAAPDDP
RAKVLLLSHSPNPRPWSRDRGTISMSCDDGASWTTSKVFH
EPFVGYTTIAVQSDGSIGLLSEDAHNGADYGGIWYRNFTM
NWLGEQCGQKPAKRKKKGGKNGKNRRNRKKKNP

What is claimed is:

1. An engineered immune cell comprising a first heterologous nucleotide sequence encoding a sialidase and a second heterologous nucleotide sequence encoding a chimeric immune receptor.

2. The engineered immune cell of claim 1, wherein the sialidase is a human sialidase.

3. The engineered immune cell of claim 2, wherein the sialidase is selected from the group consisting of: NEU1, NEU2, NEU3, NEU4 and derivatives thereof.

4. The engineered immune cell of claim 1, wherein the sialidase is a Neu5Ac alpha(2,6)-Gal sialidase or a Neu5Ac alpha(2,3)-Gal sialidase.

5. The engineered immune cell of claim 1, wherein the sialidase is a bacterial sialidase.

6. The engineered immune cell of claim 5, wherein the sialidase is selected from the group consisting of Clostridium perfringens sialidase, Actinomyces viscosus sialidase, Arthrobacter ureafaciens sialidase, and derivatives thereof.

7. The engineered immune cell of claim 6, wherein the sialidase is an Actinomyces viscosus sialidase or a derivative thereof.

8. The engineered immune cell of any one of claims 1-3, wherein the sialidase comprises an amino acid sequence having at least about 80% sequence identity to an amino acid sequence selected from the group consisting of SEQ ID NOs: 1-28, 31, and 53-45.

9. The engineered immune cell of claim 7, wherein the sialidase comprises an amino acid sequence having at least about 60% sequence identity to the amino acid sequence of SEQ ID NO: 1 or 2.

10. The engineered immune cell of claim 8, wherein the sialidase is DAS181.

11. The engineered immune cell of any one of the preceding claims, wherein the sialidase is membrane-associated.

12. The engineered immune cell of any one of claims 1-11, wherein the sialidase is secreted by the engineered immune cell.

13. The engineered immune cell of any one of the preceding claims, wherein the sialidase comprises an anchoring domain.

14. The engineered immune cell of claim 12, wherein the sialidase is a fusion protein comprising from the N-terminus to the C-terminus: a sialidase catalytic domain and an anchoring domain.

15. The engineered immune cell of claim 12 or 13, wherein the anchoring domain is positively charged at physiologic pH.

16. The engineered immune cell of any one of claims 12-14, wherein the anchoring domain is a glycosaminoglycan (GAG)-binding domain.

17. The engineered immune cell of any one of the preceding claims, wherein the first heterologous nucleotide sequence further encodes a secretion sequence operably linked to the sialidase.

18. The engineered immune cell of claim 15, wherein the secretion sequence comprises the amino acid sequence of SEQ ID NO: 40.

19. The engineered immune cell of any one of the preceding claims, wherein the sialidase comprises a transmembrane domain.

20. The engineered immune cell of claim 18, wherein the sialidase is a fusion protein comprising from the N-terminus to the C-terminus: a sialidase catalytic domain, a linker, and a transmembrane domain.

21. The engineered immune cell of any one of claims 12-18, wherein the anchoring domain or the transmembrane moiety is located at the carboxy terminus of the sialidase.

22. The engineered immune cell of any one of the preceding claims, wherein the sialidase is capable of cleaving both α-2,3 and α-2,6 sialic acid linkages.

23. The engineered immune cell of any one of claims 2-21, wherein the chimeric immune receptor is selected from the group consisting of a chimeric antigen receptor (CAR), an engineered T cell receptor (TCR), and a T cell receptor fusion protein (TFP).

24. The engineered immune cell of claim 22, wherein the chimeric immune receptor is a chimeric antigen receptor (CAR).

25. The engineered immune cell of claim 23, wherein the CAR comprises from the N-terminus to the C-terminus: an antigen-binding domain, a transmembrane domain, one or more co-stimulatory domains, and a primary signaling domain.

26. The engineered immune cell of any one of the preceding claims, wherein the engineered immune cell is a T-cell, a natural killer (NK) cell, a macrophage, or a natural killer T (NKT) cell.

27. The engineered immune cell of claim 25, wherein the engineered immune cell is a T cell.

28. The engineered immune cell of claim 25, wherein the engineered immune cell is an NK cell.

29. The engineered immune cell of any one of the preceding claims wherein the chimeric immune receptor specifically recognizes a tumor antigen.

30. The engineered immune cell of claim 28, wherein the tumor antigen is selected from the group consisting of carcinoembryonic antigen, alphafetoprotein, MUC16, survivin, glypican-3, B7 family members, VISTA, MICA/B, LILRB, CD19, BCMA, NY-ESO-1, CD20, CD22, CD24, CD33, CD38, CD200, CEA, EGFRvIII, Integrin beta 1, Integrin beta 4, GD2, HER2, IGF1R, mesothelin, PSMA, ROR1, WT1, NY-ESO-1, and CDH17.

31. The engineered immune cell of claim 29, wherein the tumor antigen is CD-19.

32. The engineered immune cell of claim 29, wherein the chimeric immune receptor specifically recognizes LILRB.

33. The engineered immune cell of any one of claims 1-27, wherein the engineered immune cell further comprises a third heterologous nucleotide sequence encoding a heterologous protein, wherein the heterologous protein is a secreted protein that promotes an inflammatory response or inhibits an immunoinhibitory molecule.

34. The engineered immune cell of claim 33, wherein the third heterologous nucleotide sequence encodes a heterologous protein that promotes an M2 to M1 switch in a macrophage population.

35. The engineered immune cell of any one of the preceding claims, wherein the first nucleotide sequence, the second nucleotide sequence, and/or the third nucleotide sequence are present in a lentiviral vector.

36. A pharmaceutical composition comprising the engineered immune cell of any one of the preceding claims and a pharmaceutically acceptable carrier.

37. A method of treating a cancer in an individual in need thereof, comprising administering to the individual an effective amount of the engineered immune cell of any one of claims 1-35 or the pharmaceutical composition of claim 36.

38. The method of claim 37, wherein the sialidase reduces sialylation of tumor cells.

39. A composition comprising a first engineered immune cell comprising a first heterologous nucleotide sequence encoding a sialidase, and a second engineered immune cell comprising a second heterologous nucleotide sequence encoding a chimeric immune receptor.


SEQUENCE LISTING (71 sequences)

SEQ ID NO: 1 - protein, 395 aa, Actinomyces viscosus
MGDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPAE

SEQ ID NO: 2 - protein, 415 aa, Artificial Sequence
  Synthetic Construct
MGDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPAKRKKKGGKNGKNRRNRKKKNP

SEQ ID NO: 3 - protein, 415 aa, Homo sapiens
MTGERPSTALPDRRWGPRILGFWGGCRVWVFAAIFLLLSLAASWSKAENDFGLVQPLVTMEQLLWVSGRQIGSVDTFRIPLITATPRGTLLAFAEARKMSSSDEGAKFIALRRSMDQGSTWSPTAFIVNDGDVPDGLNLGAVVSDVETGVVFLFYSLCAHKAGCQVASTMLVWSKDDGVSWSTPRNLSLDIGTEVFAPGPGSGIQKQREPRKGRLIVCGHGTLERDGVFCLLSDDHGASWRYGSGVSGIPYGQPKQENDFNPDECQPYELPDGSVVINARNQNNYHCHCRIVLRSYDACDTLRPRDVTFDPELVDPVVAAGAVVTSSGIVFFSNPAHPEFRVNLTLRWSFSNGTSWRKETVQLWPGPSGYSSLATLEGSMDGEEQAPQLYVLYEKGRNHYTESISVAKISVYGTL

SEQ ID NO: 4 - protein, 380 aa, Homo sapiens
MASLPVLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPIQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQ

SEQ ID NO: 5 - protein, 428 aa, Homo sapiens
MEEVTTCSFNSPLFRQEDDRGITYRIPALLYIPPTHTFLAFAEKRSTRRDEDALHLVLRRGLRIGQLVQWGPLKPLMEATLPGHRTMNPCPVWEQKSGCVFLFFICVRGHVTERQQIVSGRNAARLCFIYSQDAGCSWSEVRDLTEEVIGSELKHWATFAVGPGHGIQLQSGRLVIPAYTYYIPSWFFCFQLPCKTRPHSLMIYSDDLGVTWHHGRLIRPMVTVECEVAEVTGRAGHPVLYCSARTPNRCRAEALSTDHGEGFQRLALSRQLCEPPHGCQGSVVSFRPLEIPHRCQDSSSKDAPTIQQSSPGSSLRLEEEAGTPSESWLLYSHPTSRKQRVDLGIYLNQTPLEAACWSRPWILHCGPCGYSDLAALEEEGLFGCLFECGTKQECEQIAFRLFTHREILSHLQGDCTSPGRNPSQFKSN

SEQ ID NO: 6 - protein, 484 aa, Homo sapiens
MGVPRTPSRTVLFERERTGLTYRVPSLLPVPPGPTLLAFVEQRLSPDDSHAHRLVLRRGTLAGGSVRWGALHVLGTAALAEHRSMNPCPVHDAGTGTVFLFFIAVLGHTPEAVQIATGRNAARLCCVASRDAGLSWGSARDLTEEAIGGAVQDWATFAVGPGHGVQLPSGRLLVPAYTYRVDRRECFGKICRTSPHSFAFYSDDHGRTWRCGGLVPNLRSGECQLAAVDGGQAGSFLYCNARSPLGSRVQALSTDEGTSFLPAERVASLPETAWGCQGSIVGFPAPAPNRPRDDSWSVGPGSPLQPPLLGPGVHEPPEEAAVDPRGGQVPGGPFSRLQPRGDGPRQPGPRPGVSGDVGSWTLALPMPFAAPPQSPTWLLYSHPVGRRARLHMGIRLSQSPLDPRSWTEPWVIYEGPSGYSDLASIGPAPEGGLVFACLYESGARTSYDEISFCTFSLREVLENVPASPKPPNLGDKPRGCCWPS

SEQ ID NO: 7 - protein, 496 aa, Homo sapiens
MMSSAAFPRWLSMGVPRTPSRTVLFERERTGLTYRVPSLLPVPPGPTLLAFVEQRLSPDDSHAHRLVLRRGTLAGGSVRWGALHVLGTAALAEHRSMNPCPVHDAGTGTVFLFFIAVLGHTPEAVQIATGRNAARLCCVASRDAGLSWGSARDLTEEAIGGAVQDWATFAVGPGHGVQLPSGRLLVPAYTYRVDRRECFGKICRTSPHSFAFYSDDHGRTWRCGGLVPNLRSGECQLAAVDGGQAGSFLYCNARSPLGSRVQALSTDEGTSFLPAERVASLPETAWGCQGSIVGFPAPAPNRPRDDSWSVGPGSPLQPPLLGPGVHEPPEEAAVDPRGGQVPGGPFSRLQPRGDGPRQPGPRPGVSGDVGSWTLALPMPFAAPPQSPTWLLYSHPVGRRARLHMGIRLSQSPLDPRSWTEPWVIYEGPSGYSDLASIGPAPEGGLVFACLYESGARTSYDEISFCTFSLREVLENVPASPKPPNLGDKPRGCCWPS

SEQ ID NO: 8 - protein, 497 aa, Homo sapiens
MMSSAAFPRWLQSMGVPRTPSRTVLFERERTGLTYRVPSLLPVPPGPTLLAFVEQRLSPDDSHAHRLVLRRGTLAGGSVRWGALHVLGTAALAEHRSMNPCPVHDAGTGTVFLFFIAVLGHTPEAVQIATGRNAARLCCVASRDAGLSWGSARDLTEEAIGGAVQDWATFAVGPGHGVQLPSGRLLVPAYTYRVDRRECFGKICRTSPHSFAFYSDDHGRTWRCGGLVPNLRSGECQLAAVDGGQAGSFLYCNARSPLGSRVQALSTDEGTSFLPAERVASLPETAWGCQGSIVGFPAPAPNRPRDDSWSVGPGSPLQPPLLGPGVHEPPEEAAVDPRGGQVPGGPFSRLQPRGDGPRQPGPRPGVSGDVGSWTLALPMPFAAPPQSPTWLLYSHPVGRRARLHMGIRLSQSPLDPRSWTEPWVIYEGPSGYSDLASIGPAPEGGLVFACLYESGARTSYDEISFCTFSLREVLENVPASPKPPNLGDKPRGCCWPS

SEQ ID NO: 9 - protein, 913 aa, Actinomyces viscosus
MTSHSPFSRRRLPALLGSLPLAATGLIAAAPPAHAVPTSDGLADVTITQVNAPADGLYSVGDVMTFNITLTNTSGEAHSYAPASTNLSGNVSKCRWRNVPAGTTKTDCTGLATHTVTAEDLKAGGFTPQIAYEVKAVEYAGKALSTPETIKGATSPVKANSLRVESITPSSSQENYKLGDTVSYTVRVRSVSDKTINVAATESSFDDLGRQCHWGGLKPGKGAVYNCKPLTHTITQADVDAGRWTPSITLTATGTDGATLQTLTATGNPINVVGDHPQATPAPAPDASTELPASMSQAQHLAANTATDNYRIPAIPPPPMGTCSSPTTSARRTTATAAATTPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGPVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWCRDRGTISMSCDDGASWTTSKVFHEPFVGYTTIAVQSDGSIGLLSEDAHNGADYGGIWYRNFTMNWLGEQCGQKPAEPSPGRRRRRHPQRHRRRSRPRRPRRALSPRRHRHHPPRPSRALRPSRAGPGAGAHDRSEHGAHTGSCAQSAPEQTDGPTAAPAPETSSAPAAEPTQAPTVAPSVEPTQAPGAQPSSAPKPGATGRAPSVVNPKATGAATEPGTPSSSASPAPSRNAAPTPKPGMEPDEIDRPSDGTMAQPTGAPARRVPRRRRRRRPAAGCLARDQRAADPGPCGCRGCRRVPAAAGSPFEELNTRRAGHPALSTD

SEQ ID NO: 10 - protein, 793 aa, Actinomyces viscosus
MTTTKSSALRRLSALAGSLALAVTGIIAAAPPAHATPTSDGLADVTITQTHAPADGIYAVGDVMTFDITLTNTSGQARSFAPASTNLSGNVLKCRWSNVAAGATKTDCTGLATHTVTAEDLKAGGFTPQIAYEVKAVGYKGEALNKPEPVTGPTSQIKPASLKVESFTLASPKETYTVGDVVSYTVRIRSLSDQTINVAATDSSFDDLARQCHWGNLKPGQGAVYNCKPLTHTITQADADHGTWTPSITLAATGTDGAALQTLAATGEPLSVVVERPKADPAPAPDASTELPASMSDAQHLAENTATDNYRIPAITTAPNGDLLVSYDERPRDNGNNGGDSPNPNHIVQRRSTDGGKTWSAPSYIHQGVETGRKVGYSDPSYVVDNQTGTIFNFHVKSFDQGWGHSQAGTDPEDRSVIQAEVSTSTDNGWSWTHRTITADITRDNPWTARFAASGQGIQIHQGPHAGRLVQQYTIRTADGVVQAVSVYSDDHGQTWQAGTPTGTGMDENKVVELSDGSLMLNSRASDGTGFRKVATSTDGGQTWSEPVPDKNLPDSVDNAQIIRPFPNAAPSDPRAKVLLLSHSPNPRPWSRDRGTISMSCDNGASWVTGRVFNEKFVGYTTIAVQSDGSIGLLSEDGNYGGIWYRNFTMGWVGDQCSQPRPEPSPSPTPSAAPSAEPTSEPTTAPAPEPTTAPSSEPSVSPEPSSSAIPAPSQSSSATSGPSTEPDEIDRPSDGAMAQPTGGAGRPSTSVTGATSRNGLSRTGTNALLVLGVAAAAAAGGYLVLRIRRARTE

SEQ ID NO: 11 - protein, 1130 aa, Streptococcus oralis
MNYKSLDRKQRYGIRKFAVGAASVVIGTVVFGANPVLAQEQANAAGANTETVEPGQGLSELPKEASSGDLAHLDKDLAGKLAAAQDNGVEVDQDHLKKNESAESETPSSTETPAEEANKEEESEDQGAIPRDYYSRDLKNANPVLEKEDVETNAANGQRVDLSNELDKLKQLKNATVHMEFKPDASAPRFYNLFSVSSDTKENEYFTMSVLDNTALIEGRGANGEQFYDKYTDAPLKVRPGQWNSVTFTVEQPTTELPHGRVRLYVNGVLSRTSLKSGNFIKDMPDVNQAQLGATKRGNKTVWASNLQVRNLTVYDRALSPDEVQTRSQLFERGELEQKLPEGAKVTEKEDVFEGGRNNQPNKDGIKSYRIPALLKTDKGTLIAGTDERRLHHSDWGDIGMVVRRSSDNGKTWGDRIVISNPRDNEHAKHADWPSPVNIDMALVQDPETKRIFAIYDMFLESKAVFSLPGQAPKAYEQVGDKVYQVLYKQGESGRYTIRENGEVFDPQNRKTDYRVVVDPKKPAYSDKGDLYKGNELIGNIYFEYSEKNIFRVSNTNYLWMSYSDDDGKTWSAPKDITHGIRKDWMHFLGTGPGTGIALRTGPHKGRLVIPVYTTNNVSYLSGSQSSRVIYSDDHGETWQAGEAVNDNRPVGNQTIHSSTMNNPGAQNTESTVVQLNNGDLKLFMRGLTGDLQVATSHDGGATWDKEIKRYPQVKDVYVQMSAIHTMHEGKEYILLSNAGGPGRNNGLVHLARVEENGELTWLKHNPIQSGKFAYNSLQELGNGEYGLLYEHADGNQNDYTLSYKKFNWDFLSRDRISPKEAKVKYAIQKWPGIIAMEFDSEVLVNKAPTLQLANGKTATFMTQYDTKTLLFTIDPEDMGQRITGLAEGAIESMHNLPVSLAGSKLSDGINGSEAAIHEVPEFTGGVNAEEAAVAEIPEYTGPLATVGEEVAPTVEKPEFTGGVNAEEAPVAEMPEYTGPLSTVGEEVAPTVEKPEFTGGVNAVEAAVHELPEFKGGVNAVLAASNELPEYRGGANFVLAASNDLPEYIGGVNGAEAAVHELPEYKGDTNLVLAAADNKLSLGQDVTYQAPAAKQAGLPNTGSKETHSLISLGLAGVLLSLFAFGKKRKE

SEQ ID NO: 12 - protein, 305 aa, Streptococcus oralis
MSDLKKYEGVIPAFYACYDDQGEVSPERTRALVQYFIDKGVQGLYVNGSSGECIYQSVEDRKLILEEVMAVAKGKLTIIAHVACNNTKDSMELARHAESLGVDAIATIPPIYFRLPEYSVAKYWNDISAAAPNTDYVIYNIPQLAGVALTPSLYTEMLKNPRVIGVKNSSMPVQDIQTFVSLGGEDHIVFNGPDEQFLGGRLMGAKAGIGGTYGAMPELFLKLNQLIAEKDLETARELQYAINAIIGKLTSAHGNMYGVIKEVLKINEGLNIGSVRSPLTPVTEEDRPVVEAAAQLIRETKERFL

SEQ ID NO: 13 - protein, 1110 aa, Streptococcus mitis
MNQRHFDRKQRYGIRKFTVGAASVVIGAVVFGVAPALAQEAPSTNGETAGQSLPELPKEVETGNLTNLDKELADKLSTATDKGTEVNREELQANPGSEKAAETEASNETPATESEDEKEDGNIPRDFYARELENVNTVVEKEDVETNPSNGQRVDMKEELDKLKKLQNATIHMEFKPDASAPRFYNLFSVSSDTKVNEYFTMAILDNTAIVEGRDANGNQFYGDYKTAPLKIKPGEWNSVTFTVERPNADQPKGQVRVYVNGVLSRTSPQSGRFIKDMPDVNQVQIGTTKRTGKNFWGSNLKVRNLTVYDRALSPEEVKKRSQLFERGELEKKLPEGAKVTDKLDVFQGGENRKPNKDGIASYRIPALLKTDKGTLIAGADERRLHHSDWGDIGMVVRRSDDKGKTWGDRIVISNPRDNENARRAHAGSPVNIDMALVQDPKTKRIFSIFDMFVEGEAVRDLPGKAPQAYEQIGNKVYQVLYKKGEAGHYTIRENGEVFDPENRKTEYRVVVDPKKPAYSDKGDLYKGEELIGNVYFDYSDKNIFRVSNTNYLWMSYSDDDGKTWSAPKDITYGIRKDWMHFLGTGPGTGIALHSGPHKGRLVIPAYTTNNVSYLGGSQSSRVIYSDDHGETWHAGEAVNDNRPIGNQTIHSSTMNNPGAQNTESTVVQLNNGDLKLFMRGLTGDLQVATSKDGGATWEKDVKRYADVKDVYVQMSAIHTVQEGKEYIILSNAGGPGRYNGLVHVARVEANGDLTWIKHNPIQSGKFAYNSLQDLGNGEFGLLYEHATATQNEYTLSYKKFNWDFLSKDGVAPTKATVKNAVEMSKNVIALEFDSEVLVNQPPVLKLANGNFATFLTQYDSKTLLFAASKEDIGQEITEIIDGAIESMHNLPVSLEGAGVPGGKNGAKAAIHEVPEFTGAVNGEGTVHEDPAFEGGINGEEAAVHDVPDFSGGVNGEVAAIHEVPEFTGGINGEEAAKLELPSYEGGANAVEAAKSELPSYEGGANAVEAAKLELPSYESGAHEVQPASSNLPTLADSVNKAEAAVHKGKEYKANQSTAVQAMAQEHTYQAPAAQQHLLPKTGSEDKSSLAIVGFVGMFLGLLMIGKKRE

SEQ ID NO: 14 - protein, 1292 aa, Streptococcus mitis
MNQSSLNRKNRYGIRKFTIGVASVAIGSVLFGITPALAQETTTNIDVSKVETSLESGAPVSEPVTEVVSGDLNHLDKDLADKLALATNQGVDVNKHNLKEETSKPEGNSEHLPVESNTGSEESIEHHPAKIEGADDAVVPPRDFFARELTNVKTVFEREDLATNTGNGQRVDLAEELDQLKQLQNATIHMEFKPDANAPQFYNLFSVSSDKKKDEYFSMSVNKGTAMVEARGADGSHFYGSYSDAPLKIKPGQWNSVTFTVERPKADQPNGQVRLYVNGVLSRTNTKSGRFIKDMPDVNKVQIGATRRANQTMWGSNLQIRNLTVYNRALTIEEVKKRSHLFERNDLEKKLPEGAEVTEKKDIFESGRNNQPNGEGINSYRIPALLKTDKGTLIAGGDERRLHHFDYGDIGMVIRRSQDNGKTWGDKLTISNLRDNPEATDKTATSPLNIDMVLVQDPTTKRIFSIYDMFPEGRAVFGMPNQPEKAYEEIGDKTYQVLYKQGETERYTLRDNGEIFNSQNKKTEYRVVVNPTEAGFRDKGDLYKNQELIGNIYFKQSDKNPFRVANTSYLWMSYSDDDGKTWSAPKDITPGIRQDWMKFLGTGPGTGIVLRTGAHKGRILVPAYTTNNISHLGGSQSSRLIYSDDHGQTWHAGESPNDNRPVGNSVIHSSNMNKSSAQNTESTVLQLNNGDVKLFMRGLTGDLQVATSKDGGVTWEKTIKRYPEVKDAYVQMSAIHTMHDGKEYILLSNAAGPGRERKNGLVHLARVEENGELTWLKHNPIQNGEFAYNSLQELGGGEYGLLYEHRENGQNYYTLSYKKFNWDFVSKDLISPTEAKVSQAYEMGKGVFGLEFDSEVLVNRAPILRLANGRTAVFMTQYDSKTLLFAVDKKDIGQEITGIVDGSIESMHNLTVNLAGAGIPGGMNAAESVEHYTEEYTGVLGTSGVEGVPTISVPEYEGGVNSELALVSEKEDYRGGVNSASSVVTEVLEYTGPLSTVGSEDAPTVSVLEYEGGVNIDSPEVTEAPEYKEPIGTSGYELAPTVDKPAYTGTIEPLEKEENSGAIIEEGNVSYITENNNKPLENNNVTTSSIISESSKLKHTLKNATGSVQIHASEEVLKNVKDVKIQEVKVSSLSSLNYKAYDIQLNDASGKAVQPKGTVIVTFAAEQSVENVYYVDSKGNLHTLEFLQKDGEVTFETNHFSIYAMTFQLSLDNVVLDNHREDKNGEVNSASPKLLSINGHSQSSQLENKVSNNEQSKLPNTGEDKSISTVLLGFVGVILGAMIFYRRKDSEG

SEQ ID NO: 15 - protein, 1004 aa, Streptococcus mitis
  VARIANT 298: Xaa = Any Amino Acid
MDKKKIILTSLASVAVLGAALAASQPSLVKAEEQPTASQPAGETGTKSEVTSPEIKQAEADAKAAEAKVTEAQAKVDTTTPVADEAAKKLETEKKEADEADAAKTKAEEAKKTADDELAAAKEKAAEADAKAKEEAKKEEDAKKEEADSKEALTEALKQLPDNELLDKKAKEDLLKAVEAGDLKASDILAELADDDKKAEANKETEKKLRNKDQANEANVATTPAEEAKSKDQLPADIKAGIDKAEKADAARPASEKLQDKADDLGENVDELKKEADALKAEEDKKAETLKKQEDTLXEAKEALKSAKDNGFGEDITAPLEKAVTAIEKERDAAQNAFDQAASDTKAVADELNKLTDEYNKTLEEVKAAKEKEANEPAKPVEEEPAKPAEKTEAEKAAEAKTEADAKVAELQKKADEAKTKADEATAKATKEAEDVKAAEKAKEEADKAKTDAEAELAKAKEEAEKAKAKVEELKKEEKDNLEALKAALDQLEKDIDADATITNKEEAKKALGKEDILAAVEKGDLTAGDVLKELENQNATAEATKDQDPQADEIGATKQEGKPLSELPAADKEKLDAAYNKEASKPIVKKLQDIADDLVEKIEKLTKVADKDKADATEKAKAVEEKNAALDKQKETLDKAKAALETAKKNQADQAIQDGLQDAVTKLEASFASAKTAADEAQAKFDEVNEVVKAYKAAIDELTDDYNATLGHIENLKEVPKGEEPKDFSGGVNDDEAPSSTPNTNEFTGGANDADAPTAPNANEFAGGVNDEEAPTTENKPEFNGGVNDEEAPTVPNKPEGEAPKPTGENAKDAPVVKLPEFGANNPEIKKILDEIAKVKEQIKDGEENGSEDYYVEGLKERLADLEEAFDTLSKNLPAVNKVPEYTGPVTPENGQTQPAVNTPGGQQGGSSQQTPAVQQGGSGQQAPAVQQGGSNQQVPAVQQTNTPAVAGTSQDNTYQAPAAKEEDKKELPNTGGQESAALASVGFLGLLLGALPFVKRKN

SEQ ID NO: 16 - protein, 1145 aa, Streptococcus mitis
MKYRDFDRKRRYGIRKFAVGAASVVIGTVVFGANPVLAQEQANAAGANTETVEPGQGLSELPKEASSGDLAHLDKDLAGKLAAAQDNGVEVDQDHLKKNESAESETPSSTETPAEGTNKEEESEDQGAIPRDYYSRDLKNANPVLEKEDVETNAANGQRVDLSNELDKLKQLKNATVHMEFKPDASAPRFYNLFSVSSDTKENEYFTISVLDNTALIEGRGANGEQFYDKYTDAPLKVRPGQWNSVTFTVEQPTTELPHGRVRLYVNGVLSRTSLKSGNFIKDMPDVNQAQLGATKRGNKTVWASNLQVRNLTVYDRALSPDEVQTRSQLFERGELEQKLPEGAKVTEKEDVFEGGRNNQPNKDGIKSYRIPALLKTDKGTLIAGTDERRLHHSDWGDIGMVVRRSSDNGKTWGDRIVISNPRDNEHAKHADWPSPVNIDMALVQDPETKRIFAIYDMFLESKAVFSLPGQAPKAYEQVGDKVYQVLYKQGESGRYTIRENGEVFDPQNRKTDYRVVVDPKKPAYSDKGDLYKGNELIGNIYFEYSEKNIFRVSNTNYLWMSYSDDDGKTWSAPKDITHGIRKDWMHFLGTGPGTGIALRTGPHKGRLVIPVYTTNNVSYLSGSQSSRVIYSDDHGETWQAGEAVNDNRPVGNQTIHSSTMNNPGAQNTESTVVQLNNGDLKLFMRGLTGDLQVATSHDGGATWDKEIKRYPQVKDVYVQMSAIHTMHEGKEYILLSNAGGPGRNNGLVHLARVEENGELTWLKHNPIQSGKFAYNSLQDLGNGEYGLLYEHADGNQNDYTLSYKKFNWDFLTKDWISPKEAKVKYAIEKWPGILAMEFDSEVLVNKAPTLQLANGKTARFMTQYDTKTLLFTVDSEDMGQKVTGLAEGAIESMHNLPVSVAGTKLSNGMNGSEAAVHEVPEYTGPLGTAGEEPAPTVEKPEFTGGVNGEEAAVHEVPEYTGPLGTSGEEPAPTVEKPEFTGGVNAVEAAAHEVPEYTGPLGTSGKEPAPTVEKPEYTGGVNAVEAAVHEVPEYTGPLATVGEEAAPKVDKPEFTGGVNAVEAAVHELPEYTGGVNAADAAVHEIAEYKGADSLVTLAAEDYTYKAPLAQQTLPDTGNKESSLLASLGLTAFFLGLFAMGKKREK

SEQ ID NO: 17 - protein, 1797 aa, Streptococcus mitis
MEKIWREKSCRYSIRKLTVGTASVLLGAVFLASHTVSADTIKVKQNESTLEKTTAKTDTVTKTTESTEHTQPSEAIDHSKQVLANNSSSESKPTEAKVASATTNQASTEAIVKPNENKETEKQELPVTEQSNYQLNYDRPTAPSYDGWEKQALPVGNGEMGAKVFGLIGEERIQYNEKTLWSGGPRPDSTDYNGGNYRERYKILAEIRKALEDGDRQKAKRLAEQNLVGPNNAQYGRYLAFGDIFMVFNNQKKGLDTVTDYHRGLDITEATTTTSYTQDGTTFKRETFSSYPDDVTVTHLTQKGDKKLDFTVWNSLTEDLLANGDYSAEYSNYKSGHVTTDPNGILLKGTVKDNGLQFASYLGIKTDGKVTVHEDSLTITGASYATLLLSAKTNFAQNPKTNYRKDIDLEKTVKGIVEAAQGKYYETLKRNHIKDYQSLFNRVKLNLGGSNIAQTTKEALQTYNPTKGQKLEELFFQYGRYLLISSSRDRTDALPANLQGVWNAVDNPPWNADYHLNVNLQMNYWPAYMSNLAETAKPMINYIDDMRYYGRIAAKEYAGIESKDGQENGWLVHTQATPFGWTTPGWNYYWGWSPAANAWMMQNVYDYYKFTKDETYLKEKIYPMLKETAKFWNSFLHYDQASDRWVSSPSYSPEHGTITIGNTFDQSLVWQLFHDYMEVANHLNVDKDLVTEVKAKFDKLKPLHINKEGRIKEWYEEDSPQFTNEGIENNHRHVSHLVGLFPGTLFSKDQAEYLEAARATLNHRGDGGTGWSKANKINLWARLLDGNRAHRLLAEQLKYSTLENLWDTHAPFQIDGNFGATSGIAEMLLQSHTGYIAPLPALPDAWKDGQVSGLVARGNFEVSMQWKDKNLQSLSFLSNVGGDLVVDYPNIEASQVKVNGKPVKATVLKDGRIQLATQKGDVITFEHFSGRVTSLTAVRQNGVTAELTFNQVEGATHYVIQRQVKDESGQTSATREFVTNQTHFIDRSLDPQLAYTYTVKAMLGNVSTQVSEKANVETYNQLMDDRDSRIQYGSAFGNWADSELFGGTEKFADLSLGNYTDKDATATIPFNGVGIEIYGLKSSQLGIAEVKIDGKSVGELDFYTAGATEKGSLIGRFTGLSDGAHVMTITVKQEHKHRGSERSKISLDYFKVLPGQGTTIEKMDDRDSRIQYGSQFKDWSDTELYKSTEKYADINNSDPSTASEAQATIPFTGTGIRIYGLKTSALGKALVTLDGKEMPSLDFYTAGATQKATLIGEFTNLTDGNHILTLKVDPNSPAGRKKISLDSFDVIKSPAVSLDSPSIAPLKKGDKNISLTLPAGDWEAIAVTFPGIKDPLVLRRIDDNHLVTTGDQTVLSIQDNQVQIPIPDETNRKIGNAIEAYSIQGNTTSSPVVAVFTKKDEKKVENQQPTTSKGDDPAPIVEIPEYTKPIGTAGLEQPPTVSIPEYTQPIGTAGLEQAPTVSIPEYTKPVGTAGIEQAPTVSIPEYTKPIGTAGLEQAPTVSIPEYTQPIGTAGLEQPPTVSIPEYTKSIGTAGLEQPPVVNVPEYTQPIGTAGIEQPPTVSIPEYTKPIGTAGQEQALTVSIPEYTKPIGTAGQEQAPTVSVPEYKLRVLKDERTGVEIIGGATDLEGISHISSRRVLAQELFGKTYDAYDLHLKNSTDQSLQPKGSVLVRLPISSAVENVYYLTPSKELQALDFTIREGMAEFTTSHFSTYAVVYQANGASTTAEQKPSETDIKPLANSSEQVSSSPDLVQSTNDSPKEQLPATGETSNPLLFLSGLSLVLTATFLLKSKKDESN

SEQ ID NO: 18 - protein, 2115 aa, Streptococcus mitis
MKQYFLEKGRIFSIRKLTVGVASVAVGLTFFASGNVAASELVTEPKLEVDGQSKEVADVKHEKEEAVKEEAVKEEVTEKTELTAEKATEEAKTAEVAGDVLPEEIPDRAYPDTPVKKVDTAAIVSEQESPQVETKSILKPTEVAPTEGEKENRAVINGGQDLKRINYEGQPATSAAMVYTIFSSPLAGGGSQRYLNSGSGIFVAPNIMLTVAHNFLVKDADTNAGSIRGGDTTKFYYNVGSNTAKNNSLPTSGNTVLFKEKDIHFWNKEKFGEGIKNDLALVVAPVPLSIASPNKAATFTPLAEHREYKAGEPVSTIGYPTDSTSPELKEPIVPGQLYKADGVVKGTEKLDDKGAVGITYRLTSVSGLSGGGIINGDGKVIGIHQHGTVDNMNIAEKDRFGGGLVLSPEQLAWVKEIIDKYGVKGWYQGDNGNRYYFTPEGEMIRNKTAVIGKNKYSFDQNGIATLLEGVDYGRVVVEHLDQKDNPVKENDTFVEKTEVGTQFDYNYKTEIEKTDFYKKNKEKYEIVSIDGKAVNKQLKDTWGEDYSVVSKAPAGTRVIKVVYKVNKGSFDLRYRLKGTDQELAPATVDNNDGKEYEVSFVHRFQAKEITGYRAVNASQEATIQHKGVNQVIFEYEKIEDPKPATPATPVVDPKDEETEIGNYGPLPSKAQLDYHKEELAAFIHYGMNTYTNSEWGNGRENPQNFNPTNLDTDQWIKTLKDAGFKRTIMVVKHHDGFVIYPSQYTKHTVAASPWKDGKGDLLEEISKSATKYDMNMGVYLSPWDANNPKYHVSTEKEYNEYYLNQLKEILGNPKYGNKGKFIEVWMDGARGSGAQKVTYTFDEWFKYIKKAEGDIAIFSAQPTSVRWIGNERGIAGDPVWHKVKKAKITDDVKNEYLNHGDPEGDMYSVGEADVSIRSGWFYHDNQQPKSIKDLMDIYFKSVGRGTPLLLNIPPNKEGKFADADVARLKEFRATLDQMYATDFAKGATVTASSTRKNHLYQASNLTDGKDDTSWALSNDAKTGEFTVDLGQKRRFDVVELKEDIAKGQRISGFKVEVELNGRWVPYGEGSTVGYRRLVQGQPVEAQKIRVTITNSQATPILTNFSVYKTPSSIEKTDGYPLGLDYHSNTTADKANTTWYDESEGIRGTSMWTNKKDASVTYRFNGTKAYVVSTVDPNHGEMSVYVDGQKVADVQTNNAARKRSQMVYETDDLAPGEHTIKLVNKTGKAIATEGIYTLNNAGKGMFELKETTYEVQKGQPVTVTIKRVGGSKGAATVHVVTEPGTGVHGKVYKDTTADLTFQDGETEKTLTIPTIDFTEQADSIFDFKVKMTSASDNALLGFASEATVRVMKADLLQKDQVSHDDQASQLDYSPGWHHETNSAGKYQNTESWASFGRLNEEQKKNASVTAYFYGTGLEIKGFVDPGHGIYKVTLDGKELEYQDGQGNATDVNGKKYFSGTATTRQGDQTLVRLTGLEEGWHAVTLQLDPKRNDTSRNIGIQVDKFITRGEDSALYTKEELVQAMKNWKDELAKFDQTSLKNTPEARQAFKSNLDKLSEQLSASPANAQEILKIATALQAILDKEENYGKEDTPTSEQPEEPNYDKAMASLSEAIQNKSKELSSDKEAKKKLVELSEQALTAIQEAKTQDAVDKALQAALTSINQLQATPKEEVKPSQPEEPNYDKAMASLAEAIQNKSKELGSDKESKKKLVELSEQALTAIQEAKTQDAVDKALQAALTSINQLQATPKEEAKPSQPEEPNYDKAMASLAEAIQNKSKELGSDKEAKKKLVELSEQALTAIQEAKTQDAVDKALQAALTSINQLQATPKEEVKHSIVPTDGDKELVQPQPSLEVVEKVINFKKVKQEDSSLPKGETRVTQVGRAGKERILTEVAPDGSRTIKLREVVEVAQDEIVLVGTKKEESGKIASSVHEVPEFTGGVIDSEATIHNLPEFTGGVTDSEAAIHNLPEFTGGVTDSEAAIHNLPEFTGGMTDSEAAIHNLPEFTGGMTDSEGVAHGVSNVEEGVPSGEATSHQESGFTSDVTDSETTMNEIVYKNDEKSYVVPPMLEDKTYQAPANRQEVLPKTGSEDGSAFASVGIIGMFLGMIGIVKRKKD

SEQ ID NO: 19 - protein, 305 aa, Streptococcus mitis
MSGLKKYEGVIPAFYACYDDAGEVSPERTRALVQYFIDKGVQGLYVNGSSGECIYQSVEDRKLILEEVMAVAKGKLTIIAHVACNNTKDSIELARHAESLGVDAIATIPPIYFRLPEYSVAKYWNDISAAAPNTDYVIYNIPQLAGVALTPSLYTEMLKNPRVIGVKNSSMPVQDIQTFVSLGGDDHIVFNGPDEQFLGGRLMGAKAGIGGTYGAMPELFLKLNQLIADKDLETARELQYAINAIIGKLTAAHGNMYCVIKEVLKINEGLNIGSVRSPLTPVTEEDRPVVEAAAQLIRESKERFL

SEQ ID NO: 20 - protein, 526 aa, Porphyromonas gingivalis
MANNTLLAKTRRYVCLVVFCCLMAMMHLSGQEVTMWGDSHGVAPNQVRRTLVKVALSESLPPGAKQIRIGFSLPKETEEKVTALYLLVSDSLAVRDLPDYKGRVSYDSFPISKEDRTTALSADSVAGRCFFYLAADIGPVASFSRSDTLTARVEELAVDGRPLPLKELSPASRRLYREYEALFVPGDGGSRNYRIPSILKTANGTLIAMADRRKYNQTDLPEDIDIVMRRSTDGGKSWSDPRIIVQGEGRNHGFGDVALVQTQAGKLLMIFVGGVGLWQSTPDRPQRTYISESRDEGLTWSPPRDITHFIFGKDCADPGRSRWLASFCASGQGLVLPSGRVMFVAAIRESGQEYVLNNYVLYSDDEGGTWQLSDCAYHRGDEAKLSLMPDGRVLMSVRNQGRQESRQRFFALSSDDGLTWERAKQFEGIHDPGCNGAMLQVKRNGRNQMLHSLPLGPDGRRDGAVYLFDHVSGRWSAPVVVNSGSSAYSDMTLLADGTIGYFVEEDDEISLVFIRFVLDDLFDARQ

SEQ ID NO: 21 - protein, 465 aa, Tannerella forsythia
MTKKSSISRRSFLKSTALAGAAGMVGTGGAATLLTSCGGGASSNENANAANKPLKEPGTYYVPELPDMAADGKELKAGIIGCGGRGSGAAMNFLAAANGVSIVALGDTFQDRVDSLAQKLKDEKNIDIPADKRFVGLDAYKQVIDSDVDVVIVATPPNFRPIHFQYAVEKSKHCFLEKPICVDAVGYRTIMATAKQAQAKNLCVITGTQRHHQRSYIASYQQIMNGAIGEITGGTVYWNQSMLWYRERQAGWSDCEWMIRDWVNWKWLSGDHIVEQHVHNIDVFTWFSGLKPVKAVGFGSRQRRITGDQYDNFSIDFTMENGIHLHSMCRQIDGCANNVSEFIQGTKGSWNSTDMGIKDLAGNVIWKYDVEAEKASFKQNDPYTLEHVNWINTIRAGKSIDQASETAVSNMAAIMGRESAYTGEETTWEAMTAAALDYTPADLNLGKMDMKPFVVPVPGKPLEKK

SEQ ID NO: 22 - protein, 539 aa, Tannerella forsythia
MKKFFWIIGLFISMLTTRAADSVYVQNPQIPILIDRTDNVLFRIRIPDATKGDVLNRLTIRFGNEDKLSEVKAVRLFYAGTEAGTKGRSRFAPVTYVSSHNIRNTRSANPSYSVRQDEVTTVANTLTLKTRQPMVKGINYFWVSVEMDRNTSLLSKLTPTVTEAVINDKPAVIAGEQAAVRRMGIGVRHAGDDGSASFRIPGLVTTNEGTLLGVYDVRYNNSVDLQEHIDVGLSRSTDKGQTWEPMRIAMSFGETDGLPSGQNGVGDPSILVDERTNTVWVVAAWTHGMGNARAWTNSMPGMTPDETAQLMMVKSTDDGRTWSEPINITSQVKDPSWCFLLQGPGRGITMRDGTLVFPIQFIDSLRVPHAGIMYSKDRGETWHIHQPARTNTTEAQVAEVEPGVLMLNMRDNRGGSRAVSITRDLGKSWTEHSSNRSALPESICMASLISVKAKDNIIGKDLLFFSNPNTTEGRHHITIKASLDGGVTWLPAHQVLLDEEDGWGYSCLSMIDRETVGIFYESSVAHMTFQAVKIKDLIR

SEQ ID NO: 23 - protein, 419 aa, Akkermansia muciniphila
MTWLLCGRGKWNKVKRMMNSVFKCLMSAVCAVALPAFGQEEKTGFPTDRAVTVFSAGEGNPYASIRIPALLSIGKGQLLAFAEGRYKNTDQGENDIIMSVSKNGGKTWSRPRAIAKAHGATFNNPCPVYDAKTRTVTVVFQRYPAGVKERQPNIPDGWDDEKCIRNFMIQSRNGGSSWTKPQEITKTTKRPSGVDIMASGPNAGTQLKSGAHKGRLVIPMNEGPFGKWVISCIYSDDGGKSWKLGQPTANMKGMVNETSIAETDNGGVVMVARHWGAGNCRRIAWSQDGGETWGQVEDAPELFCDSTQNSLMTYSLSDQPAYGGKSRILFSGPSAGRRIKGQVAMSYDNGKTWPVKKLLGEGGFAYSSLAMVEPGIVGVLYEENQEHIKKLKFVPITMEWLTDGEDTGLAPGKKAPVLK

SEQ ID NO: 24 - protein, 674 aa, Akkermansia muciniphila
MGLGLLCALGLSIPSVLGKESFEQARRGKFTTLSTKYGLMSCRNGVAEIGGGGKSGEASLRMFGGQDAELKLDLKDTPSREVRLSAWAERWTGQAPFEFSIVAIGPNGEKKIYDGKDIRTGGFHTRIEASVPAGTRSLVFRLTSPENKGMKLDDLFLVPCIPMKVNPQVEMASSAYPVMVRIPCSPVLSLNVRTDGCLNPQFLTAVNLDFTGTTKLSDIESVAVIRGEEAPIIHHGEEPFPKDSSQVLGTVKLAGSARPQISVKGKMELEPGDNYLWACVTMKEGASLDGRVVVRPASVVAGNKPVRVANAAPVAQRIGVAVVRHGDFKSKFYRIPGLARSRKGTLLAVYDIRYNHSGDLPANIDVGVSRSTDGGRTWSDVKIAIDDSKIDPSLGATRGVGDPAILVDEKTGRIWVAAIWSHRHSIWGSKSGDNSPEACGQLVLAYSDDDGLTWSSPINITEQTKNKDWRILFNGPGNGICMKDGTLVFAAQYWDGKGVPWSTIVYSKDRGKTWHCGTGVNQQTTEAQVIELEDGSVMINARCNWGGSRIVGVTKDLGQTWEKHPTNRTAQLKEPVCQGSLLAVDGVPGAGRVVLFSNPNTTSGRSHMTLKASTNDAGSWPEDKWLLYDARKGWGYSCLAPVDKNHVGVLYESQGALNFLKIPYKDVLNAKNAR

SEQ ID NO: 25 - protein, 544 aa, Bacteroides thetaiotaomicron
MKRNHYLFTLILLLGCSIFVKASDTVFVHQTQIPILIERQDNVLFYFRLDAKESRMMDEIVLDFGKSVNLSDVQAVKLYYGGTEALQDKGKKRFAPVDYISSHRPGNTLAAIPSYSIKCAEALQPSAKVVLKSHYKLFPGINFFWISLQMKPETSLFTKISSELQSVKIDGKEAICEERSPKDIIHRMAVGVRHAGDDGSASFRIPGLVTSNKGTLLGVYDVRYNSSVDLQEYVDVGLSRSTDGGKTWEKMRLPLSFGEYDGLPAAQNGVGDPSILVDTQTNTIWVVAAWTHGMGNQRAWWSSHPGMDLYQTAQLVMAKSTDDGKTWSKPINITEQVKDPSWYFLLQGPGRGITMSDGTLVFPTQFIDSTRVPNAGIMYSKDRGKTWKMHNMARTNTTEAQVVETEPGVLMLNMRDNRGGSRAVAITKDLGKTWTEHPSSRKALQEPVCMASLIHVEAEDNVLDKDILLFSNPNTTRGRNHITIKASLDDGLTWLPEHQLMLDEGEGWGYSCLTMIDRETIGILYESSAAHMTFQAVKLKDLIR

SEQ ID NO: 26 - protein, 911 aa, Actinomyces viscosus
MTSHSPFSRRHLPALLGSLPLAATGLIAAAPPAHAVPTSDGLADVTITQVNAPADGLYSVGDVMTFNITLTNTSGEAHSYAPASTNLSGNVSKCRWRNVPAGTTKTDCTGLATHTVTAEDLKAGGFTPQIAYEVKAVEYAGKALSTPETIKGATSPVKANSLRVESITPSSSKEYYKLGDTVTYTVRVRSVSDKTINVAATESSFDDLGRQCHWGGLKPGKGAVYNCKPLTHTITQADVDAGRWTPSITLTATGTDGTALQTLTATGNPINVVGDHPQATPAPAPDASTELPASMSQAQHVAPNTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDHGWGNSQAGTDPENRGIIQAEVSTSTDNGWTWTHRTITADITKDNPWTARFAASGQGIQIQHGPHAGRLVQQYTIRTAGGAVQAVSVYSDDHGKTWQAGTPVGTGMDENKVVELSDGSLMLNSRASDSSGFRKVAHSTDGGQTWSEPVSDKNLPDSVDNAQIIRAFPNAAPDDPRAKVLLLSHSPNPKPWSRDRGTISMSCDDGASWTTSKVFHEPFVGYTTIAVQSDGSIGLLSEDAHDGANYGGIWYRNFTMNWLGEQCGQKPAEPSPAPSPTAAPSAAPSEQPAPSAAPSTEPTQAPAPSSAPEPSAVPEPSSAPAPEPTTAPSTEPTPTPAPSSAPEPSAGPTAAPAPETSSAPAAEPTQAPTVAPSAEPTQVPGAQPSAAPSEKPGAQPSSAPKPDATGRAPSVVNPKATAAPSGKASSSASPAPSRSATATSKPGMEPDEIDRPSDGAMAQPTGGASAPSAAPTQAAKAGSRLSRTGTNALLVLGLAGVAVVGGYLLLRARRSKN

SEQ ID NO: 27 - protein, 393 aa, Artificial Sequence
  Synthetic Construct
GDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPA

SEQ ID NO: 28 - protein, 435 aa, Artificial Sequence
  Synthetic Construct
METDTLLLWVLLLWVPGSTGDGDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPAKRKKKGGKNGKNRRNRKKKNP

SEQ ID NO: 29 - protein, 435 aa, Artificial Sequence
  Synthetic Construct
METDTLLLWVLLLWVPGSTGDGDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGFTTIAVQSDGSIGLLSEDAHNGADYGGIWYRNFTMNWLGEQCGQKPAKRKKKGGKNGKNRRNRKKKNP

SEQ ID NO: 30 - protein, 422 aa, Artificial Sequence
  Synthetic Construct
METDTLLLWVLLLWVPGSTGDMASLPVLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPIQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQKRKKKGGKNGKNRRNRKKKNP

SEQ ID NO: 31 - protein, 495 aa, Artificial Sequence
  Synthetic Construct
METDTLLLWVLLLWVPGSTGDYPYDVPDYAGATPARSPGMGDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGFTTIAVQSDGSIGLLSEDAHNGADYGGIWYRNFTMNWLGEQCGQKPAVDEQKLISEEDLNAVGQDTQEVIVVPHSLPFKVVVISAILALVVLTIISLIILIMLWQKKPR

SEQ ID NO: 32 - protein, 495 aa, Artificial Sequence; identical to SEQ ID NO: 31
  Synthetic Construct

SEQ ID NO: 33 - protein, 481 aa, Artificial Sequence
  Synthetic Construct
METDTLLLWVLLLWVPGSTGDYPYDVPDYAGATPARSPGMASLPVLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPIQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQVDEQKLISEEDLNAVGQDTQEVIVVPHSLPFKVVVISAILALVVLTIISLIILIMLWQKKPR

SEQ ID NO: 34 - DNA, 1311 bp, Artificial Sequence
  Synthetic Construct
atggagacagacacactcctgctatgggtactgctgctctgggttccaggttccactggtgacggcgaccacccacaggcaacaccagcacctgccccagatgcctccaccgagctgccagcaagcatgtcccaggcacagcacctggcagcaaataccgcaacagacaactacagaatccccgccatcaccacagccccaaatggcgatctgctgatcagctatgacgagcgccccaaggataacggaaatggaggctccgacgcaccaaaccctaatcacatcgtgcagcggagatctaccgatggcggcaagacatggagcgcccctacctacatccaccagggcaccgagacaggcaagaaggtcggctactctgacccaagctatgtggtggatcaccagaccggcacaatcttcaactttcacgtgaagtcctatgaccagggatggggaggctctaggggcggcaccgatcctgagaatcgcggcatcatccaggccgaggtgtctaccagcacagacaacggctggacctggacacaccggaccatcacagccgacatcacaaaggataagccctggaccgcaagattcgcagcaagcggacagggcatccagatccagcacggacctcacgcaggccggctggtgcagcagtacaccatcagaacagcaggaggagcagtgcaggccgtgtccgtgtattctgacgatcacggcaagacctggcaggcaggcaccccaatcggcacaggcatggacgagaataaggtggtggagctgagcgatggctccctgatgctgaactctagggccagcgacggctccggcttccgcaaggtggcacactctacagacggaggacagacctggtccgagcccgtgtctgataagaatctgcctgacagcgtggataacgcccagatcatccgggcctttcctaatgccgccccagacgatcccagagccaaggtgctgctgctgtcccactctccaaacccaaggccttggagccgggacagaggcacaatcagcatgtcctgcgacgatggcgccagctggaccacatccaaggtgttccacgagccatttgtgggctacaccacaatcgccgtgcagtctgatggcagcatcggactgctgagcgaggacgcacacaatggcgccgattacggcggcatctggtatcggaacttcaccatgaactggctgggcgagcagtgtggccagaagccagccaagcggaagaagaagggcggcaagaacggcaagaataggcgcaaccggaagaagaagaacccctgatga

SEQ ID NO: 35 - DNA, 1311 bp, Artificial Sequence
  Synthetic Construct
atggagacagacacactcctgctatgggtactgctgctctgggttccaggttccactggtgacggcgaccacccacaggcaacaccagcacctgccccagatgcctccaccgagctgccagcaagcatgtcccaggcacagcacctggcagcaaataccgcaacagacaactacagaatccccgccatcaccacagccccaaatggcgatctgctgatcagctatgacgagcgccccaaggataacggaaatggaggctccgacgcaccaaaccctaatcacatcgtgcagcggagatctaccgatggcggcaagacatggagcgcccctacctacatccaccagggcaccgagacaggcaagaaggtcggctactctgacccaagctatgtggtggatcaccagaccggcacaatcttcaactttcacgtgaagtcctatgaccagggatggggaggctctaggggcggcaccgatcctgagaatcgcggcatcatccaggccgaggtgtctaccagcacagacaacggctggacctggacacaccggaccatcacagccgacatcacaaaggataagccctggaccgcaagattcgcagcaagcggacagggcatccagatccagcacggacctcacgcaggccggctggtgcagcagtacaccatcagaacagcaggaggagcagtgcaggccgtgtccgtgtattctgacgatcacggcaagacctggcaggcaggcaccccaatcggcacaggcatggacgagaataaggtggtggagctgagcgatggctccctgatgctgaactctagggccagcgacggctccggcttccgcaaggtggcacactctacagacggaggacagacctggtccgagcccgtgtctgataagaatctgcctgacagcgtggataacgcccagatcatccgggcctttcctaatgccgccccagacgatcccagagccaaggtgctgctgctgtcccactctccaaacccaaggccttggagccgggacagaggcacaatcagcatgtcctgcgacgatggcgccagctggaccacatccaaggtgttccacgagccatttgtgggcttcaccacaatcgccgtgcagtctgatggcagcatcggactgctgagcgaggacgcacacaatggcgccgattacggcggcatctggtatcggaacttcaccatgaactggctgggcgagcagtgtggccagaagccagccaagcggaagaagaagggcggcaagaacggcaagaataggcgcaaccggaagaagaagaacccctgatga

SEQ ID NO: 36 - DNA, 1272 bp, Artificial Sequence
  Synthetic Construct
atggagacagacacactcctgctatgggtactgctgctctgggttccaggttccactggtgacatggccagcctgcctgtgctgcagaaggagagcgtgttccagtccggcgcccacgcatacagaatccccgccctgctgtatctgcctggccagcagtccctgctggcctttgccgagcagagagcctctaagaaggacgagcacgcagagctgatcgtgctgaggaggggcgactacgatgcaccaacccaccaggtgcagtggcaggcacaggaggtggtggcacaggcaaggctggacggacaccgcagcatgaatccatgccccctgtatgatgcccagaccggcacactgttcctgttctttatcgcaatccccggccaggtgaccgagcagcagcagctgcagaccagagccaacgtgacaagactgtgccaggtgacctccacagaccacggcaggacctggagcagccctcgcgacctgacagatgcagcaatcggaccagcatacagggagtggtctacattcgccgtgggccctggccactgcctgcagctgcacgatcgggccagaagcctggtggtgccagcctacgcctatcggaagctgcaccccatccagagacctatcccatctgccttctgctttctgagccacgaccacggcagaacttgggccagaggccactttgtggcccaggatacactggagtgtcaggtggcagaggtggagaccggagagcagagggtggtgacactgaatgcacgcagccacctgagggcccgcgtgcaggcccagtccaccaacgacggcctggatttccaggagtctcagctggtgaagaagctggtggagccacctccacagggatgtcagggctctgtgatcagctttccctcccctcggtctggcccaggcagcccagcacagtggctgctgtacacccaccccacacactcctggcagagggcagacctgggagcatatctgaatccaagaccccctgcaccagaggcctggtccgagcctgtgctgctggccaagggctcttgcgcctacagcgacctgcagagcatgggcaccggacctgatggctctccactgttcggctgtctgtacgaggccaacgattatgaggagatcgtgttcctgatgtttacactgaagcaggcctttcctgccgagtatctgccacagaagcggaagaagaagggcggcaagaacggcaagaatcggagaaaccggaagaagaagaacccttgatga

SEQ ID NO: 37 - DNA, 1485 bp, Artificial Sequence
  Synthetic Construct
atggagacagacacactcctgctatgggtactgctgctctgggttccaggttccactggtgactatccatatgatgttccagattatgctggggccacgccggccagatctcccgggatgggcgaccacccacaggcaacaccagcacctgccccagatgcctccaccgagctgccagcaagcatgtcccaggcacagcacctggcagcaaataccgcaacagacaactacagaatccccgccatcaccacagccccaaatggcgatctgctgatcagctatgacgagcgccccaaggataacggaaatggaggctccgacgcaccaaaccctaatcacatcgtgcagcggagatctaccgatggcggcaagacatggagcgcccctacctacatccaccagggcaccgagacaggcaagaaggtcggctactctgacccaagctatgtggtggatcaccagaccggcacaatcttcaactttcacgtgaagtcctatgaccagggatggggaggctctaggggcggcaccgatcctgagaatcgcggcatcatccaggccgaggtgtctaccagcacagacaacggctggacctggacacaccggaccatcacagccgacatcacaaaggataagccctggaccgcaagattcgcagcaagcggacagggcatccagatccagcacggacctcacgcaggccggctggtgcagcagtacaccatcagaacagcaggaggagcagtgcaggccgtgtccgtgtattctgacgatcacggcaagacctggcaggcaggcaccccaatcggcacaggcatggacgagaataaggtggtggagctgagcgatggctccctgatgctgaactctagggccagcgacggctccggcttccgcaaggtggcacactctacagacggaggacagacctggtccgagcccgtgtctgataagaatctgcctgacagcgtggataacgcccagatcatccgggcctttcctaatgccgccccagacgatcccagagccaaggtgctgctgctgtcccactctccaaacccaaggccttggagccgggacagaggcacaatcagcatgtcctgcgacgatggcgccagctggaccacatccaaggtgttccacgagccatttgtgggctacaccacaatcgccgtgcagtctgatggcagcatcggactgctgagcgaggacgcacacaatggcgccgattacggcggcatctggtatcggaacttcaccatgaactggctgggcgagcagtgtggccagaagccagccgtcgacgaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgt

SEQ ID NO: 38 - DNA, 1485 bp, Artificial Sequence
  Synthetic Construct
atggagacagacacactcctgctatgggtactgctgctctgggttccaggttccactggtgactatccatatgatgttccagattatgctggggccacgccggccagatctcccgggatgggcgaccacccacaggcaacaccagcacctgccccagatgcctccaccgagctgccagcaagcatgtcccaggcacagcacctggcagcaaataccgcaacagacaactacagaatccccgccatcaccacagccccaaatggcgatctgctgatcagctatgacgagcgccccaaggataacggaaatggaggctccgacgcaccaaaccctaatcacatcgtgcagcggagatctaccgatggcggcaagacatggagcgcccctacctacatccaccagggcaccgagacaggcaagaaggtcggctactctgacccaagctatgtggtggatcaccagaccggcacaatcttcaactttcacgtgaagtcctatgaccagggatggggaggctctaggggcggcaccgatcctgagaatcgcggcatcatccaggccgaggtgtctaccagcacagacaacggctggacctggacacaccggaccatcacagccgacatcacaaaggataagccctggaccgcaagattcgcagcaagcggacagggcatccagatccagcacggacctcacgcaggccggctggtgcagcagtacaccatcagaacagcaggaggagcagtgcaggccgtgtccgtgtattctgacgatcacggcaagacctggcaggcaggcaccccaatcggcacaggcatggacgagaataaggtggtggagctgagcgatggctccctgatgctgaactctagggccagcgacggctccggcttccgcaaggtggcacactctacagacggaggacagacctggtccgagcccgtgtctgataagaatctgcctgacagcgtggataacgcccagatcatccgggcctttcctaatgccgccccagacgatcccagagccaaggtgctgctgctgtcccactctccaaacccaaggccttggagccgggacagaggcacaatcagcatgtcctgcgacgatggcgccagctggaccacatccaaggtgttccacgagccatttgtgggcttcaccacaatcgccgtgcagtctgatggcagcatcggactgctgagcgaggacgcacacaatggcgccgattacggcggcatctggtatcggaacttcaccatgaactggctgggcgagcagtgtggccagaagccagccgtcgacgaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgt

SEQ ID NO: 39 - DNA, 1443 bp, Artificial Sequence
  Synthetic Construct
atggagacagacacactcctgctatgggtactgctgctctgggttccaggttccactggtgactatccatatgatgttccagattatgctggggccacgccggccagatctcccgggatggccagcctgcctgtgctgcagaaggagagcgtgttccagtccggcgcccacgcatacagaatccccgccctgctgtatctgcctggccagcagtccctgctggcctttgccgagcagagagcctctaagaaggacgagcacgcagagctgatcgtgctgaggaggggcgactacgatgcaccaacccaccaggtgcagtggcaggcacaggaggtggtggcacaggcaaggctggacggacaccgcagcatgaatccatgccccctgtatgatgcccagaccggcacactgttcctgttctttatcgcaatccccggccaggtgaccgagcagcagcagctgcagaccagagccaacgtgacaagactgtgccaggtgacctccacagaccacggcaggacctggagcagccctcgcgacctgacagatgcagcaatcggaccagcatacagggagtggtctacattcgccgtgggccctggccactgcctgcagctgcacgatcgggccagaagcctggtggtgccagcctacgcctatcggaagctgcaccccatccagagacctatcccatctgccttctgctttctgagccacgaccacggcagaacttgggccagaggccactttgtggcccaggatacactggagtgtcaggtggcagaggtggagaccggagagcagagggtggtgacactgaatgcacgcagccacctgagggcccgcgtgcaggcccagtccaccaacgacggcctggatttccaggagtctcagctggtgaagaagctggtggagccacctccacagggatgtcagggctctgtgatcagctttccctcccctcggtctggcccaggcagcccagcacagtggctgctgtacacccaccccacacactcctggcagagggcagacctgggagcatatctgaatccaagaccccctgcaccagaggcctggtccgagcctgtgctgctggccaagggctcttgcgcctacagcgacctgcagagcatgggcaccggacctgatggctctccactgttcggctgtctgtacgaggccaacgattatgaggagatcgtgttcctgatgtttacactgaagcaggcctttcctgccgagtatctgccacaggtcgacgaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgt

SEQ ID NO: 40 - protein, 21 aa, Homo sapiens
METDTLLLWVLLLWVPGSTGD

SEQ ID NO: 41 - protein, 9 aa, Artificial Sequence
  Synthetic Construct
YPYDVPDYA

SEQ ID NO: 42 - protein, 9 aa, Artificial Sequence
  Synthetic Construct
GATPARSPG

SEQ ID NO: 43 - protein, 2 aa, Artificial Sequence
  Synthetic Construct
VD

SEQ ID NO: 44 - protein, 10 aa, Artificial Sequence
  Synthetic Construct
EQKLISEEDL

SEQ ID NO: 45 - protein, 50 aa, Homo sapiens
NAVGQDTQEVIVVPHSLPFKVVVISAILALVVLTIISLIILIMLWQKKPR

SEQ ID NO: 46 - protein, 27 aa, Homo sapiens
FWVLVVVGGVLACYSLLVTVAFIIFWV

SEQ ID NO: 47 - protein, 22 aa, Homo sapiens
MALIVLGGVAGLLLFIGLGIFF

SEQ ID NO: 48 - protein, 21 aa, Homo sapiens
IYIWAPLAGTCGVLLLSLVIT

SEQ ID NO: 49 - protein, 23 aa, Homo sapiens
IYIWAPLAGTCGVLLLSLVITLY

SEQ ID NO: 50 - protein, 24 aa, Homo sapiens
IYIWAPLAGTCGVLLLSLVITLYC

SEQ ID NO: 51 - protein, 27 aa, Homo sapiens
IISFFLALTSTALLFLLFFLTLRFSVV

SEQ ID NO: 52 - protein, 21 aa, Homo sapiens
VVISAILALVVLTIISLIILI

SEQ ID NO: 53 - protein, 381 aa, Salmonella typhimurium
TVEKSVVFKAEGEHFTDQKGNTIVGSGSGGTTKYFRIPAMCTTSKGTIVVFADARHNTASDQSFIDTAAARSTDGGKTWNKKIAIYNDRVNSKLSRVMDPTCIVANIQGRETILVMVGKWNNNDKTWGAYRDKAPDTDWDLVLYKSTDDGVTFSKVETNIHDIVTKNGTISAMLGGVGSGLQLNDGKLVFPVQMVRTKNITTVLNTSFIYSTDGITWSLPSGYCEGFGSENNIIEFNASLVNNIRNSGLRRSFETKDFGKTWTEFPPMDKKVDNRNHGVQGSTITIPSGNKLVAAHSSAQNKNNDYTRSDISLYAHNLYSGEVKLIDDFYPKVGNASGAGYSCLSYRKNVDKETLYVVYEANGSIEFQDLSRHLPVIKSYN

SEQ ID NO: 54 - protein, 781 aa, Vibrio cholera
MRFKNVKKTALMLAMFGMATSSNAALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGDVIFRGPDRIPSIVASSVTPGVVTAFAEKRVGGGDPGALSNTNDIITRTSRDGGITWDTELNLTEQINVSDEFDFSDPRPIYDPSSNTVLVSYARWPTDAAQNGDRIKPWMPNGIFYSVYDVASGNWQAPIDVTDQVKERSFQIAGWGGSELYRRNTSLNSQQDWQSNAKIRIVDGAANQIQVADGSRKYVVTLSIDESGGLVANLNGVSAPIILQSEHAKVHSFHDYELQYSALNHTTTLFVDGQQITTWAGEVSQENNIQFGNADAQIDGRLHVQKIVLTQQGHNLVEFDAFYLAQQTPEVEKDLEKLGWTKIKTGNTMSLYGNASVNPGPGHGITLTRQQNISGSQNGRLIYPAIVLDRFFLNVMSIYSDDGGSNWQTGSTLPIPFRWKSSSILETLEPSEADMVELQNGDLLLTARLDFNQIVNGVNYSPRQQFLSKDGGITWSLLEANNANVFSNISTGTVDASITRFEQSDGSHFLLFTNPQGNPAGTNGRQNLGLWFSFDEGVTWKGPIQLVNGASAYSDIYQLDSENAIVIVETDNSNMRILRMPITLLKQKLTLSQN

SEQ ID NO: 55 - DNA, 1520 bp, Artificial Sequence
  Synthetic Construct
atggagtttggactgagctggctgtttctcgtggccattctgaagggcgtccagtgcagcagagacatccagatgacccagacaaccagctctctgagcgctagcctcggagatagagtgaccattagctgtagagcctcccaagacatttccaagtacctcaactggtaccagcagaagcccgacggcaccgtgaagctgctgatctaccacaccagcagactgcactccggagtgccctctaggttttccggatccggcagcggcacagactactctctgaccatctccaatctggagcaagaggacatcgccacctacttctgccagcaaggcaacacactgccttacacattcggcggcggaacaaagctcgaactgaaaagaggcggcggcggaagcggaggaggaggatccggaggcggaggatccggcggaggaggctccgaagtccagctgcaacaaagcggacccggactggtggctcccagccaatctctgagcgtgacatgcacagtgtccggcgtctctctgcccgactacggagtcagctggattagacagcctcctagaaagggactggagtggctgggagtcatctggggcagcgagaccacctactataactccgccctcaagtctaggctcaccatcatcaaagacaacagcaagagccaagtgttcctcaagatgaacagcctccagaccgacgacaccgccatctactactgcgccaaacactactactacggaggcagctacgctatggattactggggccaaggcaccacagtcacagtgagcagctatgtgaccgtgagcagccaagaccccgccaaagatcccaagttctgggtgctggtcgtggtgggaggcgtgctggcttgttattctctgctggtgaccgtggccttcatcatcttctgggtgaggagcaagagatccagactgctgcacagcgactacatgaacatgacacctagaaggcccggccccacaaggaaacattaccagccctacgccccccctagagacttcgctgcctatagatccaagagaggaagaaaaaagctgctctacatcttcaagcagcccttcatgaggcccgtgcaaacaacacaagaggaggacggatgtagctgtagattccccgaggaggaagagggaggatgcgagctgagagtgaagttctctaggagcgccgatgctcccgcttatcagcaaggccagaaccagctgtacaatgagctgaatctgggaagaagggaagaatacgacgtgctggataagaggaggggaagagaccccgagatgggaggcaagcctagaaggaagaacccccaagagggactgtacaacgagctccaaaaggacaagatggctgaagcctacagcgagatcggaatgaagggagagagaaggaggggcaagggccacgatggactctaccaaggcctcagcacagccaccaaggacacctacgacgctctgcacatgcaagctctgcccccagatgatga

SEQ ID NO: 56 - protein, 506 aa, Artificial Sequence
  Synthetic Construct
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSYVTVSSQDPAKDPKFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPDD

SEQ ID NO: 57 - protein, 268 aa, Artificial Sequence
  Synthetic Construct
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVS

SEQ ID NO: 58 - protein, 41 aa, Homo sapiens
KRGRKKLLYIFKQPFMRPVQTTQEEDGCSCRFPEEEEGGCE

SEQ ID NO: 59 - protein, 114 aa, Homo sapiens
LRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPDD

SEQ ID NO: 60 - DNA, 1953 bp, Artificial Sequence
  Synthetic Construct
atggagtttggactgagctggctgtttctggtcgccattctgaagggcgtgcagtgcggagaccaccctcaagctacacccgcccccgcccccgatgctagcaccgagctccccgccagcatgagccaagcccaacatctggccgctaacaccgccaccgacaactacagaatccccgccatcaccaccgctcccaatggagatctgctgatcagctatgacgagaggcccaaggataacggaaacggaggcagcgacgctcccaaccctaaccacatcgtccagagaaggtccacagatggcggaaagacatggtccgctcccacctacatccaccaaggcacagagaccggaaagaaggtcggctactccgaccccagctatgtcgtggatcaccagaccggcaccatcttcaacttccacgtgaagagctacgaccaaggctggggaggatccagaggcggcacagaccccgagaatagaggaattatccaagccgaggtctccaccagcaccgacaatggctggacatggacacatagaaccattaccgccgacattaccaaagacaagccttggacagccagattcgccgctagcggccaaggcatccagatccagcacggacctcacgctggcagactggtgcagcagtacaccattagaacagccggaggagctgtgcaagccgtctccgtgtattccgacgaccatggcaagacatggcaagccggcacccccattggcaccggcatggacgagaacaaggtggtggagctgtccgacggctctctgatgctcaactctagggcttccgatggcagcggattcagaaaggtggcccacagcaccgatggcggacagacatggagcgagcccgtgagcgacaagaatctgcccgactccgtggataacgcccagatcatcagagccttccctaacgctgcccccgacgatcctagagctaaagtgctgctgctgtcccacagccctaaccctagaccttggtccagagataggggcacaatcagcatgagctgcgacgatggcgccagctggacaaccagcaaagtgtttcacgagcccttcgtcggctacacaaccatcgctgtccaatccgacggatccatcggcctcctcagcgaagacgcccacaatggagctgactacggcggaatttggtatagaaacttcaccatgaattggctcggcgaacagtgcggacagaagcccgcctcctatgtgacagtcagctcccaagaccccgccaaggaccccaagttctgggtgctggtggtcgtgggaggagtgctggcttgctattctctgctcgtcaccgtggccttcatcatcttctgggtgaggtccaagaggagcagactgctgcacagcgactacatgaacatgacacctagaaggcccggccccacaaggaaacactaccaaccctacgccccccctagagatttcgccgcctataggagcaagaggggaaggaagaagctgctgtacattttcaagcagcccttcatgaggcccgtccaaaccacacaagaggaggacggatgtagctgtagattccccgaagaggaagagggaggatgcgaactgagagtgaaattctctaggagcgctgatgcccccgcctaccagcaaggccagaatcagctctacaacgagctcaatctgggcagaagagaggagtacgacgtgctggataagagaaggggaagggaccccgagatgggaggcaagcccagaagaaagaacccccaagagggactgtacaatgagctccagaaggacaagatggccgaagcctactccgaaatcggcatgaagggcgaaagaaggaggggcaaaggacacgacggactgtatcaaggcctctccaccgccaccaaagacacctacgatgctctgcacatgcaagctctccctcctagatgatga

SEQ ID NO: 61 - protein, 649 aa, Artificial Sequence
  Synthetic Construct
MEFGLSWLFLVAILKGVQCGDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPASYVTVSSQDPAKDPKFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 15 aa, Artificial Sequence
  Synthetic Construct
SYVTVSSQDPAKDPK

SEQ ID NO: 63 - protein, 68 aa, Homo sapiens
FWVLVVVGGVLACYSLLVTVAFIIFWVRSKRSRLLHSDYMNMTPRRPGPTRKHYQPYAPPRDFAAYRS

SEQ ID NO: 64 - DNA, 1305 bp, Artificial Sequence
  Synthetic Construct
atggagtttggactcagctggctgttcctcgtggccattctgaagggcgtccagtgcggcgatcaccctcaagctacccccgcccccgcccccgacgcctccacagagctccccgccagcatgtcccaagcccagcacctcgccgccaatacagctaccgacaactatagaatccccgctatcacaacagcccccaatggagatctgctgatctcctacgacgagagacccaaggataacggaaacggaggaagcgacgcccccaaccccaaccacatcgtgcagagaagaagcaccgacggcggaaagacatggtccgcccctacctacatccaccaaggcacagaaaccggcaagaaggtgggctacagcgacccctcctacgtggtggaccaccagaccggcaccatcttcaactttcacgtgaagtcctacgaccaaggctggggaggctccagaggcggaacagaccccgagaataggggcattatccaagccgaggtgtccacaagcacagacaacggatggacatggacccatagaaccatcacagccgacatcaccaaggataagccttggaccgctagatttgccgctagcggacaaggcatccagatccagcacggcccccacgctggaagactggtgcagcaatacaccatcagaaccgctggaggcgccgtgcaagctgtgagcgtctacagcgatgaccacggcaagacatggcaagccggaacccccattggcaccggcatggacgaaaacaaggtggtggagctgagcgacggatctctgatgctgaatagcagagcctccgatggcagcggattcagaaaggtggcccactccaccgatggcggacagacatggtccgaacccgtgtccgataagaatctgcccgactccgtggacaacgcccagatcattagagccttccctaatgccgctcccgacgaccccagagccaaggtgctgctgctgagccacagccctaaccctaggccttggagcagagatagaggcaccatcagcatgagctgcgatgacggcgctagctggaccacatccaaagtgttccacgagcctttcgtgggctataccaccatcgccgtgcagtccgacggctccattggactgctcagcgaggatgcccataatggcgccgactacggcggaatctggtatagaaacttcaccatgaactggctgggcgaacagtgtggccagaagcccgccaagaggaagaagaagggcggcaagaacggcaagaatagaaggaataggaaaaagaaaaatccttgatga

SEQ ID NO: 65 - protein, 433 aa, Artificial Sequence
  Synthetic Construct
MEFGLSWLFLVAILKGVQCGDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPAKRKKKGGKNGKNRRNRKKKNP

SEQ ID NO: 66 - protein, 24 aa, Homo sapiens
NGRRICLDLQAPLYKKIIKKLLES

SEQ ID NO: 67 - protein, 27 aa, Homo sapiens
GRELCLDPKENWVQRVVEKFLKRAENS

SEQ ID NO: 68 - protein, 34 aa, Homo sapiens
QIHFFFAKLNCRLYRKANKSSKLVSANRLFGDKS

SEQ ID NO: 69 - protein, 34 aa, Homo sapiens
ELRVRLASHLRKLRKRLLRDADDLQKRLAVYQAG

SEQ ID NO: 70 - protein, 12 aa, Homo sapiens
RRLRRMESESES

SEQ ID NO: 71 - protein, 21 aa, Homo sapiens
KRKKKGGKNGKNTTNTKKKNP